SEQUENCE LISTING (92 sequences)

SEQ ID NO: 1 - protein, 10 aa, Homo sapiens
GFTFSNYAMS

SEQ ID NO: 2 - protein, 17 aa, Homo sapiens
AINSNGGSAYYPDTVKD

SEQ ID NO: 3 - protein, 11 aa, Homo sapiens
RFYYDYGWFAY

SEQ ID NO: 4 - protein, 11 aa, Homo sapiens
RASENIDSYLA

SEQ ID NO: 5 - protein, 7 aa, Homo sapiens
AATLLAD

SEQ ID NO: 6 - protein, 9 aa, Homo sapiens
QHYYSSPWT

SEQ ID NO: 7 - protein, 120 aa, Homo sapiens
EVQLVESGGGLVKPGGSLKLSCAASGFTFSNYAMSWVRQTPEKRLEWVAAINSNGGSAYYPDTVKDRFTISRDNAKNTLYLQMSSLRSEDTALYYCARRFYYDYGWFAYWGQGTLVTVSS

SEQ ID NO: 8 - protein, 107 aa, Homo sapiens
DIVLTQSPASLSASVGETVTITCRASENIDSYLAWYQQKQGKSPQLLVYAATLLADGVPSRFSGSGSGTQYSLKINSLQSEDVARYYCQHYYSSPWTFGGGTKLEIK

SEQ ID NO: 9 - protein, 10 aa, Homo sapiens
GFSLSSYAMS

SEQ ID NO: 10 - protein, 16 aa, Homo sapiens
IISSSGTTYYASWAKG

SEQ ID NO: 11 - protein, 11 aa, Homo sapiens
VDYYSSGWGGL

SEQ ID NO: 12 - protein, 11 aa, Homo sapiens
QASEDIYSNLA

SEQ ID NO: 13 - protein, 7 aa, Homo sapiens
GASTLAS

SEQ ID NO: 14 - protein, 12 aa, Homo sapiens
QCTYDTSSYGNT

SEQ ID NO: 15 - protein, 116 aa, Homo sapiens
QSVEESGGRLVTPGTPLTLTCTVSGFSLSSYAMSWVRQAPGKGLEWIGIISSSGTTYYASWAKGRFTISKTSTTVDLKITSPTTEDTATYFCARVDYYSSGWGGLWGPGTLVTVSS

SEQ ID NO: 16 - protein, 110 aa, Homo sapiens
AVEMTQTPASMSAAVGGTVTIKCQASEDIYSNLAWYQQKPGQPPKLLIYGASTLASGVPSRFKGSGSGTEYTLTISGVQCDDAATYYCQCTYDTSSYGNTFGGGTEMVVE

SEQ ID NO: 17 - protein, 10 aa, Homo sapiens
GFSLSSYAMI

SEQ ID NO: 18 - protein, 16 aa, Homo sapiens
IINSSDNTHYATWAKG

SEQ ID NO: 19 - protein, 13 aa, Homo sapiens
DPYDYGYGWYFDL

SEQ ID NO: 20 - protein, 13 aa, Homo sapiens
QASQSVFNNKNLA

SEQ ID NO: 21 - protein, 7 aa, Homo sapiens
KASTLAS

SEQ ID NO: 22 - protein, 13 aa, Homo sapiens
QGEFSCSSTDCVT

SEQ ID NO: 23 - protein, 118 aa, Homo sapiens
QSVEESGGRLVTPGTPLTLTCTVSGFSLSSYAMIWVRQAPGKGLEWIGIINSSDNTHYATWAKGRFTISKTSTTVDLKITSPTTEDTATYFCARDPYDYGYGWYFDLWGPGTLVTVSS

SEQ ID NO: 24 - protein, 113 aa, Homo sapiens
AQVLTQTPSPVSAAVGGTVTINCQASQSVFNNKNLAWYQQKPGQPPKLLIYKASTLASGVSSRFKGSGSGTQFALTISGVQCDDAATYYCQGEFSCSSTDCVTFGGGTEVVVK

SEQ ID NO: 25 - protein, 10 aa, Homo sapiens
GFSLSTYAMS

SEQ ID NO: 26 - protein, 16 aa, Homo sapiens
IISSSGSTYYASWAKG

SEQ ID NO: 27 - protein, 8 aa, Homo sapiens
ETWYWLDL

SEQ ID NO: 28 - protein, 11 aa, Homo sapiens
QASEDIYSNLA

SEQ ID NO: 29 - protein, 7 aa, Homo sapiens
GASTLAS

SEQ ID NO: 30 - protein, 12 aa, Homo sapiens
QCAYDSSSYGTP

SEQ ID NO: 31 - protein, 113 aa, Homo sapiens
QSVEESGGRLVTPGTPLTLTCTASGFSLSTYAMSWVRQAPGKGLEWIGIISSSGSTYYASWAKGRFTISKTSTTVDLKITSPTTEDTATYFCARETWYWLDLWGQGTLVTVSS

SEQ ID NO: 32 - protein, 110 aa, Homo sapiens
AVEMTQTPASVSAAVGGTVTINCQASEDIYSNLAWYQQKPGQPPKLLIYGASTLASGVPSRFKGSGSGTEYTLTISGVQCDDAATYYCQCAYDSSSYGTPFGGGTEVVVK

SEQ ID NO: 33 - protein, 10 aa, Homo sapiens
GFSLSNYAMS

SEQ ID NO: 34 - protein, 16 aa, Homo sapiens
IIYTTGFTYYASWVKG

SEQ ID NO: 35 - protein, 14 aa, Homo sapiens
GLATYVSPPTRLDL

SEQ ID NO: 36 - protein, 13 aa, Homo sapiens
QASQSIYNSKNLA

SEQ ID NO: 37 - protein, 7 aa, Homo sapiens
SASTLAS

SEQ ID NO: 38 - protein, 13 aa, Homo sapiens
QGEFSCSSVDCAT

SEQ ID NO: 39 - protein, 119 aa, Homo sapiens
QSVEESGGRLVTPGTPLTLTCTVSGFSLSNYAMSWVRQAPGKGLEWIGIIYTTGFTYYASWVKGRFTISKTSTTVDLKITSPTTEDTATYFCARGLATYVSPPTRLDLWGQGTLVTVSS

SEQ ID NO: 40 - protein, 113 aa, Homo sapiens
AQVLTQTPSPVSAAVGGTVTINCQASQSIYNSKNLAWYQQKPGQPPKLLIYSASTLASGVPSRFKGSGSGTQFTLTISDLECDDAATYYCQGEFSCSSVDCATFGGGTEVVVK

SEQ ID NO: 41 - protein, 444 aa, Homo sapiens
EVQLVESGGGLVKPGGSLKLSCAASGFTFSNYAMSWVRQTPEKRLEWVAAINSNGGSAYYPDTVKDRFTISRDNAKNTLYLQMSSLRSEDTALYYCARRFYYDYGWFAYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 42 - protein, 214 aa, Homo sapiens
DIVLTQSPASLSASVGETVTITCRASENIDSYLAWYQQKQGKSPQLLVYAATLLADGVPSRFSGSGSGTQYSLKINSLQSEDVARYYCQHYYSSPWTFGGGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 43 - protein, 440 aa, Homo sapiens
QSVEESGGRLVTPGTPLTLTCTVSGFSLSSYAMSWVRQAPGKGLEWIGIISSSGTTYYASWAKGRFTISKTSTTVDLKITSPTTEDTATYFCARVDYYSSGWGGLWGPGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 44 - protein, 217 aa, Homo sapiens
AVEMTQTPASMSAAVGGTVTIKCQASEDIYSNLAWYQQKPGQPPKLLIYGASTLASGVPSRFKGSGSGTEYTLTISGVQCDDAATYYCQCTYDTSSYGNTFGGGTEMVVERTDAAPTVSIFPPSSEQLTSGGASVVCFLNNFYPKDINVKWKIDGSERQNGVLNSWTDQDSKDCTYSMSSTLTLTKDEYERHNSYTCEATHKTSTSPIVKSFNRNEC

SEQ ID NO: 45 - protein, 442 aa, Homo sapiens
QSVEESGGRLVTPGTPLTLTCTVSGFSLSSYAMIWVRQAPGKGLEWIGIINSSDNTHYATWAKGRFTISKTSTTVDLKITSPTTEDTATYFCARDPYDYGYGWYFDLWGPGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 46 - protein, 220 aa, Homo sapiens
AQVLTQTPSPVSAAVGGTVTINCQASQSVFNNKNLAWYQQKPGQPPKLLIYKASTLASGVSSRFKGSGSGTQFALTISGVQCDDAATYYCQGEFSCSSTDCVTFGGGTEVVVKRTDAAPTVSIFPPSSEQLTSGGASVVCFLNNFYPKDINVKWKIDGSERQNGVLNSWTDQDSKDCTYSMSSTLTLTKDEYERHNSYTCEATHKTSTSPIVKSFNRNEC

SEQ ID NO: 47 - protein, 437 aa, Homo sapiens
QSVEESGGRLVTPGTPLTLTCTASGFSLSTYAMSWVRQAPGKGLEWIGIISSSGSTYYASWAKGRFTISKTSTTVDLKITSPTTEDTATYFCARETWYWLDLWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 48 - protein, 217 aa, Homo sapiens
AVEMTQTPASVSAAVGGTVTINCQASEDIYSNLAWYQQKPGQPPKLLIYGASTLASGVPSRFKGSGSGTEYTLTISGVQCDDAATYYCQCAYDSSSYGTPFGGGTEVVVKRTDAAPTVSIFPPSSEQLTSGGASVVCFLNNFYPKDINVKWKIDGSERQNGVLNSWTDQDSKDCTYSMSSTLTLTKDEYERHNSYTCEATHKTSTSPIVKSFNRNEC

SEQ ID NO: 49 - protein, 443 aa, Homo sapiens
QSVEESGGRLVTPGTPLTLTCTVSGFSLSNYAMSWVRQAPGKGLEWIGIIYTTGFTYYASWVKGRFTISKTSTTVDLKITSPTTEDTATYFCARGLATYVSPPTRLDLWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 50 - protein, 220 aa, Homo sapiens
AQVLTQTPSPVSAAVGGTVTINCQASQSIYNSKNLAWYQQKPGQPPKLLIYSASTLASGVPSRFKGSGSGTQFTLTISDLECDDAATYYCQGEFSCSSVDCATFGGGTEVVVKRTDAAPTVSIFPPSSEQLTSGGASVVCFLNNFYPKDINVKWKIDGSERQNGVLNSWTDQDSKDCTYSMSSTLTLTKDEYERHNSYTCEATHKTSTSPIVKSFNRNEC

SEQ ID NO: 51 - DNA, 429 bp, Homo sapiens
aagcttgccaccatggaatggagctgggtctttctcttcttcctgtcagtaactacaggagtccattctgaggtgcagctggtggagtctgggggaggcttagtgaagcctggagggtccctgaaactctcctgtgcagcctctggattcactttcagtaactatgccatgtcttgggttcgccagactccagagaagaggctggagtgggtcgcagccattaatagtaatggtggtagcgcctactatccagacactgtgaaggaccgattcaccatctccagagacaatgccaagaacaccctgtacctgcaaatgagcagtctgaggtctgaggacacagccttgtattactgtgcaagacgcttctactatgattacggctggtttgcttactggggccaagggactctggtcacagtctcgagc

SEQ ID NO: 52 - DNA, 399 bp, Homo sapiens
aagcttgccaccatgtctgtccccacccaagtcctcggactcctgctactctggcttacagatgccagatgcgacattgtgctgacccaatctccagcttccctgtctgcatctgtgggagaaactgtcaccatcacatgtcgagcaagtgagaatattgacagttatttagcatggtatcagcagaaacagggaaaatctcctcagctcctggtctatgctgcaacactcttagcagatggtgtgccatcaaggttcagtggcagtggatcaggcacacagtattctctcaagatcaacagcctgcagtctgaagatgttgcgagatattactgtcaacattattatagttctccgtggacgttcggtggaggcaccaagctggaaataaaacgtacg

SEQ ID NO: 53 - DNA, 417 bp, Homo sapiens
aagcttgccaccatggaatggagctgggtctttctcttcttcctgtcagtaactacaggagtccattctcagtcggtggaggagtccgggggtcgcctggtcacgcctgggacacccctgacactcacatgcacagtctctggattctccctcagtagctatgcgatgagctgggtccgccaggctccagggaaggggctggaatggatcggaatcattagtagcagtggtaccacatactacgcgagctgggcgaaaggccgattcaccatttccaaaacctcgaccacggtggatctgaaaatcaccagtccgacaaccgaggacacggccacctatttctgtgccagagtcgattactatagtagtggctggggtggcttgtggggcccaggcaccctggtcaccgtctcgagc

SEQ ID NO: 54 - DNA, 418 bp, Homo sapiens
aagcttcgaagccaccatggacacgagggcccccactcagctgctggggctcctgctgctctggctcccaggtgccacatttgccgttgaaatgacccagactccagcctcgatgtctgccgctgtgggaggcacagtcaccatcaagtgccaggccagtgaggacatttatagcaatttggcctggtatcagcagaaaccagggcagcctcccaagctcctgatctatggtgcatccactctggcttctggggtcccatcgcggttcaaaggcagtggatctgggacagagtacactctcaccatcagcggtgtgcagtgtgacgatgctgccacttactattgtcaatgcacttatgatactagtagttatggtaatactttcggcggagggaccgagatggtagtcgaacgtacg

SEQ ID NO: 55 - DNA, 423 bp, Homo sapiens
aagcttgccaccatggaatggagctgggtctttctcttcttcctgtcagtaactacaggagtccattctcagtcggtggaggagtccgggggtcgcctggtcacgcctgggacacccctgacactcacctgcacagtctctggattctccctcagtagctatgcaatgatctgggtccgccaggctccagggaaggggctggaatggatcggaatcattaatagtagtgataacacacactacgcgacctgggcgaaaggccgattcaccatctccaaaacctcgaccacggtggatctaaaaatcaccagtccgacaaccgaggacacggccacctatttctgtgccagagatccctacgactatggttatggttggtactttgacttgtggggcccaggcaccctggtcaccgtctcgagc

SEQ ID NO: 56 - DNA, 427 bp, Homo sapiens
aagcttcgaagccaccatggacacgagggcccccactcagctgctggggctcctgctgctctggctcccaggtgccacatttgcccaagtgctgacccagactccatcccctgtgtctgcagctgtgggaggcacagtcaccatcaactgccaggccagtcagagtgtttttaataacaaaaatttagcctggtatcagcagaaaccagggcagcctcccaagctcctgatctacaaggcatccactctggcatctggcgtctcatcgcggttcaaaggcagtggatctgggacacagttcgctctcaccatcagcggcgtgcagtgtgacgatgctgccacttactactgtcaaggcgaatttagttgtagtagtactgattgcgtgactttcggcggagggaccgaggtggtggtcaaacgtacg

SEQ ID NO: 57 - DNA, 408 bp, Homo sapiens
aagcttgccaccatggaatggagctgggtctttctcttcttcctgtcagtaactacaggagtccattctcagtcggtggaggagtccgggggtcgcctggtcacgcctgggacacccctgacactcacctgcacagcctctggattctccctcagtacctatgcaatgagttgggtccgccaggctccagggaaggggctggaatggatcggaatcattagtagtagtggtagcacatactacgcgagctgggcgaaaggccgattcaccatctccaaaacctcgaccacggtggatctgaaaatcaccagtccgacaaccgaggacacggccacctatttctgtgccagagagacttggtactggttggatctctggggccagggcaccctggtcaccgtctcgagc

SEQ ID NO: 58 - DNA, 418 bp, Homo sapiens
aagcttcgaagccaccatggacatgagggcccccactcagctgctggggctcctgctgctctggctcccaggtgccacatttgccgttgaaatgacccagactccagcctcggtgtctgccgctgtgggaggcacagtcaccatcaattgccaggccagtgaggacatttatagcaatttggcctggtatcagcagaaaccagggcagcctcccaagctcctgatctatggtgcatccactctggcatctggggtcccatcgcggttcaaaggcagtggatctgggacagagtacactctcaccatcagcggtgtgcagtgtgacgatgctgccacttactactgtcaatgcgcttatgatagtagtagttatggtacccctttcggcggagggaccgaggtggtggtcaaacgtacg

SEQ ID NO: 59 - DNA, 426 bp, Homo sapiens
aagcttgccaccatggaatggagctgggtctttctcttcttcctgtcagtaactacaggagtccattctcagtcggtggaggagtccgggggtcgcctggtcacgcctgggacacccctgacactcacctgcacagtctctggattctccctcagtaactatgcaatgagctgggtccgccaggctccagggaaggggctggaatggatcggaatcatttatactactggtttcacatactacgcgagctgggtgaaaggccgattcaccatctccaaaacctcgaccacggtggacctgaaaatcaccagtccgacaaccgaggacacggccacctatttctgtgccagagggctggctacttatgttagtcccccgactcggttggatctctggggccagggcaccctggtcaccgtctcgagc

SEQ ID NO: 60 - DNA, 427 bp, Homo sapiens
aagcttcgaagccaccatgaacatgagggcccccactcagctgctggggctcctgctgctctggctcccaggtgccacatttgcccaagtgctgacccagactccatcccctgtgtctgcagctgtgggaggcacagtcaccatcaactgccaggccagtcagagtatttataatagcaaaaatttagcctggtatcagcagaaaccagggcagcctcccaagctcctgatctattctgcatccactctggcatctggggtcccatcgcggttcaaaggcagtggatctgggacacagttcactctcaccatcagcgacctggagtgtgacgatgctgccacttactactgtcaaggcgaatttagttgtagtagtgttgattgcgccactttcggcggagggaccgaggtggtggtcaaacgtacg

SEQ ID NO: 61 - DNA, 30 bp, Homo sapiens
ggattcactttcagtaactatgccatgtct

SEQ ID NO: 62 - DNA, 51 bp, Homo sapiens
gccattaatagtaatggtggtagcgcctactatccagacactgtgaaggac

SEQ ID NO: 63 - DNA, 33 bp, Homo sapiens
cgcttctactatgattacggctggtttgcttac

SEQ ID NO: 64 - DNA, 33 bp, Homo sapiens
cgagcaagtgagaatattgacagttatttagca

SEQ ID NO: 65 - DNA, 21 bp, Homo sapiens
gctgcaacactcttagcagat

SEQ ID NO: 66 - DNA, 27 bp, Homo sapiens
caacattattatagttctccgtggacg

SEQ ID NO: 67 - DNA, 30 bp, Homo sapiens
ggattctccctcagtagctatgcgatgagc

SEQ ID NO: 68 - DNA, 48 bp, Homo sapiens
atcattagtagcagtggtaccacatactacgcgagctgggcgaaaggc

SEQ ID NO: 69 - DNA, 33 bp, Homo sapiens
gtcgattactatagtagtggctggggtggcttg

SEQ ID NO: 70 - DNA, 33 bp, Homo sapiens
caggccagtgaggacatttatagcaatttggcc

SEQ ID NO: 71 - DNA, 21 bp, Homo sapiens
ggtgcatccactctggcttct

SEQ ID NO: 72 - DNA, 36 bp, Homo sapiens
caatgcacttatgatactagtagttatggtaatact

SEQ ID NO: 73 - DNA, 30 bp, Homo sapiens
ggattctccctcagtagctatgcaatgatc

SEQ ID NO: 74 - DNA, 48 bp, Homo sapiens
atcattaatagtagtgataacacacactacgcgacctgggcgaaaggc

SEQ ID NO: 75 - DNA, 39 bp, Homo sapiens
gatccctacgactatggttatggttggtactttgacttg

SEQ ID NO: 76 - DNA, 39 bp, Homo sapiens
caggccagtcagagtgtttttaataacaaaaatttagcc

SEQ ID NO: 77 - DNA, 21 bp, Homo sapiens
aaggcatccactctggcatct

SEQ ID NO: 78 - DNA, 39 bp, Homo sapiens
caaggcgaatttagttgtagtagtactgattgcgtgact

SEQ ID NO: 79 - DNA, 30 bp, Homo sapiens
ggattctccctcagtacctatgcaatgagt

SEQ ID NO: 80 - DNA, 48 bp, Homo sapiens
atcattagtagtagtggtagcacatactacgcgagctgggcgaaaggc

SEQ ID NO: 81 - DNA, 24 bp, Homo sapiens
gagacttggtactggttggatctc

SEQ ID NO: 82 - DNA, 33 bp, Homo sapiens; identical to SEQ ID NO: 70

SEQ ID NO: 83 - DNA, 21 bp, Homo sapiens
ggtgcatccactctggcatct

SEQ ID NO: 84 - DNA, 36 bp, Homo sapiens
caatgcgcttatgatagtagtagttatggtacccct

SEQ ID NO: 85 - DNA, 30 bp, Homo sapiens
ggattctccctcagtaactatgcaatgagc

SEQ ID NO: 86 - DNA, 48 bp, Homo sapiens
atcatttatactactggtttcacatactacgcgagctgggtgaaaggc

SEQ ID NO: 87 - DNA, 42 bp, Homo sapiens
gggctggctacttatgttagtcccccgactcggttggatctc

SEQ ID NO: 88 - DNA, 39 bp, Homo sapiens
caggccagtcagagtatttataatagcaaaaatttagcc

SEQ ID NO: 89 - DNA, 21 bp, Homo sapiens
tctgcatccactctggcatct

SEQ ID NO: 90 - DNA, 39 bp, Homo sapiens
caaggcgaatttagttgtagtagtgttgattgcgccact

SEQ ID NO: 91 - DNA, 32 bp, Artificial Sequence
  Made in Lab - primer
atggtaccaagaggaatgtaaatgtgtccggc

SEQ ID NO: 92 - DNA, 34 bp, Artificial Sequence
  Made in lab - primer
aagcggccgcgatcatgttaaccaaggtcaggaa

(19) United States
(12) Patent Application Publication
(10) Pub. No.: US 2024/0383986 A1
(43) Pub. Date: Nov. 21, 2024

(54) ANTIBODIES

(71) Applicant: Oxford University Innovation Limited, Oxford (GB)

(72) Inventors: Graham OGG, Oxford (Oxfordshire) (GB); Clare HARDMAN, Oxford (Oxfordshire) (GB); Yi-Ling CHEN, Oxford (Oxfordshire) (GB)

(21) Appl. No.: 18/563,440

(22) PCT Filed: May 20, 2022
(86) PCT No.: PCT/GB2022/051285

§ 371 Date: Nov. 22, 2023

(30) Foreign Application Priority Data
May 26, 2021 (GB) 2107517.1
Nov. 19, 2021 (GB) 2116709.3

(51) Int. Cl. C07K 16/28; A61P 29/00; A61P 37/06; G01N 33/53; G01N 33/68

(52) U.S. Cl. CPC C07K 16/2833; A61P 29/00; A61P 37/06; G01N 33/53; G01N 33/6893; C07K 2317/24; C07K 2317/31; C07K 2317/565; C07K 2317/622; C07K 2317/76; G01N 2800/24; G01N 2800/52

(57) ABSTRACT

The invention relates to an antibody or antigen binding fragment thereof which is capable of binding to CD1a, which is particularly suitable for treating or preventing one or more inflammatory skin or mucosal disorder, or disease or one or more associated systemic disease or disorder, or one or more inflammatory drug reaction which manifests systemically, or a CD1a-expressing malignancy

### FIELD OF INVENTION

The present invention relates to antibodies, and their use in treating, preventing or monitoring inflammatory skin and mucosal diseases or disorders, or associated systemic diseases or disorders, or inflammatory drug reactions which manifest systemically, or CD1a-expressing malignancies.

### BACKGROUND

Antigen presentation is one of the fundamental pillars of host immunity, by which the immune system detects threats including infection, tissue damage and disease, and orchestrates a tailored defence. Antigen presentation encompasses antigen internalisation, processing and display by presentation molecules on the surface of specialised antigen-presenting cells (APCs). Presentation of antigen is organised to achieve optimal activation of the immune response targeted to the antigen source and eliminate the threat. Antigens encompass a broad range of molecules including peptides, lipids and metabolites and others. MHCI and MHCII are proteins expressed on the surface of APCs which bind to peptide antigens and largely present to CD8+ T cells and CD4+ T cells respectively. These T cell subsets are induced to exert their effector functions upon recognition of the MHC-bound peptide antigen by the cell surface T-cell receptor (TCR) enabling immunity to pathogens and to cancers. However, dysregulated presentation of innocuous antigens, such as allergens in allergic diseases, or self-proteins in autoimmunity causes host damage, inflammation and disease. Therefore, targeting of the antigen presentation pathway is a powerful means of modulating the ensuing immune response.

CD1 molecules constitute a family of antigen presentation molecules structurally akin to MHCI. In contrast, CD1 molecules are relatively non-polymorphic and the CD1 antigen binding groove is enriched in hydrophobic amino acids enabling presentation of lipid species. Lipids are important antigens forming vital components of host and pathogen cell membranes and are less subject to mutation than protein-derived peptide antigens. The CD1 family is made up of cell surface group-1 molecules CD1a/b/c and group-2 CD1d and group-3 CD1e. Most of the understanding of CD1 lipid presentation and T cell responses has come from study of invariant Natural Killer T cell recognition of glycolipid bound CD1d, partly because CD1d is the only CD1 normally expressed by mice. CD1d and MHCI molecules are broadly expressed whereas MHCII and group 1 CD1 expression is relatively restricted to APCs. However, CD1a unique among these molecules is highly specific to the skin and mucosae. CD1a is constitutively expressed by Langerhans cells (LCs) in the epidermis of skin and mucosae (1) and is commonly used as an identifying marker for LCs, in addition to langerin. Additionally, CD1a is expressed at lower levels on subsets of dermal dendritic cells (2-4) and can be expressed and upregulated on skin innate lymphoid cells (ILCs), in particular ILC2 (5). Importantly, CD1a was first described on the surface of immature thymocytes, but expression is typically lost upon T cell maturation (6). The high level of constitutive expression of CD1a in the skin is indicative of an important physiological role for CD1a-dependent surveillance and T cell activation in healthy and diseased human skin. Moreover, the increase in CD1a expression in atopic dermatitis skin may underlie the increased activation of CD1a-reactive T cell populations in inflammatory skin disease.

T cell responses directed by CD1a, CD1b, or CD1c molecules presenting mycobacterial lipid-based antigens have been implicated in human immune responses to Mycobacterium tuberculosis and Mycobacterium leprae infections. Recognition of other, more common pathogenic or commensal bacterial lipids by CD1a-restricted T cells is the subject of ongoing studies, with some data presented herein. Whereas TCR recognition of peptide antigens by MHC-restricted T cells is generally highly specific for the peptide antigen, the CD1 mode of TCR recognition is more diverse with highly lipid-specific responses (7) and cross-reactive or even apparently lipid independent signalling mediated by direct TCR-CD1 interaction (8-10), as is the case for CD1a-autoreactive T cells. CD1a-autoreactive T cells are activated in some cases upon recognition of small hydrophobic host-derived lipids that nest within the CD1a antigen binding groove and do not protrude, allowing the TCR to interact with the CD1a protein itself, rather than with the lipid. In this case binding of lipids with large or charged headgroups would prevent the interaction between an autoreactive TCR and CD1a, thereby preventing T cell activation (11, 12).

CD1a is relatively non-polymorphic, and so there is therefore population-wide potential in prevention and/or treatment of inflammatory skin and mucosal diseases and disorders, such as atopic dermatitis, psoriasis, lupus erythematosus, or associated systemic diseases or disorders, or inflammatory drug reactions which manifest systemically, where the frequency of CD1a-expressing dendritic cell subsets is altered, and migratory patterns of LCs or responding T cells are altered (13-15). Furthermore, CD1a has been linked to other systemic disorders including inflammatory bowel disease, multiple sclerosis, Guillain-Barre syndrome, thyroiditis, and neurodegeneration (Al-amodi Inflammatory Bowel Diseases 2018 24: 1225-1236; Caporale J Neuroimmunol 2006 177:112-8; Jamshidian Immunological Investigations 2010 3:874-889; Roura-Mir J Immunol 2005 174:3773-80; Wang Aging 2019 11: 4521-4535). In addition, CD1a can be expressed by certain malignancies including Langerhans cell histiocytosis, subsets of T cell lymphomas, subsets of thymomas and rare descriptions of other malignancies, such as subsets of mastocytosis.

It is an object of the invention to provide anti-CD1a antibodies. Such antibodies are particularly useful in treating or preventing inflammatory diseases or disorders of the skin or mucosa, such as psoriasis, dermatitis, lupus erythematosus or drug reactions which manifest as an inflammatory skin or mucosal disease or disorder. Such antibodies may also be beneficial in treating or preventing associated systemic diseases or disorders, or inflammatory drug reactions which manifest systemically or in the treatment of CD1a-expressing malignancies.

### SUMMARY OF INVENTION

In an aspect, the invention provides an antibody or antigen binding fragment thereof which is capable of binding to CD1a. The antibody or antigen binding fragment thereof may specifically bind to CD1a. The antibody or antigen binding fragment thereof may preferentially bind to CD1a. The antibody or antigen binding fragment thereof may induce cell death of cells expressing CD1a. The antibody or antigen binding fragment thereof may block the binding of ligands to CD1a.

The antibody or antigen binding fragment thereof may comprise a heavy chain variable region comprising a complementarity determining region (CDR) 3 (CDR3) of SEQ ID NO: 3 or a sequence having at least 80%, 90%, 95%, 98%, 99% or 100% identity thereto; and/or
the antibody or antigen binding fragment thereof may comprise a light chain variable region comprising a CDR3 of SEQ ID NO:6 or a sequence having at least 80%, 90%, 95%, 98%, 99% or 100% identity thereto.

The antibody or antigen binding fragment thereof may comprise a heavy chain variable region comprising a CDR3 of SEQ ID NO: 11 or a sequence having at least 80%, 90%, 95%, 98%, 99% or 100% identity thereto; and/or
the antibody or antigen binding fragment thereof may comprise a light chain variable region comprising a CDR3 of SEQ ID NO: 14 or a sequence having at least 80%, 90%, 95%, 98%, 99% or 100% identity thereto.

The antibody or antigen binding fragment thereof may comprise a heavy chain variable region comprising a CDR3 of SEQ ID NO: 19 or a sequence having at least 80%, 90%, 95%, 98%, 99% or 100% identity thereto; and/or
the antibody or antigen binding fragment thereof may comprise a light chain variable region comprising a CDR3 of SEQ ID NO: 22 or a sequence having at least 80%, 90%, 95%, 98%, 99% or 100% identity thereto.

The antibody or antigen binding fragment thereof may comprise a heavy chain variable region comprising a CDR3 of SEQ ID NO: 27 or a sequence having at least 80%, 90%, 95%, 98%, 99% or 100% identity thereto; and/or
the antibody or antigen binding fragment thereof may comprise a light chain variable region comprising a CDR3 of SEQ ID NO: 30 or a sequence having at least 80%, 90%, 95%, 98%, 99% or 100% identity thereto.

The antibody or antigen binding fragment thereof may comprise a heavy chain variable region comprising a CDR3 of SEQ ID NO: 35 or a sequence having at least 80%, 90%, 95%, 98%, 99% or 100% identity thereto; and/or

The antibody or antigen binding fragment thereof may comprise a light chain variable region comprising a CDR3 of SEQ ID NO: 38 or a sequence having at least 80%, 90%, 95%, 98%, 99% or 100% identity thereto.

The antibody or antigen binding fragment thereof may comprise or consist of:
a) a heavy chain variable region comprising:
   a CDR1 of SEQ ID NO: 1,
   a CDR2 of SEQ ID NO: 2, and
   a CDR3 of SEQ ID NO: 3,
      or sequences having at least 80%, 90%, 95%, 98%, 99% or 100% identity thereto; and/or
b) a light chain variable region comprising:
   a CDR1 of SEQ ID NO: 4,
   a CDR2 of SEQ ID NO: 5, and
   a CDR3 of SEQ ID NO: 6,
      or sequences having at least 80%, 90%, 95%, 98%, 99% or 100% identity thereto.

The antibody or antigen binding fragment thereof may comprise or consist of:
a) a heavy chain variable region comprising:
   a CDR1 of SEQ ID NO: 9,
   a CDR2 of SEQ ID NO: 10, and
   a CDR3 of SEQ ID NO: 11,
      or sequences having at least 80%, 90%, 95%, 98%, 99% or 100% identity thereto; and/or
b) a light chain variable region comprising:
   a CDR1 of SEQ ID NO: 12,
   a CDR2 of SEQ ID NO: 13, and
   a CDR3 of SEQ ID NO: 14,
      or sequences having at least 80%, 90%, 95%, 98%, 99% or 100% identity thereto.

The antibody or antigen binding fragment thereof may comprise or consist of:
a) a heavy chain variable region comprising:
   a CDR1 of SEQ ID NO: 17,
   a CDR2 of SEQ ID NO: 18, and
   a CDR3 of SEQ ID NO: 19,
      or sequences having at least 80%, 90%, 95%, 98%, 99% or 100% identity thereto; and/or
b) a light chain variable region comprising:
   a CDR1 of SEQ ID NO: 20,
   a CDR2 of SEQ ID NO: 21, and
   a CDR3 of SEQ ID NO: 22,
      or sequences having at least 80%, 90%, 95%, 98%, 99% or 100% identity thereto.

The antibody or antigen binding fragment thereof may comprise or consist of:
a) a heavy chain variable region comprising:
   a CDR1 of SEQ ID NO: 25,
   a CDR2 of SEQ ID NO: 26, and
   a CDR3 of SEQ ID NO: 27,
      or sequences having at least 80%, 90%, 95%, 98%, 99% or 100% identity thereto; and/or
b) a light chain variable region comprising:
   a CDR1 of SEQ ID NO: 28,
   a CDR2 of SEQ ID NO: 29, and
   a CDR3 of SEQ ID NO: 30,
      or sequences having at least 80%, 90%, 95%, 98%, 99% or 100% identity thereto.

The antibody or antigen binding fragment thereof may comprise or consist of:
a) a heavy chain variable region comprising:
   a CDR1 of SEQ ID NO: 33,
   a CDR2 of SEQ ID NO: 34, and
   a CDR3 of SEQ ID NO: 35,
      or sequences having at least 80%, 90%, 95%, 98%, 99% or 100% identity thereto; and/or
b) a light chain variable region comprising:
   a CDR1 of SEQ ID NO: 36,
   a CDR2 of SEQ ID NO: 37, and
   a CDR3 of SEQ ID NO: 38
      or sequences having at least 80%, 90%, 95%, 98%, 99% or 100% identity thereto.

The CDRs may be associated with any framework region. Preferably, the framework region is of human origin.

The antibody or antigen binding fragment thereof may comprise or consist of:
a) a heavy chain variable region comprising or consisting of SEQ ID NO: 7 or a sequence having at least 80%, 90%, 95%, 98%, 99% or 100% identity thereto; and/or
b) a light chain variable region comprising or consisting of SEQ ID NO: 8.

or a sequence having at least 80%, 90%, 95%, 98%, 99% or 100% identity thereto.

The antibody or antigen binding fragment thereof may comprise or consist of:
a) a heavy chain variable region comprising or consisting of SEQ ID NO: 15 or a sequence having at least 80%, 90%, 95%, 98%, 99% or 100% identity thereto: and/or
b) a light chain variable region comprising or consisting of SEQ ID NO: 16
   or a sequence having at least 80%, 90%, 95%, 98%, 99% or 100% identity thereto.

The antibody or antigen binding fragment thereof may comprise or consist of:
a) a heavy chain variable region comprising or consisting of SEQ ID NO: 23 or a sequence having at least 80%, 90%, 95%, 98%, 99% or 100% identity thereto; and/or
b) a light chain variable region comprising or consisting of SEQ ID NO: 24
   or a sequence having at least 80%, 90%, 95%, 98%, 99% or 100% identity thereto.

The antibody or antigen binding fragment thereof may comprise or consist of:
a) a heavy chain variable region comprising or consisting of SEQ ID NO: 31 or a sequence having at least 80%, 90%, 95%, 98%, 99% or 100% identity thereto; and/or
b) a light chain variable region comprising or consisting of SEQ ID NO: 32
   or a sequence having at least 80%, 90%, 95%, 98%, 99% or 100% identity thereto.

The antibody or antigen binding fragment thereof may comprise or consist of:
a) a heavy chain variable region comprising or consisting of SEQ ID NO: 39 or a sequence having at least 80%, 90%, 95%, 98%, 99% or 100% identity thereto; and/or
b) a light chain variable region comprising or consisting of SEQ ID NO: 40
   or a sequence having at least 80%, 90%, 95%, 98%, 99% or 100% identity thereto.

The antibody or antigen binding fragment thereof may consist of:
a) a heavy chain variable region comprising or consisting of SEQ ID NO: 7; and
b) a light chain variable region comprising or consisting of SEQ ID NO: 8.

The antibody or antigen binding fragment thereof may consist of:
a) a heavy chain variable region comprising or consisting of SEQ ID NO: 15; and
b) a light chain variable region comprising or consisting of SEQ ID NO: 16.

The antibody or antigen binding fragment thereof may consist of:
a) a heavy chain variable region comprising or consisting of SEQ ID NO: 23; and
b) a light chain variable region comprising or consisting of SEQ ID NO: 24.

The antibody or antigen binding fragment thereof may consist of:
a) a heavy chain variable region comprising or consisting of SEQ ID NO: 31; and
b) a light chain variable region comprising or consisting of SEQ ID NO: 32.

The antibody or antigen binding fragment thereof may consist of:
a) a heavy chain variable region comprising or consisting of SEQ ID NO: 39; and
b) a light chain variable region comprising or consisting of SEQ ID NO: 40.

The antibody or antigen binding fragment thereof may comprise or consist of:
a) a heavy chain comprising or consisting of SEQ ID NO: 41 or a sequence having at least 80%, 90%, 95%, 98%, 99% or 100% identity thereto; and/or
b) a light chain comprising or consisting of SEQ ID NO: 42
   or a sequence having at least 80%, 90%, 95%, 98%, 99% or 100% identity thereto.

The antibody or antigen binding fragment thereof may comprise or consist of:
a) a heavy chain comprising or consisting of SEQ ID NO: 43 or a sequence having at least 80%, 90%, 95%, 98%, 99% or 100% identity thereto; and/or
b) a light chain comprising or consisting of SEQ ID NO: 44
   or a sequence having at least 80%, 90%, 95%, 98%, 99% or 100% identity thereto.

The antibody or antigen binding fragment thereof may comprise or consist of:
a) a heavy chain comprising or consisting of SEQ ID NO: 45 or a sequence having at least 80%, 90%, 95%, 98%, 99% or 100% identity thereto; and/or
b) a light chain comprising or consisting of SEQ ID NO: 46
   or a sequence having at least 80%, 90%, 95%, 98%, 99% or 100% identity thereto.

The antibody or antigen binding fragment thereof may comprise or consist of:
a) a heavy chain comprising or consisting of SEQ ID NO: 47 or a sequence having at least 80%, 90%, 95%, 98%, 99% or 100% identity thereto; and/or
b) a light chain comprising or consisting of SEQ ID NO: 48
   or a sequence having at least 80%, 90%, 95%, 98%, 99% or 100% identity thereto.

The antibody or antigen binding fragment thereof may comprise or consist of:
a) a heavy chain comprising or consisting of SEQ ID NO: 49 or a sequence having at least 80%, 90%, 95%, 98%, 99% or 100% identity thereto; and/or
b) a light chain comprising or consisting of SEQ ID NO: 50
   or a sequence having at least 80%, 90%, 95%, 98%, 99% or 100% identity thereto.

The antibody or antigen binding fragment thereof may consist of:
a) a heavy chain comprising or consisting of SEQ ID NO: 41; and
b) a light chain comprising or consisting of SEQ ID NO: 42.

The antibody or antigen binding fragment thereof may consist of:
a) a heavy chain comprising or consisting of SEQ ID NO: 43; and
b) a light chain comprising or consisting of SEQ ID NO: 44.

The antibody or antigen binding fragment thereof may consist of:
a) a heavy chain comprising or consisting of SEQ ID NO: 45; and
b) a light chain comprising or consisting of SEQ ID NO: 46.

The antibody or antigen binding fragment thereof may consist of:
a) a heavy chain comprising or consisting of SEQ ID NO: 47; and
b) a light chain comprising or consisting of SEQ ID NO: 48.

The antibody or antigen binding fragment thereof may consist of:
a) a heavy chain comprising or consisting of SEQ ID NO: 49; and
b) a light chain comprising or consisting of SEQ ID NO: 50.

An antibody or antigen binding fragment thereof of the invention may be isolated.

In any aspect, “an antibody or antigen binding fragment thereof” may refer to one more, such as two of the recited antibodies or antigen binding fragments thereof. For example, in any aspect, two antibodies or antigen binding fragments thereof may be envisioned, each comprising or consisting of:
a) a first antibody or antigen binding fragment thereof having a heavy chain variable region comprising:
   a CDR1 of SEQ ID NO: 33, a CDR2 of SEQ ID NO: 34, and a CDR3 of SEQ ID NO: 35, or sequences having at least 80% identity thereto, and
   a light chain variable region comprising:
   a CDR1 of SEQ ID NO: 36, a CDR2 of SEQ ID NO: 37, and a CDR3 of SEQ ID NO: 38, or sequences having at least 80%, identity thereto; and
a second antibody or antigen binding fragment thereof having a heavy chain variable region comprising:
   a CDR1 of SEQ ID NO: 1, a CDR2 of SEQ ID NO: 2, and a CDR3 of SEQ ID NO: 3, or sequences having at least 80% identity thereto, and
   a light chain variable region comprising:
   a CDR1 of SEQ ID NO: 4, a CDR2 of SEQ ID NO: 5, and a CDR3 of SEQ ID NO: 6, or sequences having at least 80%, identity thereto; or
b) a first antibody or antigen binding fragment thereof having a heavy chain variable region comprising or consisting of SEQ ID NO: 39; and a light chain variable region comprising or consisting of SEQ ID NO: 40, or sequences having at least 80% identity thereto; and
a second antibody or antigen binding fragment thereof having a heavy chain variable region comprising or consisting of SEQ ID NO: 7; and a light chain variable region comprising or consisting of SEQ ID NO: 8, or sequences having at least 80% identity thereto; or
c) a first antibody or antigen binding fragment thereof having a heavy chain comprising or consisting of SEQ ID NO: 49; and a light chain comprising or consisting of SEQ ID NO: 50, or sequences having at least 80% identity thereto; and
a second antibody or antigen binding fragment thereof having a heavy chain comprising or consisting of SEQ ID NO: 41; and a light chain comprising or consisting of SEQ ID NO: 42, or sequences having at least 80% identity thereto.

For example, in any therapeutic application disclosed herein, and/or in any method of monitoring disclosed herein, any combination of antibodies or antigen-binding fragments may be utilised. Preferably, Ab 116 and 16 are used in combination.

In another embodiment, Ab 116 may be used in any therapeutic application disclosed herein, and Ab 16 may be used in monitoring of the same subject. Alternatively, Ab 16may be used in any therapeutic application disclosed herein, and Ab 116 may be used in monitoring of the same subject.

The term “antibody” as referred to herein refers to a glycoprotein comprising at least two heavy (H) chains and two light (L) chains inter-connected by disulfide bonds. Each heavy chain is comprised of a heavy chain variable region (V_{H}) and a heavy chain constant region. Each light chain is comprised of a light chain variable region (V_{L}) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g effector cells) and the first component (Clq) of the classical complement system.

The term “antigen-binding fragment thereof” of an antibody refers to one or more fragments of an antibody that retain the ability to selectively bind to an antigen. Antigen-binding fragments thereof may be, but are not limited to Fab, modified Fab, Fab′, modified Fab′, F(ab′)₂, Fv, single domain antibodies (e.g. VH or VL or VHH), scFv, bi, tri or tetra-valent antibodies, Bis-scFv, diabodies, triabodies, tetrabodies and epitope-binding fragments of any of the above (Holliger and Hudson, 2005, Nature Biotech. 23(9): 1126-1136; Adair and Lawson, 2005, Drug Design Reviews-Online 2(3), 209-217). The methods for creating and manufacturing these antigen-binding fragments are well known in the art (see for example Verma et al., 1998, Journal of Immunological Methods, 216, 165-181).

The antibody or antigen binding fragment thereof may be a monoclonal antibody, bispecific antibody, multi-specific antibody, ScFv or other single chain or modified format, Fab, (Fab′)2, Fv, dAb, Fd, nanobody, camelid antibody or a diabody. Preferably, the antibody or antigen binding fragment thereof is a monoclonal antibody.

The inventors have targeted CD1a and its potential role in inflammatory skin and mucosal diseases and disorders, or associated systemic diseases or disorders, or inflammatory drug reactions which manifest systemically, by generating effective monoclonal antibodies. As CD1a is highly expressed in the skin and mucosae, use of such antibodies provides an opportunity to selectively treat inflammatory skin and mucosal diseases and disorders whilst minimising off target effects. CD1a is not expressed by mice but is expressed by other mammals. Human CD1a (UniProtKB/Swiss-Prot: P06126-CD1A_HUMAN) is expressed from a dominant allele worldwide, with a variant that is present in some Chinese ethnic groups (18). Targeting CD1a antigen presentation also intercepts the inflammatory pathway upstream of other cytokine-directed antibody therapies such as anti-IL17 therapies, or other immune therapies, and therefore provides a powerful means to modulate proinflammatory disorders early in the immune cascade. Furthermore, utilising the specificity of CD1a to the skin may provide the means to direct additional therapies to the skin, for example by use of bi-specific, or multi-specific or conjugate antibody technology, to specifically target small molecule, drug, nucleic acid, peptide, antibody, or cell conjugate therapies. Further still, as CD1a is relatively non-polymorphic, the invention provides universal potential in the prevention and/or treatment of inflammatory skin and mucosal diseases such as atopic dermatitis and psoriasis, where the frequency of CD1a-expressing dendritic cell subsets is increased, and migratory patterns of LCs are altered (13-15), or CD1a-expressing malignancies.

By modifying the number and function of CD1a-expressing cells, the antibodies will have effects beyond lipid reactivity and influence all roles of CD1a-expressing cells, including antigen presentation to peptide-specific T cells and innate pathways (for example neutrophils). The antibodies of the invention are able to reduce Langerhans cells despite their murine IgG1 nature. Such reduction offers a means of controlling broad inflammatory pathways in the absence of complement/ADCC-associated inflammation, which may offer therapeutic benefit. This is shown in the imiquimod model described herein, where antibodies according the invention for example reduce inflammation including to levels significantly below the wild-type mouse, demonstrating a profound anti-inflammatory effect on pathways beyond CD1a-expressing cells, including innate pathways such as neutrophils and eosinophils. The antibodies of the invention also inhibit the production of diverse cytokines including IFN-gamma and IL-22 which are relevant to a broad range of clinical diseases.

In another aspect, the invention provides a nucleic acid encoding an antibody or antigen binding fragment thereof of the invention. Such nucleic acids may be provided by any of SEQ ID Nos: 51-90. The skilled person will understand that due to codon redundancy, a number of DNA sequences may be used to encode an antibody or antigen binding fragment thereof of the invention. Alternatively, codon optimization of the nucleotide sequence can be used to improve the efficiency of translation in expression systems for the production of an antibody or antigen binding fragment thereof of the invention.

In another aspect, the invention provides a vector comprising a nucleic acid of the invention. Suitable vectors can be chosen or constructed, containing appropriate regulatory sequences, including promoter sequences, terminator sequences, polyadenylation sequences, enhancer sequences, marker genes and other sequences as appropriate. Vectors may be for example plasmids or viral. For further details see, for example, (Sambrook, J., E. F. Fritsch, and T. Maniatis. (1989), Molecular cloning: a laboratory manual, 2nd ed. Cold Spring Harbor Laboratory, Cold Spring Harbor, New York). Many known techniques and protocols for manipulation of nucleic acid, for example in preparation of nucleic acid constructs, mutagenesis, sequencing, introduction of DNA into cells and gene expression, and analysis of proteins, are described in detail in (Ausubel et al., Current protocols in molecular biology. New York: Greene Publishing Association; Wiley-Interscience, 1992). The vector may be an expression vector. The vector or expression vector may be a plasmid.

A nucleic acid molecule or vector of the invention may be expressed using any suitable expression system, for example in a suitable host cell or in a cell-free system.

In another aspect, the invention provides a host cell comprising an antibody or antigen binding fragment thereof, nucleic acid, and/or vector of the invention. The host cell may be selected from bacterial host cells (prokaryotic systems) such as E. Coli, or eukaryotic cells such as those of yeasts, fungi, insect cells or mammalian cells. Preferably a host cell of the invention is capable of producing the antibody or antigen binding fragment thereof of the invention. The produced antibody or antigen binding fragment thereof may be enriched by means of selection and/or isolation.

An antibody or antigen binding fragment thereof of the invention may also be produced by chemical synthesis. The obtained antibody or antigen binding fragment thereof may be enriched by means of selection and/or isolation.

According to a further aspect, the invention provides a pharmaceutical composition comprising an antibody or antigen binding fragment thereof, nucleic acid, vector and/or host cell of the invention, optionally together with one or more pharmaceutically acceptable excipients or diluents.

Antibodies or antigen binding fragments thereof, nucleic acids, vectors or host cells of the invention can be formulated into pharmaceutical compositions using established methods of preparation (Gennaro, A.L. and Gennaro, A.R. (2000) Remington: The Science and Practice of Pharmacy, 20th Ed., Lippincott Williams & Wilkins, Philadelphia, PA). To prepare the pharmaceutical compositions, pharmaceutically inert inorganic or organic excipients can be used. To prepare for example pills, powders, gelatin capsules or suppositories, lactose, talc, stearic acid and its salts, fats, waxes, solid or liquid polyols, natural and hardened oils are examples of pharmaceutically acceptable excipients which can be used. Suitable excipients for the production of solutions, suspensions, emulsions, aerosol mixtures or powders for reconstitution into solutions or aerosol mixtures prior to use include water, alcohols, glycerol, polyols, and suitable mixtures thereof as well as vegetable oils.

A pharmaceutical composition of the invention may be administered via any parenteral or non-parenteral (enteral) route that is therapeutically effective. Parenteral application methods include, for example, intracutaneous, subcutaneous, intramuscular, intratracheal, intranasal, intravitreal or intravenous injection and infusion techniques, e.g. in the form of injection solutions, infusion solutions or mixtures, as well as aerosol installation and inhalation, e.g. in the form of aerosol mixtures, sprays or powders. A pharmaceutical composition of the invention can be administered systemically or topically in formulations containing conventional non-toxic pharmaceutically acceptable excipients or carriers, additives and vehicles as desired. A combination of intravenous and subcutaneous infusion and/or injection might be most convenient in case of compounds with a relatively short or long serum half-life or needing rapid onset of action. Preferably, the pharmaceutical composition is administered subcutaneously or intravenously. The pharmaceutical composition may be an aqueous solution, an oil-in water emulsion or a water-in-oil emulsion.

For intravenous injection, or injection at the site of affliction, or other site of administration, the active ingredient will be in the form of a parenterally acceptable aqueous solution which is pyrogen-free and has suitable pH, isotonicity and stability. Those of relevant skill in the art are well able to prepare suitable solutions using for example, isotonic vehicles such as Sodium Chloride Injection, Ringer's Injection, Lactated Ringer's Injection. Preservatives, stabilisers, buffers, antioxidants and/or other additives may be included, as required.

The compositions are preferably administered to an individual in a “therapeutically effective amount”, this being sufficient to show benefit to the individual. The optimal dosage will depend on the biodistribution of the antibody or antigen binding fragment thereof, the mode of administration, the severity of the disease/disorder being treated as well as the medical condition of the patient. If desired, the antibody or antigen binding fragment thereof may be given in a sustained release formulation, for example liposomal dispersions or hydrogel-based polymer microspheres, like PolyActive™ or OctoDEX™ (cf. Bos et al., Business Briefing: Pharmatech 2003: 1-6). Other sustained release formulations available are for example PLGA based polymers (PR pharmaceuticals), PLA-PEG based hydrogels (Medincell) and PEA based polymers (Medivas). Prescription of treatment, e.g., decisions on dosage etc, is within the responsibility of a medical practitioner, and typically takes account of the disorder to be treated, the condition of the individual patient, the site of delivery, the method of administration and other factors known to practitioners.

The pharmaceutical composition may also contain additives, such as, for example, fillers, binders, wetting agents, glidants, stabilizers, preservatives, emulsifiers, and furthermore solvents or solubilizers or agents for achieving a depot effect. The latter is that fusion proteins may be incorporated into slow or sustained release or targeted delivery systems, such as liposomes and microcapsules.

In another aspect, an antibody or antigen binding fragment thereof, nucleic acid, vector, host cell or pharmaceutical composition of the invention may be for use in the treatment or prevention of one or more disease or disorder in a subject.

In an aspect, there is provided a method of treating or preventing one or more disease or disorder in a subject, comprising administering to the subject an effective amount of an antibody or antigen binding fragment thereof, nucleic acid, vector, host cell or composition of the invention.

In an aspect, there is provided the use of an antibody or antigen binding fragment thereof, nucleic acid, vector, host cell or pharmaceutical composition of the invention in the manufacture of a medicament for the treatment or prevention of one or more diseases or disorders in a subject.

In any aspect, the subject may be a mammal. The mammal may express a CD1a orthologue. Preferably, the subject is a human.

The one or more disease or disorder may be one or more inflammatory skin or mucosal disorder, or disease or one or more associated systemic disease or disorder, or one or more inflammatory drug reaction which manifests systemically, or a CD1a-expressing malignancy.

An inflammatory skin or mucosal disease or disorder may be selected from:
a) a predominantly neutrophilic skin disease, such as acne, generalized pustular psoriasis, plaque psoriasis, guttate psoriasis, palmoplantar pustulosis, SAPHO syndrome, acute febrile neutrophilic dermatosis (Sweet syndrome), histiocytoid neutrophilic dermatitis, neutrophilic dermatosis of the dorsal hands, pyoderma gangrenosum, neutrophilic eccrine hidradenitis, hidradenitis suppurativa, erythema elevatum diutinum, Behcet disease, bowel-associated dermatitis-arthritis syndrome, other infection-associated inflammation, neutrophilic urticarial dermatosis, palisading neutrophilic granulomatous dermatitis, erythema gyratum repens, neutrophilic annular erythema, acute generalised exanthematous pustulosis (AGEP), vasculitis, and others;
b) an autoimmune disorder, such as connective tissue disease (eg lupus, dermatomyositis, scleroderma/systemic sclerosis, Churg Strauss syndrome), panniculitis, vasculitides, autoimmune blistering conditions (eg bullous pemphigoid, pemphigus, linear IgA disease), dermatitis herpetiformis, coeliac disease, some auto-inflammatory disease, vitiligo, alopecia areata, alopecia universalis, alopecia totalis, panniculitis, lichen planus, erythema multiforme, lichen sclerosis, other lichenoid and erythema multiforme-like diseases, vesiculation psoriatic arthritis, rheumatoid arthritis, inflammatory bowel disease, multiple sclerosis, Guillain-Barre syndrome, thyroiditis, transverse myelitis, neurodegeneration and others;
c) mast cell disorders and eosinophilic disorders, such as Muckle Wells syndrome, eosinophilia and systemic symptoms syndrome, urticaria, angioedema, keratoconjunctivitis, food allergy, other allergy or atopy including atopic dermatitis, rhinitis, conjunctivitis, asthma, eosinophilic oesophagitis and other eosinophilic mucosal diseases, contact dermatitis and others.
d) adverse drug reactions which manifest as an inflammatory skin or mucosal disease or disorder, such as Stevens Johnsons syndrome, toxic epidermal necrolysis, drug reaction with eosinophilia and systemic symptoms syndrome (DRESS) and acute generalised exanthematous pustulosis (AGEP), erythema multiforme, bullous, fixed drug eruption, and others.
e) Graft vs host disease

A CD1a-expressing malignancy as referred to herein may be any malignancy where CD1a expression can be detected. Such malignancies may include Langerhans cell histiocytosis, subsets of T cell lymphomas, subsets of thymomas or rarely-occurring instances of other malignancies, such as subsets of mastocytosis. Preferably, the CD1a-expressing malignancy is subsets of T cell lymphomas.

Preferably the one or more disease or disorder comprises or consists of psoriasis, dermatitis, lupus erythematosus, neutrophilic dermatoses, an associated systemic disease or disorder, and/or or an inflammatory drug reaction which manifests systemically, or a CD1a-expressing malignancy.

An associated systemic disease or disorder as used herein may refer to any non-cutaneous site involvement that may be associated with an inflammatory skin or mucosal disease or disorder as defined herein. This may include non-cutaneous lupus erythematosus.

An inflammatory drug reaction which manifests systemically, may be at a non-cutaneous site such as the spleen. An associated systemic disease or disorder, or inflammatory drug reaction which manifests systemically, may be as a result of an inflammatory response. The inflammatory response may be for example to a drug such as Aldara (5% imiquimod cream). The inflammatory response may result in increased numbers or activity of CD4 T-cells, CD8 T-cells, neutrophils or eosinophils, and/or increased levels of IL-23, IL-12, IL-1B and/or MCP-1, and/or decreased IL-10 and/or IL-27.

Furthermore, an antibody or antigen binding fragment thereof, nucleic acid, vector, host cell or pharmaceutical composition of the invention may be administered alone or in combination with one or more other therapeutic agent, either simultaneously, sequentially or separately, dependent upon the condition to be treated. The one or more other therapeutic agent may be selected from the group comprising cytotoxic agents, immune activation agents such as checkpoint inhibitors or TLR agonists, anti-inflammatory agents such as steroids, CAR-T cells such as regulatory or cytolytic CAR-T cells, or other cells expressing or presenting one or more antibody or antigen binding fragment of the invention.

In another aspect, there is provided a method of monitoring treatment efficacy or disease status in a subject diagnosed with a CD1a-expressing malignancy, comprising:
i. providing a biological sample obtained from the subject;
ii. determining the level of binding of one or more antibodies or antigen binding fragments of the invention to CD1a-expressing cells in the sample obtained from the subject before treatment, or at intervals between treatments, or at time intervals in the absence of treatment;
iii. determining that the treatment is effective, or that the disease status is improving, if the tumour volume, or level of binding of one or more antibodies or antigen binding fragments of the invention to CD1a-expressing cells, is reduced after treatment or between treatment intervals or at time intervals in the absence of treatment,

A biological sample may be a blood or serum sample, tissue biopsy, cerebrospinal fluid, saliva, or urine sample. Preferably, the biological sample may be a blood or serum sample.

The level of binding of one or more antibodies or antigen binding fragments of the invention to CD1a-expressing cells in the sample may be determined using any method known to the skilled person. One such method is for example using flow cytometry or any other technique utilising a detectable label, to be able to determine the number of CD1a expressing cells in the sample.

Tumour volume may be determined by any suitable technique known to the skilled person.

The reduction in tumour volume or level of binding of one or more antibodies or antigen binding fragments of the invention to CD1a-expressing cells may be by 10% or more, such as 25% or more, 50% or more, 75% or more, or 90% or more.

The treatment intervals or time intervals in the absence of treatment may be two weeks or more, such as four weeks or more, 8 weeks or more, 12 weeks or more, six months or more, or 12 months or more.

Techniques for the production of antibodies and antigen binding fragments thereof are well known in the art. The term “antibody” also includes immunoglobulins (Ig's) of different classes (i.e. IgA, IgG, IgM, IgD and IgE) and subclasses (such as IgG1, lgG2 etc.). Illustrative examples of an antibodies or antigen binding fragments thereof include Fab fragments, F(ab′)2, Fv fragments, single-chain Fv fragments (scFv), diabodies, domain antibodies or bispecific antibodies (Holt LJ et al., Trends Biotechnol. 21(11), 2003, 484-490). Examples also include a dAB fragment which consists of a single CH domain or VL domain which alone is capable of binding an antigen. An antibody or antigen binding fragment thereof may be chimeric, a nanobody, single chain and/or humanized. The antibody or antigen binding fragment thereof may be a human IgG1 isotype or a human IgG4 isotype or other natural or modified isotype. Antibodies may be monoclonal (mAb) or polyclonal.

The antibody or antigen binding fragment thereof may be modified to change in vivo stability and/or half-life. The modification for example may be PEGylation.

The antibody or antigen binding fragment thereof may be an antibody-like molecule which includes the use of CDRs separately or in combination in synthetic molecules such as SMIPs and small antibody mimetics.

The percent identity of two amino acid sequences or of two nucleic acid sequences is generally determined by aligning the sequences for optimal comparison purposes (e.g., gaps can be introduced in the first sequence for best alignment with the second sequence) and comparing the amino acid residues or nucleotides at corresponding positions. The “best alignment” is an alignment of two sequences that results in the highest percent identity. The percent identity is determined by comparing the number of identical amino acid residues or nucleotides within the sequences (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul, 1990, PNAS, 87(6):2264-8, modified as in Karlin and Altschul, 1993, PNAS, 90(12):5873-5877 The NBLAST and XBLAST programs of Altschul et al., 1990, J. Mol. Biol., 215:403-10 have incorporated such an algorithm. BLAST nucleotide searches can be performed with the NBLAST program, score=100, word length=12 to obtain nucleotide sequences homologous to a nucleic acid molecules of the invention. BLAST protein searches can be performed with the XBLAST program, score=50, word length=3 to obtain amino acid sequences homologous to a protein molecules of the invention. To obtain gapped alignments for comparison purposes, Gapped BLAST can be utilized as described in Altschul et al. (1997). Alternatively, PSI-Blast can be used to perform an iterated search that detects distant relationships between molecules (Id.). When utilizing BLAST, GappedBLAST, and PSI-Blast programs, the default parameters of the respective programs (e.g., XBLAST and NBLAST) can be used. See http://www.ncbi.nlm.nih.gov. Another example of a mathematical algorithm utilized for the comparison of sequences is the algorithm of Myers and Miller. The ALIGN program (version 2.0) which is part of the GCG sequence alignment software package has incorporated such an algorithm. Other algorithms for sequence analysis known in the art include ADVANCE and ADAM as described in Torellis and Robotti (1994); and FASTA described in Pearson and Lipman (1988). Within FASTA, ktup is a control option that sets the sensitivity and speed of the search.

An antibody or antigen binding fragment thereof of the invention may comprise one or more mutated amino acid residues. The terms “mutated”, “mutant” and “mutation” in reference to a nucleic acid or an antibody or antigen binding fragment thereof of the invention refers to the substitution, deletion, or insertion of one or more nucleotides or amino acids, respectively, compared to the “naturally” occurring nucleic acid or polypeptide, i.e. to a reference sequence that can be taken to define the wild-type.

The amino acid variations in the CDR sequences may be conservative amino acid substitutions.

A mutation may be a substitution wherein the substitution is a conservative substitution. Conservative substitutions are generally the following substitutions, listed according to the amino acid to be mutated, each followed by one or more replacement(s) that can be taken to be conservative: Ala→Gly, Ser, Val; Arg→Lys; Asn→Gln, His; Asp→Glu; Cys→Ser; Gln→Asn; Glu→Asp; Gly→Ala; His→Arg, Asn, Gln; Ile→Leu, Val; Leu→Ile, Val; Lys→Arg, Gln, Glu; Met→Leu, Tyr, He; Phe→Met, Leu, Tyr; Ser→Thr; Thr→Ser; Trp→Tyr; Tyr→Trp, Phe; Val→He, Leu. Other substitutions are also permissible and can be determined empirically or in accord with other known conservative or non-conservative substitutions.

1, 2 or 3 conservative substitutions may be made in the CDRs of the antibody or antigen binding fragment thereof of the invention.

Methods of making an antibody or antigen binding fragment thereof are well known in the art. The skilled person may use hybridoma technology for example, or may use recombinant DNA technology to clone the respective antibody sequence into a vector, such as an expression vector. Methods of making a bispecific antibody molecule are known in the art, e.g. recombinant DNA technology, chemical conjugation of two different monoclonal antibodies or for example, also chemical conjugation of two antibody fragments, for example, of two Fab fragments. Alternatively, bispecific antibody molecules are made by quadroma technology, which is by fusion of the hybridomas producing the parental antibodies. Because of the random assortment of H and L chains, a potential mixture of ten different antibody structures are produced of which only one has the desired binding specificity. A bispecific antibody molecule of the invention can act as a monoclonal antibody (mAb) with respect to each target. The antibody or antigen binding fragment thereof may be chimeric, humanized or fully human. The antibody or antigen binding fragment thereof may be a human IgG1 isotype or a human IgG4 isotype or other natural or modified isotype. A bispecific antibody molecule or multi-specific antibody may for example be a bispecific tandem single chain Fv, a bispecific Fab2, or a bispecific diabody.

All of the features disclosed in this specification may be combined in any combination, including with any aspect or any embodiment.

### BRIEF DESCRIPTION OF THE FIGURES

FIG. 1—shows the inhibition of polyclonal T cell responses by a panel of anti-CD1a antibodies. A. Dose titration curve of polyclonal T cell IFNγ response with increasing concentration of anti-CD1a antibody (0.01-10 μg/ml) (n=6 donors). B. IC50 values calculated for the panel of newly generated anti-CD1a antibodies and commercial antibodies (OKT6, HI149 and SK9, n=6 donors)

FIG. 2—demonstrates the inhibition of CD1a-restricted enriched T cell line responses by a panel of anti-CD1a antibodies. A-B. Cytokine secretion response of CD1a-restricted enriched T cell lines induced by empty vector (EV) or CD1a transfected K562 presenting endogenous ligands. Inhibition of IFNγ (A.) or IL-22 (B.) was assessed for the panel of newly generated anti-CD1a antibodies by flow cytometry. C. IFNγ secretion response of CD1a-restricted enriched T cell lines induced by CD1a coated beads presenting endogenous ligands. Inhibition was assessed for the panel of newly generated anti-CD1a antibodies by flow cytometry. Inhibition was assessed for the panel of newly generated anti-CD1a antibodies by flow cytometry. (N=4-19 enriched T cell lines, 2-way-ANOVA with Tukey's test, *, P<0.05; **, P<0.01; ***, P<0.001; ****, P<0.0001 where * indicates significance on comparison to “CD1a”.

FIG. 3—demonstrates the characterisation of CD1a transgenic mouse. A. Representative flow cytometry plots and B. graphical summary of CD1a protein expression by cells of wild-type (WT) and CD1a transgenic (CD1a) mice. CD1a protein expression evaluated on (left-right) total live car skin cells, CD45+ skin cells, dermal dendritic cells (dDCs, CD45+/CD11c+/langerin−) and Langerhans cells (LCs, CD45+/CD11c+/langerin+). C. CD1a protein expression within car skin of wild-type (WT) and CD1a transgenic (CD1a) mice, visualised by immunofluorescence. Cryosections were stained with DAPI (blue) and anti-CD1a AF-594 (OKT6, red), scale bars left to right 50 μm, 50 μm and 10 μm. D. Exemplar PCR genotyping of CD1a transgenic mouse line litter (lanes A-F) using CD1a forward and reverse primers and tail genomic DNA. Expected CD1a band at 655 bp. Lane G: positive control genomic DNA from founder mouse. Lane H: negative control lacking DNA template. E Representative flow cytometry plots of thymic CD1a protein expression by wild-type (WT) and CD1a transgenic (CD1a) mice.

FIG. 4—Characterisation of anti-CD1a antibodies in vivo. A. Schematic of imiquimod-induced skin inflammation and anti-CD1a preventative administration. B. Daily measurement of ear swelling induced by imiquimod treatment of wild-type (WT) and CD1a transgenic mice (CD1a) injected i.p. with mouse IgG1 isotype control and CD1a transgenic injected with the refined panel of anti-CD1a antibodies as in the schematic panel A. (N=6, 2-way-ANOVA with Dunnett's test, **, P<0.01; ****, P<0.0001 indicates significance on comparison to “CD1a” at day 6 or as shown).

FIG. 5—demonstrates the effect of anti-CD1a on the imiquimod-induced cutaneous immune response. A-C. Flow cytometric analysis of ear skin of mouse IgG1 isotype treated wildtype (WT) and CD1a transgenic (CD1a) and CD1a transgenic injected with the refined panel of anti-CD1a antibodies following the preventative model of administration. Skin T cells were enumerated (A.) and assessed for cell surface CD69 expression (B.) and skin neutrophil (C.) and cosinophil (D.) frequency was determined. (N=4, 1-way-ANOVA with Dunnett's test, *, P<0.05; **, P<0.01; ***, P<0.001).

FIG. 6—demonstrates the effect of anti-CD1a on the imiquimod-induced cellular Langerhans cell skin and lymph node response. Flow cytometric analysis of car skin (A-B.) and draining cervical lymph node (C-D.) of mouse IgG1 isotype treated wildtype (WT) and CD1a transgenic (CD1a) and CD1a transgenic injected with the refined panel of anti-CD1a antibodies following the preventative model of administration. Skin LCs were enumerated (A.) and assessed for cell surface CD1a expression (B.). Lymph node LCs were enumerated (C.) and assessed for cell surface CD1a expression (D.). (N=4, 1-way-ANOVA with Dunnett's test, *, P<0.05; **, P<0.01; ***, P<0.001; ****, P<0.0001).

FIG. 7—demonstrates antibody dependent depletion (phenotypic change). A. Flow cytometric analysis of antibody induced CD1a dependent cell reduction (such as death). Anti-CD1a antibodies or mouse IgG1 isotype control (iso, 5 μg/ml) were incubated with EV or CD1a-K562 as indicated for 48 hours and percentage of antibody induced reduction was calculated in relation to a reference population of untreated K562 and was normalised to EV control cells. B. Dose titration curve of antibody induced CD1a-K562 cell reduction with increasing concentration of anti-CD1a antibody (0.625-5 μg/ml). C-D. Anti-CD1a antibodies or mouse IgG1 isotype control (iso, 5 μg/ml) were incubated with MoDCs (upper panel) and MoLCs (lower panel) as indicated for 5 days with antibodies and cytokines added on day 0 or day 2 and percentage of antibody induced reduction was calculated in relation the isotype control as measured by percentage confluence using Incucyte live cell imaging (N=4, 2-way-ANOVA with Tukey's test.) (C.) and representative images of MoLCs (D.). E. K562-CD1a or K562-EV (empty vector) were incubated with anti-CD1a antibodies for 24 hours and stained for Annexin V and analysed by flow cytometry. (N=3-4, 1-way-ANOVA with Tukey's test.) F. Flow cytometric analysis of complement-dependent cytotoxicity (CDC). K562-CD1a cells were incubated with 10% normal human serum for 3-hours at 37° C. in the presence of either 5 μg/ml isotype control antibody or indicated antibodies. Percentage cytotoxicity was calculated in relation to a reference population of untreated K562 and was normalised to isotype control treated cells. (N=6, 1-way-ANOVA with Tukey's test.) G. Flow cytometric analysis of antibody-dependent cell-mediated cytotoxicity (ADCC). K562-CD1a cells were co-cultured with PBMC at 1:50 ratio for 5-hour at 37° C. in the presence of either 5 μg/ml isotype control antibody or indicated antibodies. Percentage cytotoxicity was calculated in relation to a reference population of untreated K562 and was normalised to isotype control treated cells. (N=4-6, 1-way-ANOVA with Tukey's test.) H. NSG mice were subcutaneously injected with 0.25 million CD1a-K562 cells in the flank and tumours were allowed to develop for 18 days. Mice were treated with 100 μg isotype control antibody or indicated antibodies on days 6, 10, and 14 intraperitoneally. Measurement of tumour volume over time. (N=6-15, 2-way-ANOVA with Tukey's test, asterisks indicate significance on comparison to “CD1a-iso” at day 18).*, P<0.05; **, P<0.01; ***, P<0.001; ****, P<0.0001.

FIG. 8 (A)—is a heatmap from CD1a epitope analysis. Matrix heatmap representation of CD1a antibody binding by flow cytometry as measured by CD1a-AF647 mean fluorescence intensity (MFI). Before staining of CD1a-K652 with anti-CD1a antibodies conjugated to fluorophore AF647, the relevant purified antibodies were incubated with the cells to assess interference in CD1a binding of the AF647-conjugated antibodies. Grayscale shows degree of interference with the tone in the top row (−) indicating no interference. (B)—demonstrates in vivo CD1a antibody epitope competition assay results. A. Flow cytometry plots of CD1a expression as measured by staining with anti-CD1a antibodies SK9 (left panels) or HI149 (right panels). Anti-CD1a antibody 116 (100 μg i.p.) was administered on days 0, 2 and 4 and car skin tissue collected, processed and stained for CD1a on day 5.

FIG. 9—demonstrates the effectiveness of application of anti-CD1a antibodies in the treatment of imiquimod-induced inflammation. A. Schematic of imiquimod-induced inflammation model with therapeutic anti-CD1a administration. B. Daily measurement of ear swelling and C. representative images of inflammation (day 8) induced by imiquimod treatment of wild-type (WT) and CD1a transgenic mice (CD1a) followed by the treatment i.p. with mouse IgG1 isotype control or CD1a transgenic injected with the refined panel of anti-CD1a antibodies as in the schematic panel A (at day 3 arrowpoint) (N=2-10, 2-way-ANOVA with Dunnett's test, **, P<0.01; ****, P<0.0001 indicates significance on comparison to “CD1a” at day 8 or as shown). D. Ear and epidermal thickness and CD1a protein expression within car skin of wild-type (WT) and CD1a transgenic (CD1a) mice treated with imiquimod (Imiq) or untreated (U) visualised by immunofluorescence. Cryosections were stained with DAPI (blue) and anti-CD1a AF-594 (OKT6, red), scale bars 10 μm upper panels and 100 μm lower panels. E-G. Flow cytometric analysis of ear skin of mouse IgG1 isotype treated wild-type (WT) and CD1a transgenic (CD1a) and CD1a transgenic injected with the refined panel of anti-CD1a antibodies following the treatment model of administration. Skin T cells were enumerated and assessed for cell surface CD11a expression (E.) and neutrophil (F.) and cosinophil (G.) frequency was determined. (N=7-9, 1-way-ANOVA with Dunnett's test, *, P<0.05; **, P<0.01; ***, P<0.001).

FIG. 10—demonstrates the CD1a dependency of the systemic effects of imiquimod application. A. Spleen weight (mg) measurements and representative images on day 8 by imiquimod treatment of wild-type (WT) and CD1a transgenic mice (CD1a) followed by treatment i.p. with mouse IgG1 isotype control or CD1a transgenic injected with the refined panel of anti-CD1a antibodies as in the schematic (FIG. 9A). B-E. Flow cytometric analysis of spleen of mouse IgG1 isotype treated wild-type (WT) and CD1a transgenic (CD1a); and CD1a transgenic injected with the refined panel of anti-CD1a antibodies following the treatment model of administration. Splenic CD4 (B.) and CD8 (C.) T cell CD69 expression was assessed and neutrophils (D.) and eosinophils (E.) were enumerated. (N=7-9, 1-way-ANOVA with Dunnett's test, *, P<0.05; **, P<0.01; ***, P<0.001; ****, P<0.0001). F. Plasma cytokine levels of the blood of mouse IgG1 isotype treated wild-type (WT) and CD1a transgenic (CD1a); and CD1a transgenic injected with anti-CD1a antibodies following the treatment model of administration (N=7-9, 1-way-ANOVA with Dunnett's test, *, P<0.05; **, P<0.01; ***, P<0.001; ****, P<0.0001).

FIG. 11—demonstrates CD1a dependency of the systemic effects of imiquimod application. A-E. Blood cellular analysis of the blood of mouse IgG1 isotype treated wild-type (WT) and CD1a transgenic (CD1a); and CD1a transgenic injected with the refined panel of anti-CD1a antibodies following the treatment model of administration. Circulating T cells (A.), CD4+ (B.) and CD8+ (C.), neutrophils (D.) and eosinophils (E.) were enumerated. (N=5-7, 1-way-ANOVA with Dunnett's test, *, P<0.05; **, P<0.01; ***, P<0.001; ****, P<0.0001).

FIG. 12—shows that imiquimod does not constitute a CD1a ligand. Isoelectric point dependent migration of mock and imiquimod “loaded” CD1a protein on isoelectric focusing (IEF) gel pH3-7. Mock: vehicle control TBS 2% CHAPS 7% DMSO.

FIG. 13—effectiveness of application of anti-CD1a antibodies in sustained control of imiquimod-induced inflammation. A. Schematic of imiquimod re-challenge model without later anti-CD1a administration. B. Daily measurement of ear swelling induced by imiquimod treatment of wild-type (WT) and CD1a transgenic mice (CD1a) injected i.p. with mouse IgG1 isotype control and CD1a transgenic injected with the refined panel of anti-CD1a antibodies as in the schematic panel 13A (2-way-ANOVA with Dunnett's test, *, P<0.05; **, P<0.01 indicates significance on comparison to “CD1a” isotype at day 7 of imiquimod re-application).

FIG. 14—effectiveness of application of anti-CD1a antibodies in treatment of imiquimod-induced inflammation, compared to a standard of care. Daily measurement of car swelling induced by imiquimod treatment of wild-type (WT) and CD1a transgenic mice (CD1a) followed by the treatment i.p. with mouse IgG1 isotype control (CD1a) or CD1a transgenic injected with the refined panel of anti-CD1a antibodies and anti-IL-17A as in the schematic panel FIG. 9A. dx=day of model that significance was reached compared to CD1a transgenic car thickness.

FIG. 15—comparator analysis of the effectiveness of application of anti-CD1a antibodies in the treatment of imiquimod/MC903-induced inflammation. A. Schematic of imiquimod-induced inflammation with therapeutic anti-CD1a administration. B. Daily measurement of ear swelling induced by imiquimod treatment of wild-type (WT) and CD1a transgenic mice (CD1a) followed by the treatment i.p. with mouse IgG1 isotype control or CD1a transgenic injected with the refined panel of anti-CD1a antibodies or CR2113 as in the schematic panel A. N=2-7, 2-way-ANOVA with Dunnett's test, *, P<0.05, **, P<0.01; ****, P<0.0001 indicates significance on comparison to “CD1a” at day 8 or OX116 vs CR2113 at day 8. C. Data and comparisons presented in (B), corrected for WT. D. Schematic of MC903-induced inflammation with preventative MC903-induced inflammation. E. Daily measurement of car swelling induced by MC903 treatment of wild-type (WT) and CD1a transgenic mice (CD1a) after the treatment i.p. with mouse IgG1 isotype control or CD1a transgenic injected with 16, 110 or 116 anti-CD1a antibody or CR2113 as in the schematic panel D. Corrected for WT. N=3-4, 2-way-ANOVA with Dunnett's test, *, P<0.05, indicates significance on comparison to “CD1a” at day 7. F. Skin T cell percentage and cosinophil count measured by flow cytometry. N=3-4, 2-way-ANOVA with Dunnett's test, *, P<0.05; **, P<0.01; ***, P<0.001.

FIG. 16—comparator analysis of the effect of anti-CD1a antibodies in skin and systemic immune responses with imiquimod-induced inflammation. Ear skin, draining cervical lymph node and plasma samples were analysed from mouse IgG1 isotype treated wildtype (WT) and CD1a transgenic (CD1a) and CD1a transgenic injected with the refined panel of anti-CD1a antibodies following the treatment model of administration as shown in schematic FIG. 15A. A. Skin T cell IL-17A expression was analysed using intracellular cytokine expression detected by flow cytometry directly ex vivo (left panel), and cervical lymph node eosinophils were enumerated (right panel). B-C. Plasma (B) and skin digest (C) cytokine levels were measured by ELISA (N=2-7, 1-way-ANOVA with Dunnett's test, *, P<0.05; **, P<0.01; ***, P<0.001; ****, P<0.0001).

### Materials and Methods

### Mice

All mice were bred in a specific pathogen-free facility. In individual experiments, mice were matched for age, sex and background strain with wild-type litter mates used as matched controls. All experiments undertaken in this study were done so with the approval of the UK Home Office.

### CD1a Transgenic Mouse Generation

Mice were generated by the Wellcome Trust Centre for Human Genetics, Oxford. A 5.7 kb genomic fragment encompassing the entire CD1A gene, including 0.8 kb of upstream sequence and 0.8 kb of downstream sequence, was amplified from human genomic DNA by PCR using primers 5′-ATGGTACCAAGAGGAATGTAAATGTGTCCGGC-3′ and 5′-AAGCGGCCGCGATCATGTTAACCAAGGTCAGGAA-3′ and subcloned into the Litmus28 vector (NEB) via the KpnI and NotI sites incorporated into these PCR primers. After sequence verification of the coding exons, the fragment transgene was excised from the vector backbone, purified and resuspended at 2 ng/ul in microinjection buffer (10 mM Tris-HCl, pH 7.4, 0.25 mM EDTA) and microinjected into a pronucleus of fertilized zygotes prepared from C57BL/6J mice. After overnight culture, the resulting 2-cell embryos were surgically implanted into the oviduct of pseudopregnant CD1 foster mother and carried to term. Transgenic offspring were identified by PCR using transgene specific primers and bred as individual lines with wild-type C57BL/6J mice.

### CD1a Genotyping

Crude genomic DNA preparation was performed on ear notch samples from CD1a transgenic mice. 100 μl of DirectPCR ear lysis buffer (Viagen) supplemented with 0.4 mg/ml proteinase K (Sigma) was added to ear notches and incubated at 55° C. overnight. Enzymes were then heat inactivated at 85° C. for 1 hour. The samples were centrifuged to pellet debris and the lysate was transferred to a clean tube. 1 μl of lysate was used as a template for genotyping. The below PCR reaction was used for genotyping. PCR products were loaded on to a 1% TAE agarose gel with SyberSafe, electrophoresis run and the gel imaged under UV. If the expected band at 655 bp was detected, mice were considered positive for the CD1a transgene.

| | |
|---|---|
| DNA template | 1 ul |
| MyTaqRed Mix (BioLine) 2x | 25 ul |
| CD1a forward primer | 0.4 μM |
| CD1a reverse primer | 0.4 μM |
| dd-H20 | Up to 50 ul reaction volume |

| | |
|---|---|
| 1. Initial denaturation | 95° C., 2 min |
| Cycle: Steps 2-4 | X34 |
| 2. Denaturation | 95° C., 20 sec |
| 3. Annealing | 60° C., 15 sec |
| 4. Extension | 72° C., 20 sec |
| 5. Final extension | 72° C., 2 mins |
| 6. Hold | 4° C. |

### Cell Lines

Empty vector-transfected K562 (EV-K562) and CD1a-transfected K562 (CD1a-K562) cells (a gift from B. Moody, Brigham and Women's Hospital, Harvard Medical School, Boston, MA) were maintained in RPMI 1640 medium supplemented with 10% FCS, 100 IU/ml penicillin, 100 μg/ml streptomycin (Sigma-Aldrich), 2 mM L-glutamine (Gibco), 1×nonessential amino acids (NEAAs) (Gibco), 1 mM sodium pyruvate (Gibco), 10 mM HEPES (Gibco), 500 μM 2-mercaptoethanol (Gibco), and 200 μg/ml G418 antibiotic (Thermo Fisher Scientific).

### ELISpot Analysis

ELISpot assay was used to detect activation-induced cytokine secretion from polyclonal T cells upon coculture with model CD1a expressing antigen presenting cells. PBMCs from healthy donor blood were isolated by density gradient (Lymphoprep) and T cells purified using anti-CD3 magnetic bead sorting following the manufacturer's protocol (MACS, Miltenyi). All study participants gave fully informed written consent [National Health Service (NHS) National Research Ethics Service (NRES) research ethics committee 14/SC/0106. T cells were then cultured for 3 days with IL-2 (200 U/ml) to expand in number prior to overnight co-culture with unpulsed/endogenous lipid bound CD1a-transfected K562 (CD1a-K562) or control empty-vector transfected K562 (EV-K562) at a ratio of 25000 K562 to 50000 polyclonal T cells. To assess the functionality of the anti-CD1a antibodies, K562 were incubated with 10 μg/ml anti-CD1a antibodies 1 hour prior to and during co-culture with polyclonal T cells in an anti-IFNγ capture antibody coated ELISpot plate. IFNγ secretion was detected with a biotinylated anti-IFNγ detection antibody and visualised with streptavidin-alkaline phosphatase development. Resulting spots were indicative of cytokine producing T cells and were enumerated using an automated ELISpot reader (Autimmun Diagnostika gmbh ELISpot Reader Classic), and the % blockade was calculated upon comparison of the antibody treated and untreated groups following subtraction of the EV background level of cytokine production spots. The EV-K562 contribution (with and without antibody) was subtracted from the CD1a IFNγ spot number (with and without antibody respectively). The adjusted CD1a-K562 antibody-treated group spot number was then divided by the CD1a without antibody group and used to calculate % blockade.

### CD1a-Reactive T Cell Generation and Activation Analysis

CD1a-restricted T cells were isolated by fluorescence activated cell sorting. T cells were co-cultured with EV-K562 of CD1a-K562 and cytokine producing responder T cells were detected using Miltenyi MACS Cytokine Secretion assays following the manufacturer's instructions. Briefly T cells were coated with anti-cytokine (IL-22 or IFNγ) antibody after a 6-hour culture with CD1a-K562 to detect CD1a dependent autocrine cytokine production. The live responder cells were then sorted into a culture plate. CD1a-restricted T cells were expanded with mixed lymphocyte reaction, and purity and CD1a-responsiveness were assessed with the above FACS-based cytokine secretion assay method using an analysing flow cytometer. The activation of CD1a-restricted T cells was analysed as follows. 2×10⁵ K562 cells were co-cultured with 1-5×10⁵ CD1a-autoreactive T cells for 4 hr. Helper cytokines were added to the co-culture to support CD1a-dependent cytokine production. IFNγ-producing T cell culture was supplied with IL-12 (1 ng/ml, BioLegend), IL-18 (1 ng/mL, BioLegend), and IL-2 (25 U/mL, BioLegend); and IL-22-producing T cell culture were supplied with IL-6 (5 ng/ml, BioLegend), TNF-γ (5 ng/ml, BioLegend), and IL-2 (25 U/mL, BioLegend). Activation of T cells was assessed by cytokine production of T cells using the above secretion Aasay (Miltenyi Biotec) following the manufacturer's instructions.

### Murine Imiquimod Administration

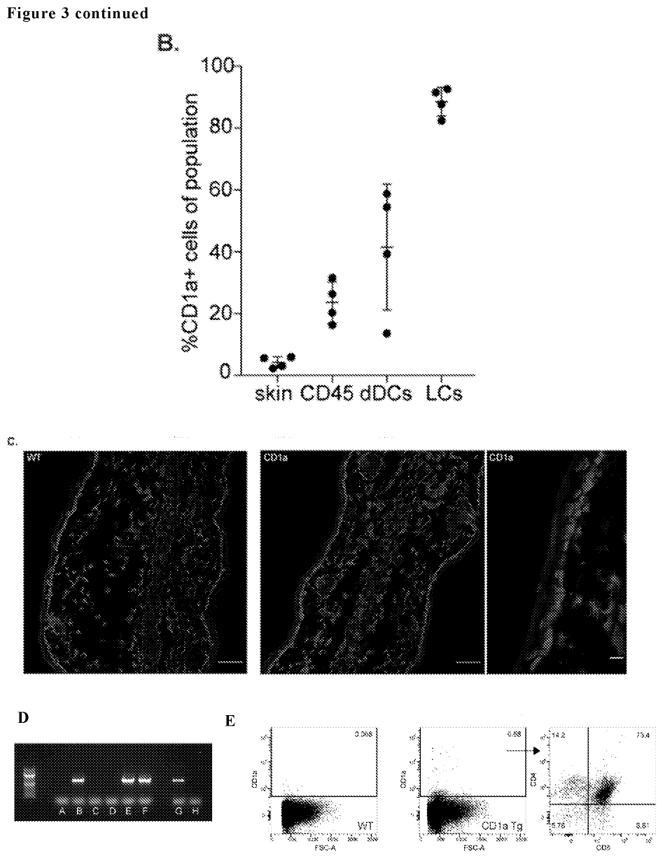

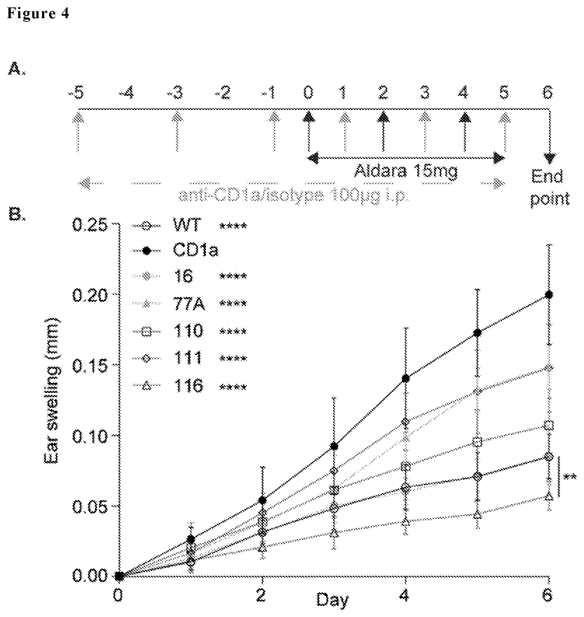

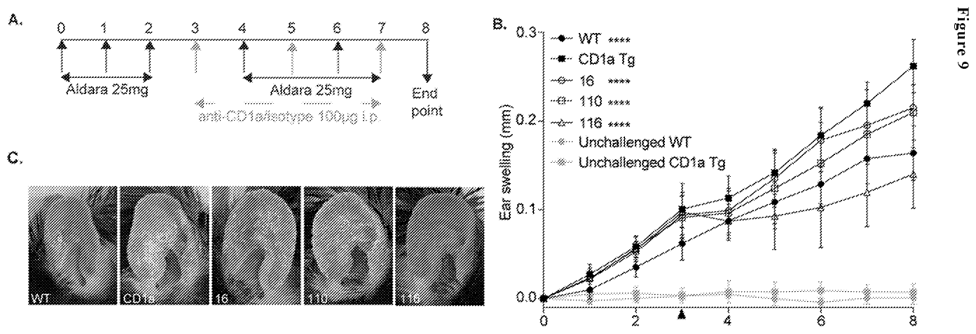

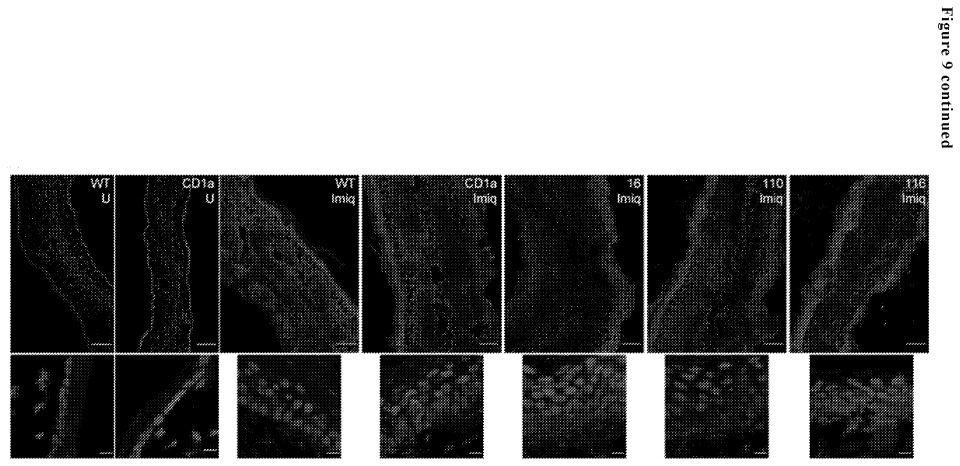

Mice were lightly anaesthetised with isoflurane and 15 mg Aldara cream containing 5% imiquimod was applied to the dorsal and ventral sides of the ear pinnae on days 0, 1, 2, 3, 4, 5 in the prevention model (FIG. 4A) or 0, 1, 2 and 4, 5, 6, 7 in the treatment model (FIG. 9A). 100 μg anti-CD1a antibodies or mouse IgG1 isotype control were administered intraperitoneally on days −5, −3, −1, 1, 3, 5 in the prevention model (FIG. 4A) or 3, 5, 7 in the treatment model (FIG. 9A). Ear thickness measurements were taken daily throughout the duration of Aldara application days 0-6 in the prevention model (FIG. 4A) or 0-8 in the treatment model (FIG. 9A) using a micrometer (Mitutoyo). Mice were sacrificed and tissues taken 24 h after challenge.

### Murine MC903 Administration

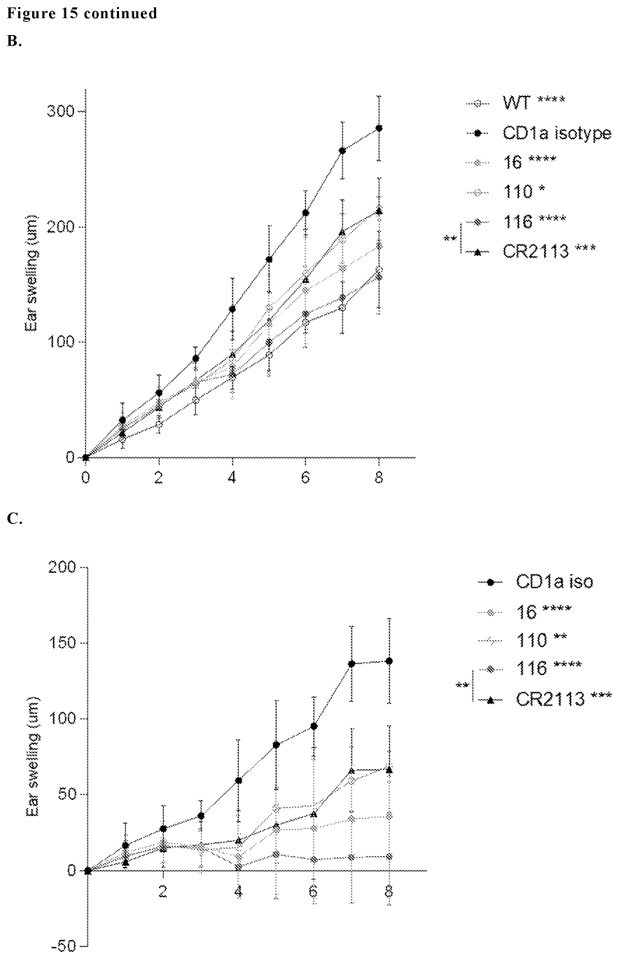

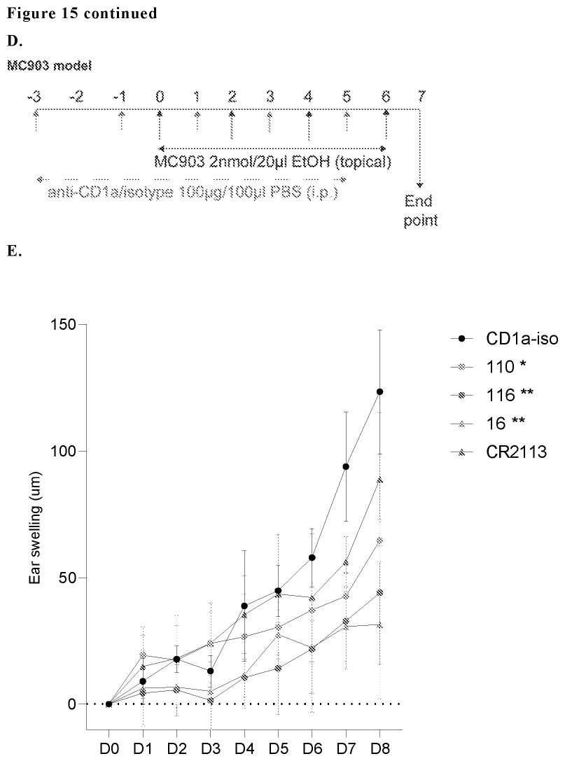

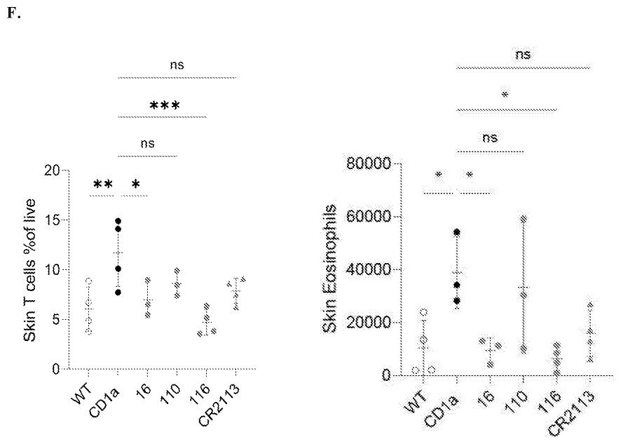

Mice were lightly anaesthetised with isoflurane and 2 nmol per dose of MC903 daily for 7 days applied to ventral and dorsal side of ear (10 microlitres each side of the ear). 100 μg anti-CD1a antibodies or mouse IgG1 isotype control were administered intraperitoneally as indicated in FIG. 15D. Ear thickness measurements were taken daily using a micrometer (Mitutoyo).

### Tissue Processing

Mice were sacrificed and tissues taken 24 h after final imiquimod challenge. Ears, cervical lymph nodes (cLN) and spleen were collected for immunophenotyping or imaging. Cell suspensions of spleen and cLN, were obtained by passing the tissues through a 70 μm strainer and washed with RPMI containing 10% FCS. Spleen cell suspension red blood cells were removed by incubation with RBC lysis solution (eBioscience).

Ear skin tissue was washed in HBSS to remove excess imiquimod, split ventrally, diced into <0.5 mm pieces and digested with 1 mg/mL collagenase P (Roche) and 0.1 mg/mL DNaseI (Sigma-Aldrich) DMEM for 3×30 mins with agitation, dispase 5 mg/mL was added to the final 30 min digest step. A single cell suspension wash obtained upon washing with DMEM containing 10% FCS through a 70 μm strainer prior to analysis by flow cytometry.

### Flow Cytometry

For FACS surface staining the cells were labelled with the following anti-mouse antibodies (Biolegend sourced unless otherwise stated): CD3 (500A2, BUV495: 741064 BD Pharmingen), CD11b (M1/70, BUV395: 563553 BD Pharmingen), CD11c (N418, BV711: 117349), CD8 (53-6.7, BUV805: 612898 BD Pharmingen), CD4 (GK1.5, AF700: 100430), CD45 (2D1, FITC: 368507), CD11a (121/7, PECy7: 153108), CD69 (H1.2F3, BV650: 104541), Langerin (4C7, PE: 144204), Ly6C (RB6-8C5, BV605: 108440), Ly6G (1A8, PETxRed: 127648), MHCII (M5/114.15.2, BV785: 107645), SiglecF (S17007L, BV421: 155509), IL-17A (TC11-18H10.1, PECy7: 506922) Live/Dead Aqua (Invitrogen), and anti-human CD1a (APC or purified SK9, HI149, OKT6, NA1/34).

### Flow Cytometry: Epitope Competition Assay

CD1a-K562 cells were incubated with purified primary newly generated and commercially available anti-CD1a antibodies on ice for 30 minutes (25 μg/ml), the unbound antibody was then washed away and Alexa-Fluor-647 conjugated forms of the different antibodies were then incubated with the cells on ice for 30 minutes (10 μg/ml) in the matrix arrangement. Mean fluorescent intensity (MFI) was used to assess the degree of binding of the fluorophore conjugated antibody.

### Confocal Imaging

Murine ear skin was frozen in optimal cutting temperature embedding compound and stored at −80° C. 10 μm cryosections were cut using a Leica cryostat and collected onto Superfrost Plus slides to air-dry for 30 min before being stored at −80° C. Slides were rehydrated in PBS for 10 min before staining. The endogenous peroxidase activity of the sample was quenched by adding 0.15% hydrogen peroxide solution for 5 minutes at room temperature. Endogenous biotin was blocked with Avidin/Biotin Blocking Kit (Vector Laboratories Ltd), and 10% goat serum was used to reduce nonspecific binding of antibodies. Anti-CD1a antibody was used for confocal microscopy (1:100, OKT6; in-house production and conjugated to Biotin). Alexa Fluor 594 Tyramide SuperBoost kit (streptavidin; Thermo Fisher Scientific) was used to enhance the signal following manufacturer's instructions. Briefly, slides were incubated at 4° C. with primary antibodies overnight. After washing, HRP-conjugated streptavidin was added to the sections and incubated at 4° C. overnight. Excess streptavidin-HRP was washed away, the tissues were incubated with tyramide working solution for 8 min at room temperature, and the reaction was stopped with Reaction Stop Reagent. After staining, slides were mounted using antifade mounting medium with DAPI (Vector Laboratories Ltd), coverslips were applied, and slides were refrigerated in the dark until analyzed by confocal microscopy (Zeiss LSM 780 Confocal Microscope-Inverted Microscope; 25×/0.8 Imm Korr DIC M27; room temperature; Axiocam camera; Zen software), and Fiji was used for image processing.

### Cell Phenotype and Cytotoxicity Assays

Anti-CD1a antibodies and (5 μg/ml) commercially available comparator NA1/34 (5 μg/ml) were incubated with CD1a expressing K562 or EV control K562 for 48 hours and cell reduction assessed by flow cytometry. To measure direct antibody induced cell reduction, K562 were fluorescently labelled with CellTrace Violet prior to incubation with anti-CD1a antibodies for 48 hours. Prior to assessment of reduction by flow cytometry, a reference population of untreated CFSE labelled K562 was added to the antibody-treated K562 in a 1:1 ratio. The percentage of induced reduction was then calculated with the following equation by comparing the frequency of live cells of the different populations analysed, antibody treated and untreated reference CD1a+ and EV K562. % reduction=100−((% live cells of antibody-treated CD1a-K562/% live cells of reference CFSE labelled K562)/(% live cells of untreated CD1a-K562/% live cells of reference CFSE labelled K562)×100). To examine effects of anti-CD1a antibodies on apoptosis of CD1a-expressing cells, K562-CD1a or K562-EV were incubated with either isotype control or anti-CD1a antibodies (5 μg/ml) and stained for Annexin-V (Biolegend) 24 hours after incubation.

### Complement-Mediated Lysis (CDC) and Antibody-Dependent Cytotoxicity ADCC Assays

For CDC assays, K562-CD1a cells (5×10⁴ cells per well) were pre-treated with either 5 μg/ml isotype control antibody or indicated antibodies for 30 minutes and incubated with 10% normal human serum for 3-hours at 37° C. in 5% CO₂. For ADCC assays, fresh PBMCs were used. K562-CD1a cells (5×10³ cells per well) were co-cultured with PBMCs (2.5×10⁵ cells per well) for 5 h at 37° C. in 5% CO₂ with IL-2 (100 U/ml) in combination of either 5 μg/ml isotype control antibody or indicated antibodies (an effector/target ratio of 50:1). Cytotoxicity was determined by calculating the percentage of survived target K562-CD1a using the following equation: % cytotoxicity=((% live cells of CD1a-antibody-treated CD1a-K562/% live reference K562)/(% live cells of isotype-antibody-treated CD1a-K562/% live reference K562)×100).

### In Vivo CD1a+ Cell Depletion

“NSG” (NOD-scid IL2Rgamma^{null}) mice were subcutaneously injected with 0.25 million CD1a-K562 cells in ECM gel (Merck) suspension (vol=100 μl) to the flank and tumours were allowed to develop for 18 days. Mice were treated with 100 μg isotype control antibody or indicated antibodies on days 6, 10, and 14 intraperitoneally, and tumour size was measured.

### Isoelectric Focusing Assay (IEF)

Lipid loading was assessed by incubating 10 μg of CD1a with a 100× molar excess of imiquimod (Invivogen) solubilized in Tris Buffer saline and 2% CHAPS 7% DMSO or vehicle alone (mock) for 2 h at 37° C. and overnight at room temperature. CD1a samples were separated by isoelectric focusing (IEF). Briefly, CD1a-imiquimod and CD1a-mock proteins were loaded on an IEF pH 3-7 gel (Novex) that was then run for 1 hour at 100V, 1 hour and 200V and finally 30 mins at 500V. The gel was then fixed with 12% TCA and stained with SimplyBlue SafeStain for 7 minutes and destained in DI water overnight.

### Statistical Analysis

The one and two-way ANOVA tests were performed using GraphPad Prism version 6.00 (GraphPad Software). Error bars represent standard deviation as indicated.

### Generation and Selection of Therapeutic Anti-CD1a Antibodies 77A (VR11851), 110 (VR12112), 111 (VR12113), 116 (VR12117) and 16 (VR11834)

A number of animals across different species (including mice and rabbits) were immunized. Mice were immunized with NIH3T3 cells transfected with human CD1a and mouse B2M. Rabbits were immunized with Rab9 cells transfected with human CD1a and rabbit B2M. Following 3-5 shots, the animals were sacrificed and PBMC, spleen, bone marrow and lymph nodes harvested. Sera was monitored for binding to HEK-293 cells expressing human CD1a and human B2M via flow cytometry.

Memory B cell cultures (relevant for 77A (VR11851), 110 (VR12112), 111 (VR12113) and 116 (VR12117)) were set up and supernatants were first screened for their ability to bind HEK-293 cells transiently transfected with human CD1a in a bead-based assay on the TTP Labtech Mirrorball system. This was a multiplex assay using HEK-293 cells expressing human CD1a and human B2M stained with a cellular dye and counter-screened against counter-stained HEK-293 cells expressing CD1b, CD1c or CD1d with human B2M, using a goat anti-species Fc-FITC conjugate as a reveal agent.

Approx. 3500 CD1a-specific positive hits were identified in the primary Mirrorball screens from a total of 10×200-plate B culture experiments. Positive supernatants from this assay were then progressed for further characterization by:
ELISA, to confirm binding to human CD1a protein (details below)
ELISA, to confirm binding to the CD1a lipid binding domain on chimeric CD1a protein (human lipid binding domain of CD1a, mouse Ig domain of CD1d) (details below)
Flow cytometry, to confirm binding to human CD1a expressed on HEK-293 cells (co-expressed with human β2M) (details below)

Wells demonstrating binding in the above assays were progressed for V region recovery using the fluorescent foci method.

Plasma cells from bone marrow were also directly screened for their ability to bind human CD1a using the fluorescent foci method (relevant for 16 (VR11834)). Here, B cells secreting CD1a-specific antibodies were picked on biotinylated human CD1a immobilised on streptavidin beads using a goat anti-species Fc-FITC conjugate reveal reagent. Approx. 300 direct foci were picked.

Following reverse transcription (RT) and PCR of the picked cells, ‘transcriptionally active PCR’ (TAP) products encoding the antibodies' V regions were generated and used to transiently transfect HEK-293 cells. The resultant TAP supernatants, containing recombinant antibody, were further characterized by:
ELISA, to confirm binding to human CD1a protein and chimeric CD1a protein (human lipid binding domain of CD1a, mouse Ig domain of CD1d) (details below)
Flow cytometry, to confirm binding to human CD1a expressed on HEK-293 cells (co-expressed with human β2M) and counter-screen for cross-binding to relevant similar proteins: CD1b, CD1c or CD1d expressed on HEK-293 cells (co-expressed with human β2M). (details below)

Heavy and light chain variable region gene pairs from interesting TAP products were then cloned as either rabbit or mouse full length antibodies and re-expressed in a HEK-293 transient expression system. In total 119 V regions were cloned and registered. Recombinant cloned antibodies were then further characterized by:
Repeats of the above flow cytometry and ELISA assays.
Flow cytometry, to assess binding to CD1a expressed in multiple cell lines. This gave an initial indication that binding was lipid independent. Supernatants were screened for binding to:
   Stably transduced C1R cells expressing CD1a or empty vector (co-expressed with human β2M). These are relevant for 110 (VR12112), 111 (VR12113) and 116 (VR12117). (details below)
   MOLT4 cells endogenously expressing CD1a, CD1b, CD1c, CD1d and β2M. These are relevant for 110 (VR12112), 111 (VR12113) and 116 (VR12117). (details below)
Profiling in BIAcore to estimate off-rate and affinity (details below)

Antibodies demonstrating binding in the above assays and <100 nM affinity were selected for purification. Cell culture supernatants were purified using Protein A affinity purification. Purified samples were buffer exchanged in to 10 mM PBS pH 7.4 and analysed for its recovery and purity using UV spectroscopy, analytical size exclusion chromatography, SDS Page electrophoresis and LAL endotoxin assay respectively. Where required samples were subject to second round of purification to increase the monomer levels. Final samples were sterile filtered and stored in 10 mM PBS pH 7.4

Following purification, all 5 antibodies were then further characterized by:
Repeats of the above flow cytometry, ELISA and BIAcore assays
ELISA, to assess binding to Cynomolgus monkey CD1a protein and the variant of human CD1a protein common in China (18) (details below)
Flow cytometry, to assess binding to HEK-293 cells transiently transfected with: (details below)
   Cynomolgus monkey CD1a co-transfected with Cynomolgus monkey β2M
   The variant of human CD1a common in China co-transfected with human β2M

77A (VR11851), 110 (VR12112), 111 (VR12113), 116 (VR12117) and 16 (VR11834) demonstrated the capacity to bind to all tested forms of recombinant and cell expressed CD1a proteins at the respective stages of antibody discovery (Tables 1-9). The only exception was 116 (VR12117) which showed no binding to recombinant or cell expressed Cynomolgus CD1a (Table 4 and 9). Inclusion of antibody 116 in the subsequent in vitro and in vivo analyses was not considered obvious but was nevertheless a deliberate step in order to focus on epitope binding regions where the lipid-binding domain differs from human and cynomolgus with potentially different functional effects. None of the antibodies demonstrated binding to CD1b, CD1c or CD1d expressed on HEK-293 cells (Table 5), indicating these antibodies are CD1a-specific. CD1a, CD1b, CD1c and CD1d expression in HEK-293 cells was confirmed with commercially available antibodies, supporting this conclusion (data not shown). Binding to CD1a expressed on multiple cell types (HEK, C1R and MOLT4) gave an initial indication that antibody binding may be lipid-independent as CD1a is likely loaded from a different pool of lipids in each cell line.

Following antibody discovery, the antibodies were assessed for in vitro function in T cell assays as below.

DNA encoding the heavy and light chain V-regions of 77A (VR11851), 110 (VR12112), 111 (VR12113) and 116 (VR12117) on a mouse IgG1 backbone was synthesized at ATUM and expressed in a HEK-293 transient expression system in house. The antibodies then underwent purification and endotoxin removal and were tested in in vivo assays, as below.

### Affinity of 77A (VR11851), 110 (VR12112), 111 (VR12113), 116 (VR12117) and 16 (VR11834) for Human CD1a

The affinity of the purified antibodies to human CD1a was assessed using a Biacore T200 instrument (GE Healthcare) by capturing the antibody to an immobilized anti-species IgG F(ab′)2 followed by titration of human CD1a. Affinipure Goat anti-species IgG-F(ab′)2 fragment specific (Jackson ImmunoResearch) was immobilized on a CM5 Sensor Chip (GE Healthcare) via amine coupling chemistry to a capture level of ˜5000 response units (RUs). HBS-EP+ buffer (10 mM HEPES pH 7.4, 0.15 M NaCl, 3 mM EDTA, 0.05% Surfactant P20, GE Healthcare) was used as the running buffer with a flow rate of 10 μL/min. A 10 μL injection of test antibody at 0.5 μg/mL was used for capture by the immobilized Goat Anti-species Fab. Human CD1a was titrated over the captured antibodies (at 0 nM, 0.6 nM, 1.8 nM, 5.5 nM, 16.6 nM and 50 nM, diluted in running buffer) at a flow rate of 30 μL/min to assess affinity.

The surface was regenerated between cycles by injection of 2×10 μL of 40 mM HCl, interspersed by a 10 μL injection of 5 mM NaOH at flowrate of 10 μL/min. Background subtraction binding curves were analyzed using the Biacore T200 evaluation software following standard procedures. Kinetic parameters were determined from the fitting algorithm. This assay was performed at the clone supernatant and purified antibody stage. The kinetic parameters of antibody binding to human CD1a are shown in Table 10.

### Binding of 77A (VR11851), 110 (VR12112), 111 (VR12113), 116 (VR12117) and 16 (VR11834) Assessed by ELISA

CD1a-specific antibodies were identified by ELISA. ELISA plates were coated with 2 μg/mL protein of interest (human CD1a pool B, chimeric CD1a pool B [human lipid binding domain and mouse CD1d Ig domain], Chinese variant CD1a or Cynomolgus CD1a) (20 μL/well) at 4° C. overnight and then washed with wash buffer (0.2% (v/v) Tween-20 in PBS (pH7.4). Plates were then blocked with 80 μl/well block buffer (1% (w/v) bovine serum albumin) for 1 hour at room temperature and then washed in wash buffer. 20 μL antibody sample (B cell culture supernatant, TAP supernatant, clone supernatant, purified antibody solution) dilutions was transferred to the ELISA plates and incubated at room temperature for 1 hour, followed by washing with wash buffer. 20 μl/well of peroxidase-conjugated goat anti-species IgG Fc-specific F(ab′)2 fragment (Jackson ImmunoResearch), diluted 1:5000 in block buffer was added and incubated at room temperature for 1 hour, followed by washing with wash buffer. TMB substrate (EMD Millipore) was added (20 μL/well) to visualize binding, and the reaction incubated at room temperature for 5 minutes before measuring the optical density at 630 nM using a microplate reader. This assay was performed at the B-cell supernatant stage (human CD1a pool B), TAP supernatant stage (human CD1a pool B, chimeric CD1a pool B), clone supernatant stage (human CD1a pool B, chimeric CD1a pool B) and purified antibody stage (human CD1a pool B, chimeric CD1a pool B, Chinese variant CD1a, Cynomolgus CD1a). Data for purified antibodies shown in Tables 1-4.

### Binding of 77A (VR11851), 110 (VR12112), 111 (VR12113), 116 (VR12117) and 16 (VR11834) Assessed by Flow Cytometry

CD1a-specific antibodies were identified by flow cytometry. Binding to proteins expressed on HEK, C1R and MOLT4 cell lines was assessed. HEK-293 cells were transfected with a protein of interest (CD1a, CD1b, CD1c, CD1d, Chinese variant CD1a or Cynomolgus CD1a) and the species-specific β2M (as indicated above). The transfections were performed using the Expifectamine 293 kit (Gibco) and incubated overnight. The C1R-CD1a, C1R-empty vector and MOLT4 cell lines were washed in 1×PBS on the day required. All cell lines were counted and resuspended in 1×PBS and then stained for 30 minutes at 37° C. using the DiI or DiO cellular stains (Invitrogen). Cells were washed with flow cytometry buffer (1% bovine serum albumin, 2 mM EDTA and 0.1% sodium azide in PBS) before mixing 2 DiI-stained and DiO-stained populations together. The cells (20 μl/well) were then added to dilutions of antibody sample (B cell culture supernatant, TAP supernatant, clone supernatant, purified antibody solution) (20 μl/well) and incubated for 1 hour at 4° C. in a flow cytometry assay plate, before being washed with flow cytometry buffer. 10 μl/well of Alexafluor647-conjugated goat anti-species IgG Fc-specific F(ab′)2 fragment (Jackson ImmunoResearch), diluted 1:2500 in flow cytometry buffer, was added and incubated at 4° C. for 30 minutes, followed by washing with wash buffer. The fluorescence intensity was then measured on an iQue screener PLUS. This assay was performed at the B-cell supernatant stage (HEK-293 cells expressing human CD1a), TAP supernatant stage (HEK-293 cells expressing human CD1a, CD1b, CD1c or CD1d), clone supernatant stage (HEK-293 cells expressing human CD1a, CD1b, CD1c or CD1d; C1R cells expressing human CD1a or empty vector; MOLT4 cell line) and purified antibody stage (HEK-293 cells expressing human CD1a, CD1b, CD1c, CD1d, Chinese variant CD1a or Cynomolgus CD1a; C1R cells expressing human CD1a or empty vector; MOLT4 cells). Data for purified antibodies is shown in Tables 5-9.

TABLE 1
Antibody binding to human CD1a pool B protein. 77A (VR11851), 110
(VR12112), 111 (VR12113), 116 (VR12117) and 16 (VR11834) were
tested for their ability to bind human CD1a protein in an ELISA.
The antibodies were titrated through a dilution series and compared
to a control rabbit IgG antibody. All 5 antibodies bound to human
CD1a pool B protein. Data shown for purified antibodies.

| Antibody | Optical Density (OD) | | | | | | | |
|---|---|---|---|---|---|---|---|---|
| Concen- | 100 | 31.6 | 10 | 3.16 | 1 | 0.316 | 0.1 | 0.0316 |
| tration | μg/ml | μg/ml | μg/ml | μg/ml | μg/ml | μg/ml | μg/ml | μg/ml |
| 77A | 1.39 | 1.28 | 1.34 | 1.33 | 1.34 | 1.39 | 1.36 | 1.25 |
| (VR11851) | | | | | | | | |
| 110 | 1.23 | 1.24 | 1.28 | 1.27 | 1.19 | 1.18 | 1.12 | 0.81 |
| (VR12112) | | | | | | | | |
| 111 | 1.12 | 1.07 | 1.12 | 1.13 | 1.14 | 1.04 | 1.06 | 1.10 |
| (VR12113) | | | | | | | | |
| 116 | 1.15 | 1.16 | 1.18 | 1.15 | 1.15 | 1.15 | 1.07 | 0.95 |
| (VR12117) | | | | | | | | |
| 16 | NA | NA | NA | NA | NA | NA | 0.59 | NA |
| (VR11834) | | | | | | | | |
| Control IgG | 0.07 | 0.06 | 0.07 | 0.06 | 0.07 | 0.07 | 0.06 | 0.06 |

TABLE 2
Antibody binding to chimeric CD1a pool B protein. 77A (VR11851),
110 (VR12112), 111 (VR12113), 116 (VR12117) and 16 (VR11834)
were tested for their ability to bind chimeric CD1a [human
CD1a lipid binding domain, mouse CD1d Ig domain] protein
in an ELISA. The antibodies were titrated through a dilution
series and compared to a control rabbit IgG antibody. All
5 antibodies bound to chimeric CD1a pool B protein. Data
shown for purified antibodies.

| Antibody | Optical Density (OD) | | | | | | | |
|---|---|---|---|---|---|---|---|---|
| Conc. | 100 | 31.6 | 10 | 3.16 | 1 | 0.316 | 0.1 | 0.0316 |
| (μg/ml | μg/ml | μg/ml | μg/ml | μg/ml | μg/ml | μg/ml | μg/ml | μg/ml |
| 77A | 1.27 | 1.33 | 1.31 | 1.28 | 1.26 | 1.28 | 1.25 | 1.09 |
| (VR11851) | | | | | | | | |
| 110 | 1.40 | 1.40 | 1.46 | 1.53 | 1.45 | 1.24 | 1.02 | 0.73 |
| (VR12112) | | | | | | | | |
| 111 | 1.27 | 1.35 | 1.40 | 1.41 | 1.36 | 1.32 | 1.25 | 1.19 |
| (VR12113) | | | | | | | | |
| 116 | 1.37 | 1.35 | 1.40 | 1.38 | 1.42 | 1.32 | 1.25 | 1.13 |
| (VR12117) | | | | | | | | |
| 16 | NA | NA | NA | NA | NA | NA | 0.70 | NA |
| (VR11834) | | | | | | | | |
| Control IgG | 0.08 | 0.08 | 0.08 | 0.10 | 0.09 | 0.09 | 0.08 | 0.09 |

TABLE 3
Antibody binding to Chinese variant CD1a protein. 77A (VR11851),
110 (VR12112), 111 (VR12113), 116 (VR12117) and 16 (VR11834)
were tested for their ability to bind Chinese variant CD1a
protein in an ELISA. The antibodies were titrated through
a dilution series and compared to a control rabbit IgG
antibody. All 5 antibodies bound to Chinese variant CD1a
protein. Data shown for purified antibodies.

| | Optical Density (OD) | | | |
|---|---|---|---|---|
| Antibody | 10 | 1 | 0.1 | 0.01 |
| Concentration | μg/ml | μg/ml | μg/ml | μg/ml |
| 77A | 0.99 | 0.78 | 1.05 | 0.79 |
| (VR11851) | | | | |
| 110 | 1.75 | 1.79 | 1.57 | 1.40 |
| (VR12112) | | | | |
| 111 | 1.41 | 1.44 | 1.52 | 1.33 |
| (VR12113) | | | | |
| 116 | 1.51 | 1.51 | 1.53 | 1.47 |
| (VR12117) | | | | |
| 16 | 1.44 | 1.35 | 1.30 | 1.01 |
| (VR11834) | | | | |
| Control IgG | 0.14 | 0.08 | 0.08 | 0.08 |

TABLE 4
Antibody binding to Cynomolgus monkey CD1a protein. 77A
(VR11851), 110 (VR12112), 111 (VR12113), 116 (VR12117)
and 16 (VR11834) were tested for their ability to bind
Cynomolgus CD1a protein in an ELISA. The antibodies
were titrated through a dilution series and compared
to a control rabbit IgG antibody. All 5 antibodies,
except 116 (VR12117), bound to Cynomolgus monkey CD1a
protein. Data shown for purified antibodies.

Optical Density (OD)

| Antibody | 10 | 1 | 0.1 | 0.01 |
|---|---|---|---|---|
| Concentration | μg/ml | μg/ml | μg/ml | μg/ml |
| 77A | 0.52 | 0.43 | 0.24 | 0.10 |
| (VR11851) | | | | |
| 110 | 0.86 | 0.94 | 0.53 | 0.22 |
| (VR12112) | | | | |
| 111 | 0.79 | 0.69 | 0.66 | 0.48 |
| (VR12113) | | | | |
| 116 | 0.16 | 0.14 | 0.07 | 0.07 |
| (VR12117) | | | | |
| 16 | 0.49 | 0.58 | 0.57 | 0.46 |
| (VR11834) | | | | |
| Control IgG | 0.09 | 0.07 | 0.07 | 0.06 |

TABLE 5
Antibody binding to human CD1a, CD1b, CD1c or CD1d expressed on HEK-293
cells. HEK-293 cells were transiently transfected with human CD1a, CD1b,
CD1c or CD1d and co-transfected with human β2M. 77A (VR11851), 110 (VR12112),
111 (VR12113), 116 (VR12117) and 16 (VR11834) were titrated through a dilution
series and tested for binding to the transfected proteins. Binding was
quantified as fold change in fluorescence intensity geomean over background
assessed by flow cytometry. All 5 antibodies bound to human CD1a expressed
on HEK-293 cells. No binding to CD1b, CD1c or CD1d expressed on HEK-293
cells was observed. Data shown for purified antibodies.

| | | Fluorescence Intensity Geomean | | | | | | | |
|---|---|---|---|---|---|---|---|---|---|
| | | (normalized to background) | | | | | | | |
| Targe | Antibody | 100 | 31.6 | 10 | 3.16 | 1 | 0.316 | 0.1 | 0.0316 |
| t cell | Concentration | μg/ml | μg/ml | μg/ml | μg/ml | μg/ml | μg/ml | μg/ml | μg/ml |
| HEK- | 77A (VR11851) | 4.1 | 8.2 | 13.7 | 23.3 | 41.9 | 58.6 | 43.8 | 24.9 |
| CD1a | 110 (VR12112) | 5.9 | 10.9 | 15.7 | 31.5 | 49.2 | 38.3 | 20.2 | 8.9 |
| | 111 (VR12113) | 4.3 | 6.3 | 11.1 | 24.9 | 42.0 | 30.8 | 23.1 | 11.1 |
| | 116 (VR12117) | 3.9 | 5.6 | 12.6 | 21.9 | 48.2 | 36.5 | 26.7 | 15.7 |
| | 16 (VR11834) | NA | NA | 10.2 | NA | 19.6 | NA | 12.9 | NA |
| HEK- | 77A (VR11851) | 1.0 | 1.0 | 1.1 | 0.9 | 1.1 | 1.1 | 1.0 | 0.9 |
| CD1b | 110 (VR12112) | 1.1 | 1.1 | 1.1 | 1.1 | 1.1 | 1.0 | 1.0 | 1.0 |
| | 111 (VR12113) | 1.1 | 1.0 | 1.1 | 1.1 | 1.0 | 1.0 | 1.0 | 1.0 |
| | 116 (VR12117) | 1.1 | 1.0 | 1.0 | 1.0 | 1.1 | 1.0 | 1.0 | 1.0 |
| | 16 (VR11834) | NA | NA | NA | NA | NA | NA | NA | NA |
| HEK- | 77A (VR11851) | 0.9 | 0.9 | 0.9 | 0.9 | 1.0 | 1.0 | 0.9 | 1.0 |
| CD1c | 110 (VR12112) | 1.0 | 1.0 | 1.0 | 1.1 | 1.1 | 0.9 | 1.0 | 1.0 |
| | 111 (VR12113) | 1.1 | 1.0 | 1.0 | 1.1 | 1.1 | 1.0 | 1.0 | 1.0 |
| | 116 (VR12117) | 1.0 | 1.0 | 1.1 | 1.1 | 1.1 | 1.1 | 1.0 | 1.0 |
| | 16 (VR11834) | NA | NA | NA | NA | NA | NA | NA | NA |
| HEK- | 77A (VR11851) | 1.0 | 1.0 | 1.0 | 0.9 | 1.0 | 1.0 | 0.9 | 0.9 |
| CD1d | 110 (VR12112) | 1.1 | 1.1 | 1.0 | 1.0 | 1.1 | 1.0 | 1.0 | 1.0 |
| | 111 (VR12113) | 1.1 | 1.0 | 1.1 | 1.1 | 1.1 | 0.9 | 1.0 | 1.0 |
| | 116 (VR12117) | 1.0 | 1.0 | 1.1 | 1.1 | 1.0 | 1.0 | 1.1 | 1.0 |
| | 16 (VR11834) | NA | NA | NA | NA | NA | NA | NA | NA |

TABLE 6
Antibody binding to human CD1a, CD1b, CD1c or CD1d expressed on C1R cells.
77A (VR11851), 110 (VR12112), 111 (VR12113), 116 (VR12117) and 16 (VR11834)
were titrated through a dilution series and tested for binding to C1R
cells stably transduced with human CD1a or empty vector and human β2M.
Binding was quantified as fold change in fluorescence intensity geomean
over background assessed by flow cytometry. All 5 antibodies bound to
human CD1a expressed on C1R cells. No binding to C1R cells expressing
empty vector was observed. Data shown for purified antibodies.

| | | Fluorescence Intensity Geomean | | | | | | | |
|---|---|---|---|---|---|---|---|---|---|
| | | (normalized to background) | | | | | | | |
| Targe | Antibody | 100 | 31.6 | 10 | 3.16 | 1 | 0.316 | 0.1 | 0.0316 |
| t cell | Concentration | μg/ml | μg/ml | μg/ml | μg/ml | μg/ml | μg/ml | μg/ml | μg/ml |
| CIR - | 77A (VR11851) | 0.9 | 1.3 | 2.3 | 4.2 | 10.3 | 19.0 | 16.4 | 17.0 |
| CD1a | 110 (VR12112) | 4.8 | 6.2 | 8.3 | 15.2 | 28.2 | 29.7 | 25.8 | 17.1 |
| | 111 (VR12113) | 7.3 | 7.0 | 11.7 | 15.6 | 28.0 | 28.8 | 25.4 | 26.0 |
| | 116 (VR12117) | 11.9 | 13.7 | 16.9 | 24.0 | 28.7 | 27.5 | 27.0 | 24.1 |
| | 16 (VR11834) | NA | NA | 7.0 | NA | 10.1 | NA | 10.2 | NA |
| C1R - | 77A (VR11851) | 1.0 | 1.0 | 1.0 | 1.0 | 1.0 | 1.0 | 1.0 | 0.9 |
| Empty | 110 (VR12112) | 1.0 | 0.8 | 0.9 | 1.0 | 0.9 | 0.8 | 0.9 | 0.4 |
| Vector | 111 (VR12113) | 1.1 | 1.0 | 1.0 | 1.0 | 0.9 | 0.9 | 1.0 | 1.2 |
| | 116 (VR12117) | 1.0 | 1.0 | 1.2 | 1.4 | 1.3 | 1.0 | 1.0 | 0.9 |
| | 16 (VR11834) | NA | NA | 0.9 | NA | 0.9 | NA | 0.9 | NA |

TABLE 7
Antibody binding to MOLT4 cells. 77A (VR11851), 110 (VR12112),
111 (VR12113), 116 (VR12117) and 16 (VR11834) were titrated
through a dilution series and tested for binding to MOLT4
cells which endogenously express CD1a, CD1b, CD1c, CD1d and
β2M. Binding was quantified as fold change in fluorescence
intensity geomean over background assessed by flow cytometry.
All 5 antibodies bound to MOLT4 cell surface proteins, most
likely CD1a. Data shown for purified antibodies.

| | Fluorescence Intensity Geomean | | | | | | | |
|---|---|---|---|---|---|---|---|---|
| Antibody | (normalized to background) | | | | | | | |
| Concen- | 100 | 31.6 | 10 | 3.16 | 1 | 0.316 | 0.1 | 0.0316 |
| tration | μg/ml | μg/ml | μg/ml | μg/ml | μg/ml | μg/ml | μg/ml | μg/ml |
| 77A | 0.8 | 2.2 | 4.1 | 7.4 | 13.5 | 15.2 | 12.4 | 8.7 |
| (VR11851) | | | | | | | | |
| 110 | 5.2 | 7.5 | 14.7 | 22.6 | 33.0 | 27.1 | 16.8 | 1.6 |
| (VR12112) | | | | | | | | |
| 111 | 3.9 | 5.4 | 9.3 | 18.9 | 31.2 | 27.3 | 20.6 | 12.4 |
| (VR12113) | | | | | | | | |
| 116 | 3.7 | 5.6 | 11.7 | 23.9 | 30.6 | 29.6 | 22.6 | 14.9 |
| (VR12117) | | | | | | | | |
| 16 | NA | NA | 7.5 | NA | 15.0 | NA | 14.4 | NA |
| (VR11834) | | | | | | | | |

TABLE 8
Antibody binding to a common Chinese variant CD1a expressed
on HEK-293 cells. 77A (VR11851), 110 (VR12112), 111 (VR12113),
116 (VR12117) and 16 (VR11834) were titrated through a dilution
series and tested for binding to HEK-293 cells transiently
transfected with a common Chinese variant CD1a (18) and human
β2M. Binding was quantified as fold change in fluorescence
intensity geomean over background assessed by flow cytometry.
All 5 antibodies bound to Chinese variant CD1a expressed on
HEK-293 cells. Data shown for purified antibodies.

Fluorescence Intensity Geomean
(normalized to background)

| Antibody | 10 | 1 | 0.1 | 0.01 |
|---|---|---|---|---|
| Concentration | μg/ml | μg/ml | μg/ml | μg/ml |
| 77A | 6.9 | 47.2 | 99.3 | 76.4 |
| (VR11851) | | | | |
| 110 | 48.3 | 111.5 | 119.1 | 29.1 |
| (VR12112) | | | | |
| 111 | 35.4 | 39.5 | 96.4 | 33.8 |
| (VR12113) | | | | |
| 116 | 48.9 | 12.8 | 100.0 | 34.5 |
| (VR12117) | | | | |
| 16 | 11.4 | 11.2 | 11.2 | 6.0 |
| (VR11834) | | | | |

TABLE 9
Antibody binding to Cynomolgus monkey CD1a expressed on HEK-293
cells. 77A (VR11851), 110 (VR12112), 111 (VR12113), 116 (VR12117)
and 16 (VR11834) were titrated through a dilution series and
tested for binding to HEK-293 cells transiently transfected
with Cynomolgus monkey CD1a and Cynomolgus monkey β2M. Binding
was quantified as fold change in fluorescence intensity geomean
over background assessed by flow cytometry. All 5 antibodies,
except 116 (VR12117), bound to Cynomolgus monkey CD1a expressed
on HEK-293 cells. Data shown for purified antibodies.

Fluorescence Intensity Geomean
(normalized to background)

| Antibody | 10 | 1 | 0.1 | 0.01 |
|---|---|---|---|---|
| Concentration | μg/ml | μg/ml | μg/ml | μg/ml |
| 77A | 1.4 | 10.6 | 5.7 | 1.7 |
| (VR11851) | | | | |
| 110 | 13.6 | 11.2 | 3.3 | 1.2 |
| (VR12112) | | | | |
| 111 | 16.3 | 12.9 | 25.8 | 8.9 |
| (VR12113) | | | | |
| 116 | 1.7 | 0.8 | 1.0 | 1.0 |
| (VR12117) | | | | |
| 16 | 8.8 | 7.6 | 8.5 | 6.9 |
| (VR11834) | | | | |

TABLE 10
Antibody affinity for human CD1a. The affinity of 77A (VR11851), 110
(VR12112), 111 (VR12113), 116 (VR12117) and 16 (VR11834) for human
CD1a was assessed using biacore. The 1:1 binding model was used to
fit the data in all cases, except 16 (VR11834) which required the heterogenous
ligand binding model. Affinity was required to be <100 nM to be
considered for progression. Data shown for purified antibodies.

| | ka | ka 2 | kd | kd 2 | KD |
|---|---|---|---|---|---|
| Ligand | (1/Ms) | (1/Ms) | (1/s) | (1/s) | (M) |
| 77A | 4.69E+05 | | 1.13E−04 | | 2.40E−10 |
| (VR11851) | | | | | |
| 110 | 4.43E+05 | | 8.74E−04 | | 1.97E−09 |
| (VR12112) | | | | | |
| 111 | 3.21E+05 | | 1.47E−08 | | 3.12E−11 |
| (VR12113) | | | | | |
| 116 | 4.83E+05 | | 1.40E−03 | | 2.89E−09 |
| (VR12117) | | | | | |
| 16 | 1.86E+06 | 5.14E+05 | 7.29E−04 | 6.58E−03 | 3.92E−10 |
| (VR11834) | | | | | |

| | KD2 | Chi² | Ligand | | Rmax |
|---|---|---|---|---|---|
| Ligand | (M) | (RU²) | Level (RU) | Model | (RU) |
| 77A | | 0.306 | 177.2 | 1:1 Binding | 118.5 |
| (VR11851) | | | | | |
| 110 | | 3.94E−02 | 128.1 | 1:1 Binding | 24.2 |
| (VR12112) | | | | | |
| 111 | | 2.48E−02 | 68.5 | 1:1 Binding | Local Fit |
| (VR12113) | | | | | |
| 116 | | 1.19E−01 | 120.3 | 1:1 Binding | 49.0 |
| (VR12117) | | | | | |
| 16 | 1.28E−08 | NA | 78.1 | Heterogeneous | NA |
| (VR11834) | | | | Ligand | |

### EXAMPLES

### Example 1—Anti-CD1a Panel Refinement: Functional Assessment of Anti-CD1a Antibodies

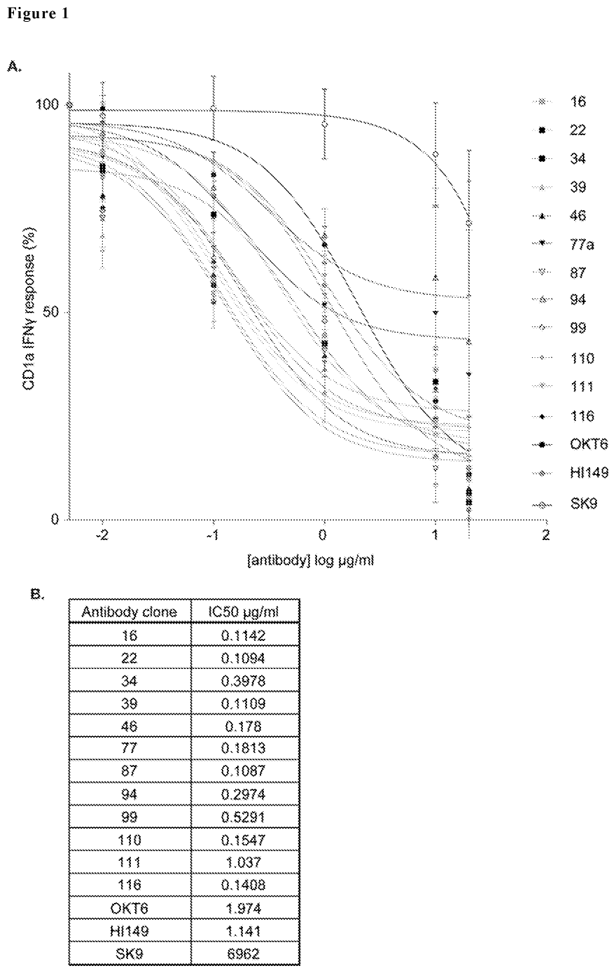

Following CD1a binding assessment a large panel of anti-CD1a antibodies generated for inhibitory function were screened. T cell cytokine production was measured in an in vitro antigen presentation model by EliSpot. A summary of these data is presented in FIG. 1. It was determined that a number of the newly generated antibodies were more potent in the inhibition of CD1a T cell responses than commercial anti-CD1a antibodies OKT6, HI149 and SK9. Of note, antibodies 16, 22, 39, 46, 77, 87, 110, 116 all had at least a log lower IC50 than OKT6 (FIG. 1B) which is an improvement over antibodies described in the prior art, despite the use of polyclonal T cells which would be expected to be less sensitive than transduced clonal immortal T-cells.

### Example 2—Anti-CD1a Panel Refinement: Inhibition of CD1a-Restricted Enriched T Cell Lines Responses

To aid the short listing of antibody candidates for in vivo analyses, a different approach was taken to assess CD1a T cell responses; CD1a-restricted enriched T cell lines were isolated and expanded to analyse the CD1a response in isolation, rather than in a mixed polyclonal T cell background where the low signal to noise ratio can partially mask the potential of the inhibitory antibodies.

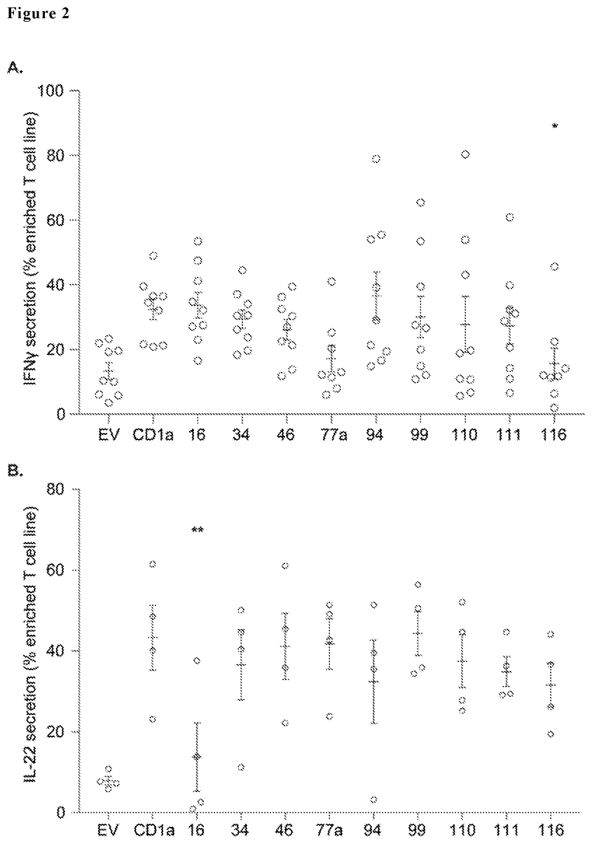

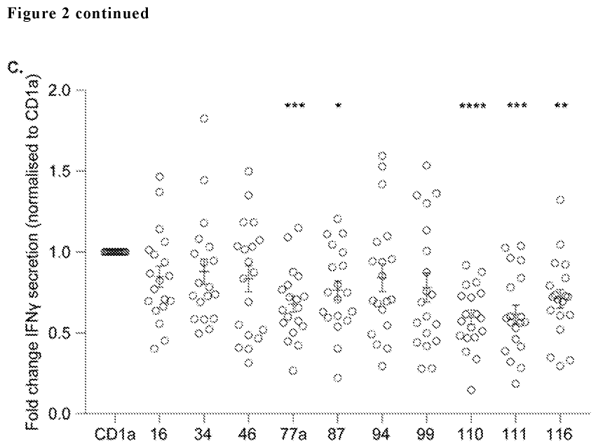

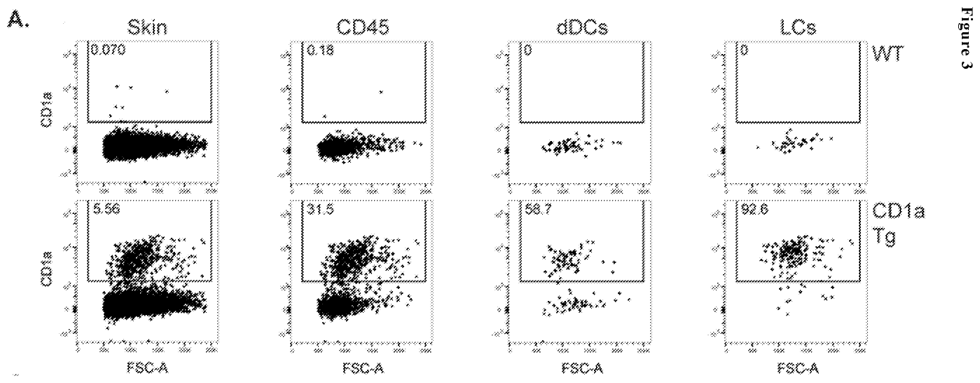

In these assays antibodies 116 and 16 stood out as potent inhibitory antibodies, with 16 uniquely inhibiting the autoreactive/endogenous production of IL-22 (FIGS. 2A and 2B). This improvement shows the possibility of using the antibodies in conditions on which IL-22 plays a pathogenic role, in addition to conditions which have a role for IFNγ. It was surprising to see differential effects on different cytokines. Further, an APC-free system was used to assess antibody dependent inhibition of CD1a-restricted T cell activation. CD1a-coated beads were used as a surrogate for the APC, and resulting T cell IFNγ production was measured by flow cytometry. This assay revealed significant inhibition of the CD1a-dependent cytokine response with all antibodies, but particularly 77a, 87, 110, 111 and 116 (FIG. 2C).

### Example 3—In Vivo Assessment of Inhibitory Antibodies in Skin Inflammation

The aim of this study has been to produce antibodies that would be of clinical use in treating human diseases and disorders, thus it was essential to ascertain efficacy in a complex immune system akin to human disease. A highly refined panel of the best of the newly generated antibodies were chosen from analysis of the above data (antibodies 16, 77a, 110, 111 and 116), and it was sought to determine their potential in an in vivo model of psoriasis, dermatitis, lupus and as a model of drug reactions which manifest as an inflammatory skin or mucosal disease or disorder, or associated systemic disease or disorder, or one or more inflammatory drug reaction which manifests systemically. Experimental psoriasis and dermatitis have been shown to be exacerbated in the CD1a transgenic mouse as compared to WT, and the CD1a-dependent inflammation can be ameliorated with administration of anti-CD1a antibody (Kim et al 2016). It is also of note that some individuals develop a skin/mucosal inflammatory drug reaction to imiquimod, used topically for a number of skin disorders; such drug reactions include psoriatic reactions, dermatitis reactions, bullous disease, alopecia, vesiculation, lichenoid reactions, neutrophilic diseases, lupus erythematosus, erythema multiforme, oral erosions and severe drug reactions such as DRESS, AGEP, Stevens-Johnson syndrome and toxic epidermal necrolysis (19-29).

### Generation of CD1a Transgenic Mice

To assess a possible role for CD1a in skin and associated systemic inflammation the inventors generated a CD1a transgenic mouse. CD1a is absent from the mouse genome, and so the human CD1a gene locus with 0.8 kb 5′ and 0.8 kb 3′ flanking region that includes the promoter element, was cloned and the transgene inserted by microinjection, akin to the published CD1a transgenic model, but requiring additional transgene fragment stitching (Illing et al., Nature 486, 554-558 (2012)). The genotype positive founder mice were bred and lines screened for CD1a transgene expression. The inventors went on to phenotype the mice and determine whether CD1a protein expression followed the expected profile and was representative of human CD1a cellular expression. Ear skin of wild-type and CD1a transgenic (CD1aTg) mice was collected and enzymatically processed to allow analysis of the cutaneous cellular environment by flow cytometry (FIG. 3A). CD1a expression was detected in the skin constituting 4.2% (+/−1.79) of total skin cells and 23.6% (+/−6.68) of CD45+ cells. To assess the cellular regulation of expression, dermal DCs (dDCs) and Langerhans cells (LCs) were assessed for CD1a protein. Dermal DC subsets have been reported to express CD1a and Langerhans cells are characteristically constitutive CD1a^{high}. CD1a was found to be expressed by 41.5% (+/−20.38) of dDCs and 88% (+/−4.606) of LCs (FIG. 3A-B). CD1a protein expression was further characterised in the skin by immunofluorescence revealing characteristic epidermal location and cells with dendrites typical of LCs (FIG. 3C). CD1a genotype was confirmed (FIG. 3D), and CD1a expression within the thymus was observed, predominantly by a proportion of CD4+CD8+ double positive thymocytes (FIG. 3E). CD1aTg mice showed no aberrant skin inflammation at steady state. In summary, the inventors generated a CD1a transgenic mouse that displays CD1a expression in a manner phenotypically analogous to human tissue expression.

This model was used to test the anti-CD1a antibodies for prevention of inflammatory skin diseases and disorders (FIG. 4A). Application of Aldara cream, containing 5% imiquimod a TLR7/8 agonist, is an established model which induces psoriasis-like, dermatitis-like, lupus-like skin inflammation typified by skin thickening, scaling and reddening (30, 31). It was found that inflammation of the ear of CD1a-transgenic mice was considerably higher than of WT counterparts in response to Aldara. Furthermore, all anti-CD1a antibodies administered before the imiquimod reduced subsequent ear thickening, however antibodies 116 and 16 ablated CD1a-dependent inflammation to at least the WT level (FIG. 4B). By the end point of the experiment CD1a-transgenic (−Tg) mice treated with antibodies 16 and 110 showed reduction of inflammation to the WT level of ear thickening. Strikingly and unexpectedly, antibody 116 treatment reduced the level of CD1a-Tg ear skin inflammation significantly below that of WT skin (FIG. 4B).

Example 4—In vivo effects of inhibitory antibodies on the skin immune response It was sought to analyse the contribution of cutaneous immune populations to imiquimod-induced CD1a-dependent ear inflammation.

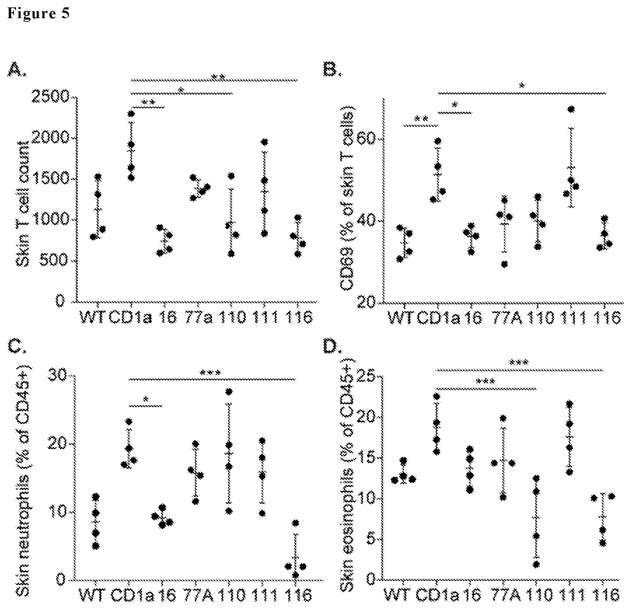

It was found that skin T cell infiltration was elevated in the CD1a transgenic mouse and the frequency of this population was reduced by the anti-CD1a antibodies, in particular antibodies 116, 16 and 110 in the prevention model (FIG. 5A). Of note, 16 and 116 were able to reduce skin T cell infiltrate to levels below wild-type suggesting an improved and profound effect on inflammation in vivo. Furthermore, activation marker CD69 was increased on the surface of skin T cells in the CD1a transgenic mouse, and was inhibited by some of the anti-CD1a antibodies, in particular 116 and 16 in the prevention model (FIG. 5B). Neutrophils are known to be important cells of a number of inflammatory disorders, including the psoriatic response and the murine imiquimod model. Here, elevated neutrophil frequency was found in the skin upon imiquimod treatment and further increase in the CD1a transgenic mouse, which was reduced to the WT level or below with anti-CD1a antibodies 116 and 16 in the prevention model (FIG. 5C). A reduction in skin eosinophils in response to the antibodies was also noted, which is of interest given the known role of eosinophils in many forms of drug reactivity (FIG. 5D). This unexpected finding represents an improvement as effects on eosinophils have not previously been observed.

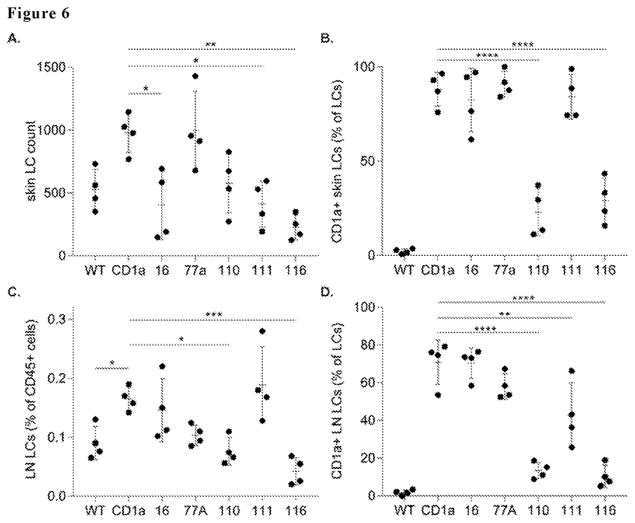

Langerhans cells, defined here as CD11c+ Langerin+, were also increased, compared to WT, in the skin upon imiquimod challenge of the CD1a transgenic mouse, as has been observed in human skin inflammatory disorders. With administration of antibodies 16, 116, 111 and non-significantly 110, skin LC count was diminished in the prevention model (FIG. 6A). Notably, antibody 116 reduced skin LC numbers below those in the wild-type skin showing an improved and surprising level of effect. As the predominant CD1a expressing population, the effect of antibodies on LC CD1a expression was assessed. It was of note that antibodies 110 and 116 had reduced staining, but this was due to interference of the 110/116 antibodies to binding by the HI149 detection antibody (FIG. 6B). This shows sustained binding of the antibodies in vivo which is a surprising effect and is associated with therapeutic advantage. The findings also raise the possibility of using the antibodies for diagnostic or prognostic purposes or monitoring CD1a-expressing cells before and during treatment. This observation was not seen with a non-competing SK9 detection antibody as presented below. The observed LC reduction could be due to antibody-dependent LC death or migration or altered phenotype. As such the cervical lymph nodes were analysed for presence of CD11c+ Langerin+ LCs. It was found that an increased number of LCs in the lymph node of CD1a transgenic mice, compared to WT, however migration to the LN did not appear to explain the reduction in skin LCs for mice treated with antibodies 110 and 116 (FIG. 6C). Notably, antibody 116 brought immunological improvements close to those in the wild-type skin showing an improved and surprising level of effect. Interestingly the level of expression of CD1a on the lymph node-derived LCs followed a similar pattern to that of the skin, in that LC had reduced staining, which was due to interference of the 110/116 antibodies to binding by the HI149 detection antibody (FIG. 6D) as discussed further below. This was not seen with a non-competing SK9 detection antibody. It is of note that the lymph node derived LCs expressed less CD1a per cell than those of the skin, this may be a control mechanism to prevent systemic inflammation. The antibodies therefore maintain effects on LC in vivo in the skin and even after migration to the lymph nodes. This is an important enhancement as the clinical effects will be more long-lasting.

### Example 5—Anti-CD1a Antibody Observed Cytotoxicity Expressed in Effects on CD1a-Expressing Cell Phenotype

Given that enhanced migration did not fully explain skin LC reduction, the potential for antibody induced alterations in phenotype of CD1a+ cells was investigated, despite the murine IgG1 nature.

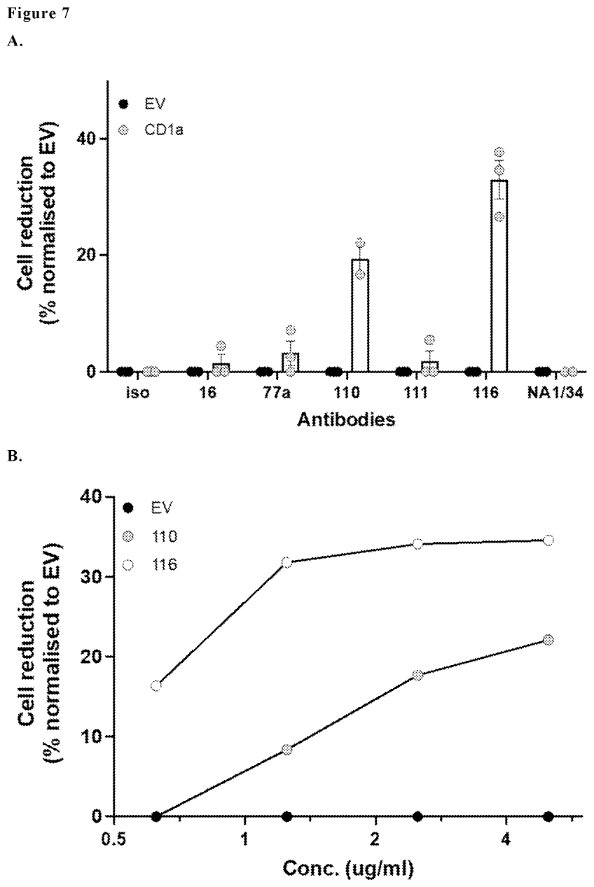

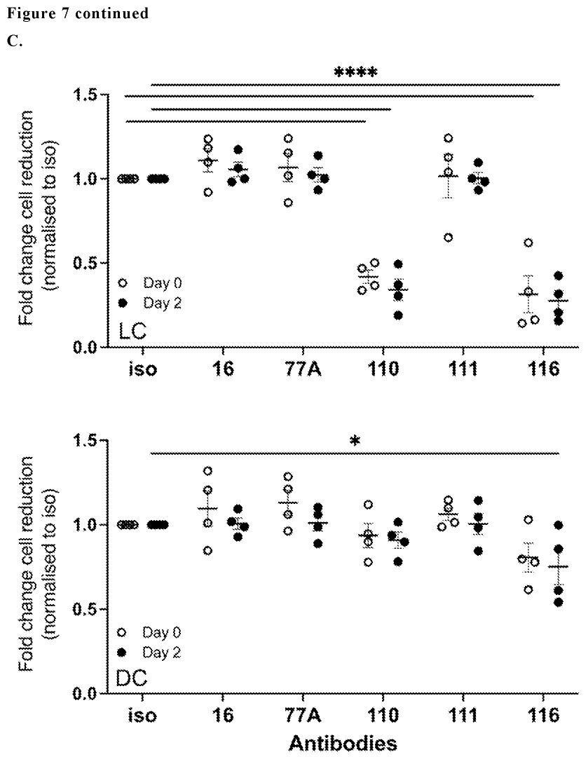

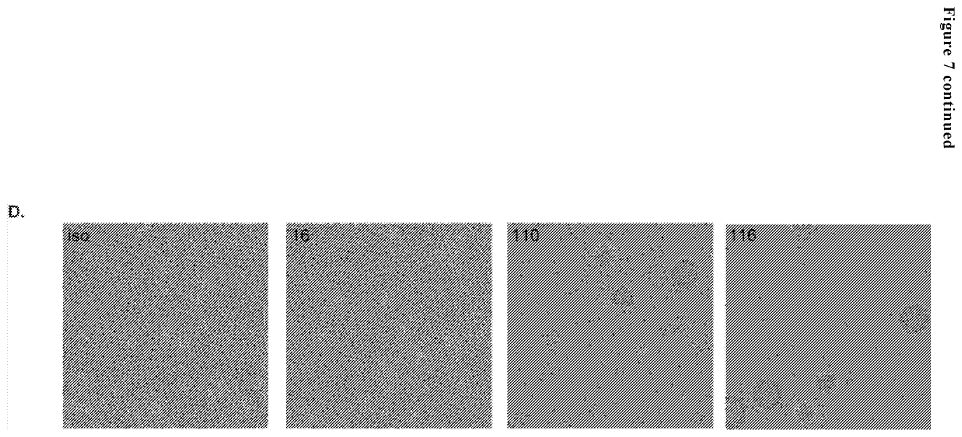

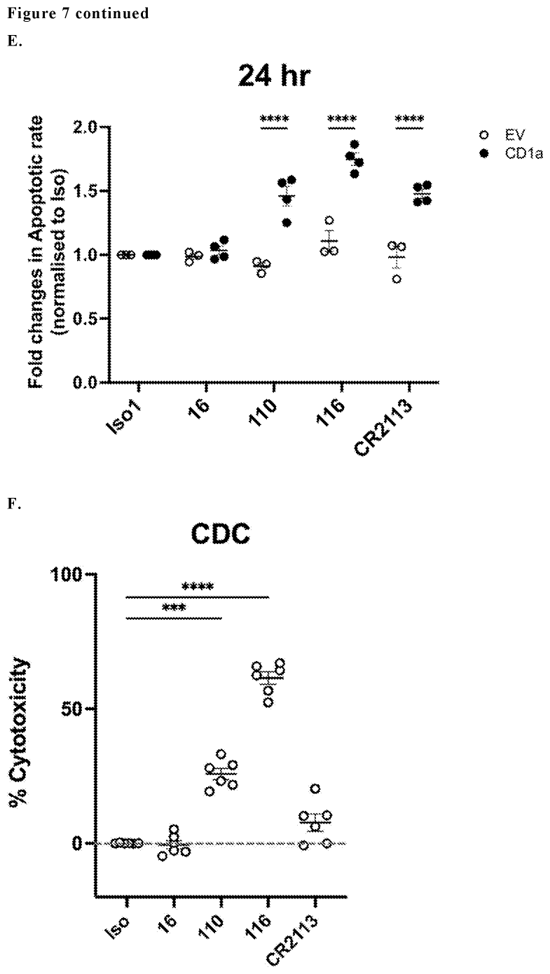

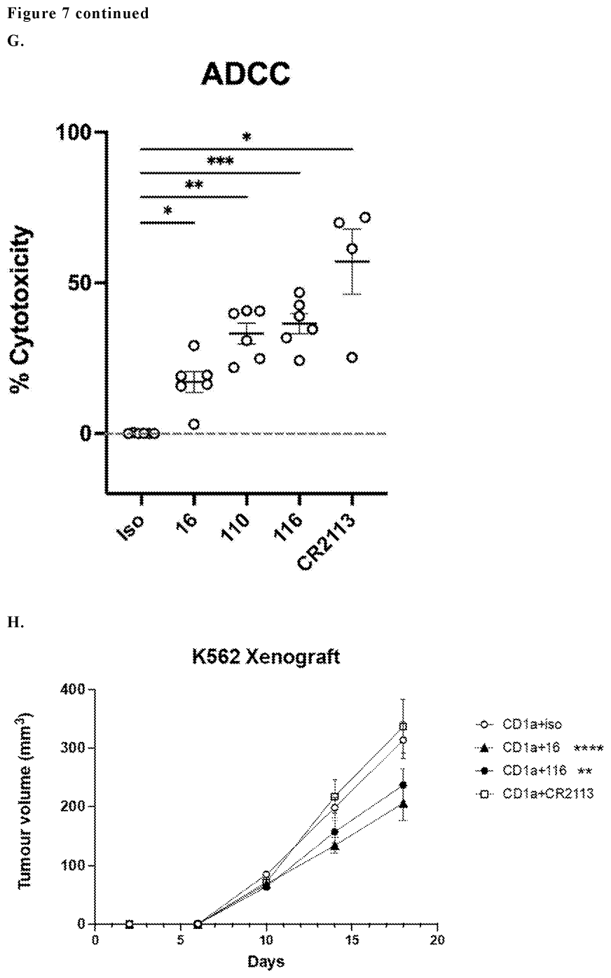

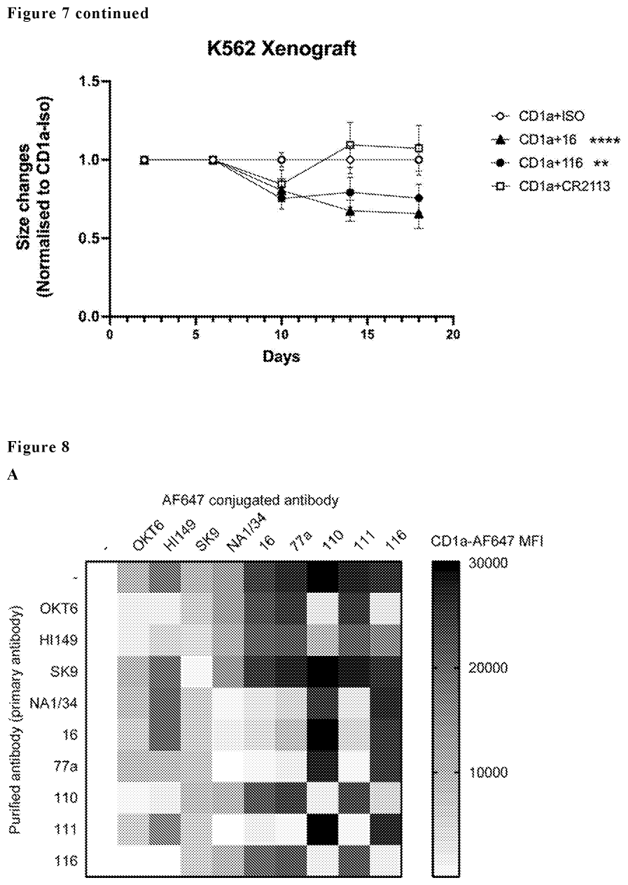

It was demonstrated that all anti-CD1a antibodies, but in particular 110 and 116, were capable of in vitro reduction in number of CD1a+ K562 cells which lack MHC class I and II and so permit comparison of responses (FIG. 7A). Antibodies 110 and 116 were tested in more detail which showed reduction in a dose dependent manner (FIG. 7B) which was an improvement and a surprise given the IgG1 isotype. This was apparently different to the published CR2113 antibody (16, 18) (U.S. Pat. No. 10,844,118B2 and CA 2924882 A1) which is stated to require complement and/or antibody-dependent cellular cytotoxicity. Specifically, it is stated “CR2113 does not directly induce apoptosis” (17) and it was noted that NA1/34 does not induce direct killing. However, as different Fc regions influence effector functions, the comparative effects of CR2113 on a murine IgG1 background are addressed directly below. The inventors went on to assess the capacity of the antibodies to induce direct reduction of primary human CD1a expressing cells. DC- and LC-like cells were generated through 5 day in vitro differentiation of monocytes using cytokines IL-4/GM-CSF, and IL-4/GM-CSF/TGF-B respectively with the addition of anti-CD1a antibodies on day 0 or 2 of culture. It was observed that antibodies 110 and 116 reduced LCs and to a lesser extent DCs in vitro (FIG. 7C upper and lower panel respectively). In exploration of the mechanisms underlying this reduction, the inventors found the reduction to be associated with a striking cell clustering culture phenotype morphology (FIG. 7D). The reduction in number could be partly explained by this clustering, but in addition, it was tested whether the antibodies could induce apoptosis of CD1a-expressing target cells and compared to CR2113 (on murine IgG1 background). FIG. 7E shows that 110 and 116 (but not 16) and CR2113 (on murine IgG1 background) induce annexin V expression by CD1a-expressing K562, even in absence of complement or ADCC. This suggests that 110, 116 and CR2113 antibodies can mediate K562 cell death to some extent. In order to investigate the role of complement-mediated lysis (CDC) and antibody-dependent cytotoxicity (ADCC), K562-CD1a were incubated with complement (FIG. 7F) and/or with human PBMC (FIG. 7G). Despite the murine IgG1 nature of the antibodies, there was evidence of complement-mediated lysis and ADCC. To further investigate mechanisms in vivo, a new model was established using K562-CD1a subcutaneous tumours in an immunodeficient NSG model where there are broadly deficient lymphocyte responses and other effects. The data showed that all three antibodies reduced the size of the lymphoid cell tumours by day 10, with the effects sustained (to at 25% or greater reduction in CD1a-expressing tumour cell volume) for 16 and 116 by days 15-20 but lost for CR2113 (FIG. 7H). The differences in in vitro and in vivo responses may be explained by other cofactors present in vivo such as complement, numerous innate cell subsets bearing FcR with specificity for different Fc, differential target cell density, reduced antibody half-life in vivo, and altered tissue access. Such a direct alteration of phenotype of CD1a-expressing target cells may facilitate a less inflammatory response of CD1a-expressing cells. As such, the reduction of LCs in the skin of CD1a-Tg mice treated with 110 and 116 may be partly explained by direct antibody dependent change in phenotype of CD1a+ LCs and contribute to the clinical effect, for example in 116 reducing inflammation to below that of wild-type. The data also raise the possibility that the antibodies may have utility in treatment of CD1a-expresing malignancies which include Langerhans cell histiocytosis and some forms of T cell lymphoma and some forms of thymoma. However, phenotypic alteration of target cells does not explain the reduction of T cell functional responses shown in FIG. 2, as the CD1a-bead assay (FIG. 2C) would not be affected by any depletion effects.

### Example 6—Epitope Binding Analysis of CD1a Antibodies

The data presented herein demonstrates that the five newly generated anti-CD1a antibodies have a range of functionality and it was sought to determine whether the antibodies have overlapping binding sites, using a flow cytometry cross-blocking assay. Additionally, epitope overlap was assessed with commercially available antibodies OKT6, HI149, SK9 and NA1/34 (binding site known to overlap with CR2113, as above).

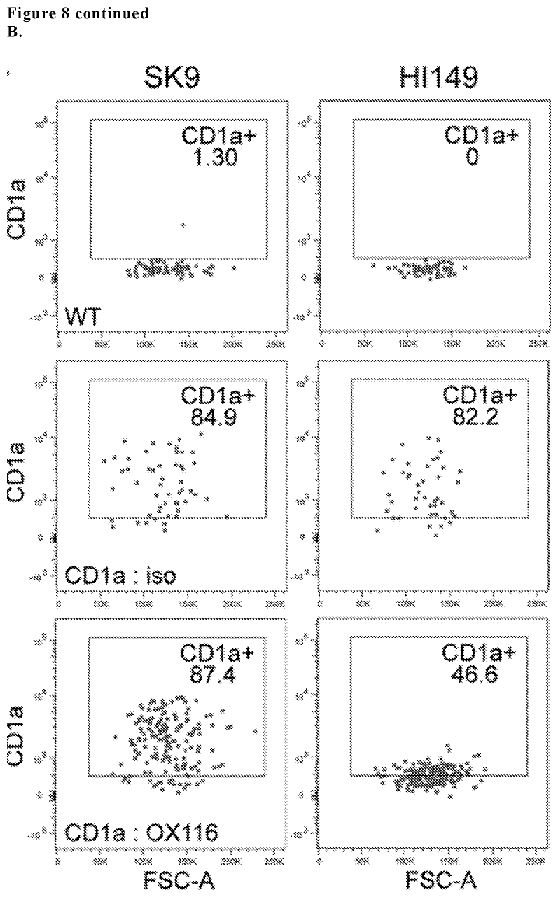

CD1a-K562 cells were incubated with purified primary anti-CD1a antibodies (Y axis FIG. 8A, 25 μg/ml), the unbound antibody was then washed away and Alexa-Fluor-647 conjugated forms of the different antibodies were then incubated with the cells in the matrix arrangement of FIG. 8A (X axis, 10 μg/ml). Mean fluorescent intensity (MFI) was used to assess the degree of binding of the fluorophore conjugated antibody and so any steric interference caused by binding of the primary purified antibody would be represented by a decrease in MFI. The results indicated that antibodies HI149, OKT6, 110 and 116 may have overlapping or closely associated epitopes and a second group containing antibodies NA1/34, 77a, 111 and 16 may have closely related binding sites. This suggests the reduction in CD1a expression observed in vivo (FIGS. 6B and D) was due to interference of the 110/116 antibodies to binding by the HI/149 detection antibody. Indeed, this effect was not seen with a non-competing SK9 detection antibody (FIG. 8B). Importantly and unexpectedly, the antibodies therefore maintain presence on LC in vivo in the skin and even after migration to the lymph nodes and following skin tissue enzymatic digestion. This will likely associate with a more prolonged and substantial clinical benefit. As the antibodies fall into two main groups which do not compete, FIG. 8 (A and B) shows that combinations of antibody members selected from each group can be used together, for example as therapeutic/monitoring or combined therapeutics. One such combination would be 116 and 16.

### Example 7—Demonstration of Effectiveness of Antibodies of the Invention on Treatment of Imiquimod-Induced Inflammation and Also Systemic Associated Inflammation

Given the skin-dominant expression of CD1a, most studies have focused on skin-specific functional effects, although the presence of circulating CD1a-reactive T cells has been demonstrated (11). A role for CD1a in inflammation of tissues beyond the skin has not been extensively studied. Furthermore, CD1a is known to amplify the imiquimod skin response (16), but there have been no studies on associated systemic sequelae. The inventors generate a novel CD1a transgenic mouse and CD1a-reactive T cells, and characterize anti-CD1a antibodies for functionality in vitro and in vivo using human and mouse assays respectively. The findings confirm CD1a-dependent effects extend to systemic effects, with implications for treatment of systemic associations of skin disease including adverse inflammatory drug reactivity.

### Therapeutic Potential of Anti-CD1a Antibodies

To further evaluate the therapeutic potential of the newly generated anti-CD1a antibodies, the inventors tested the three most clinically effective antibodies 16, 110 and 116 in an imiquimod treatment model, where the anti-CD1a antibodies were introduced after the establishment of imiquimod-induced inflammation (FIG. 9A). All three antibodies improved clinical responses rapidly after initiation despite ongoing imiquimod application (FIG. 9B-C). The responses were most marked for 116, which reduced ear thickness (FIG. 9B). Whole skin (upper panel) and epidermal (lower panel) thickening was visualised by confocal microscopy (FIG. 9D), which confirmed the micrometer assessment (FIG. 9B). CD1a protein expression was assessed (anti-CD1a OKT6 AF-594, red) in the CD1a transgenic epidermis and noted to be reduced, through cell death and epitope competition, in 110 and 116 treated skin (FIG. 8A and FIG. 9D). Upon analysis of the cutaneous cellular immune response following the imiquimod treatment model we observed reduced skin T cell count and activation, reduced skin LCs, and reduced skin neutrophils after introduction of the antibodies (FIG. 9E-G).

### CD1a is Involved in the Systemic Immune Reaction to Imiquimod

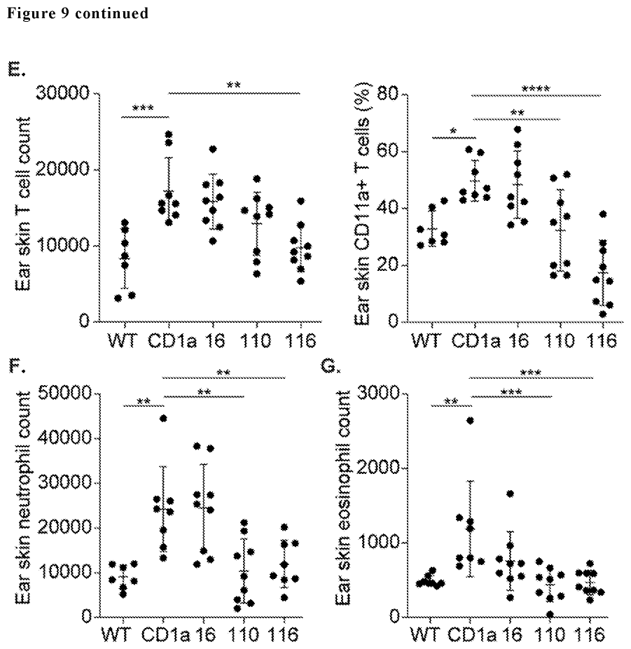

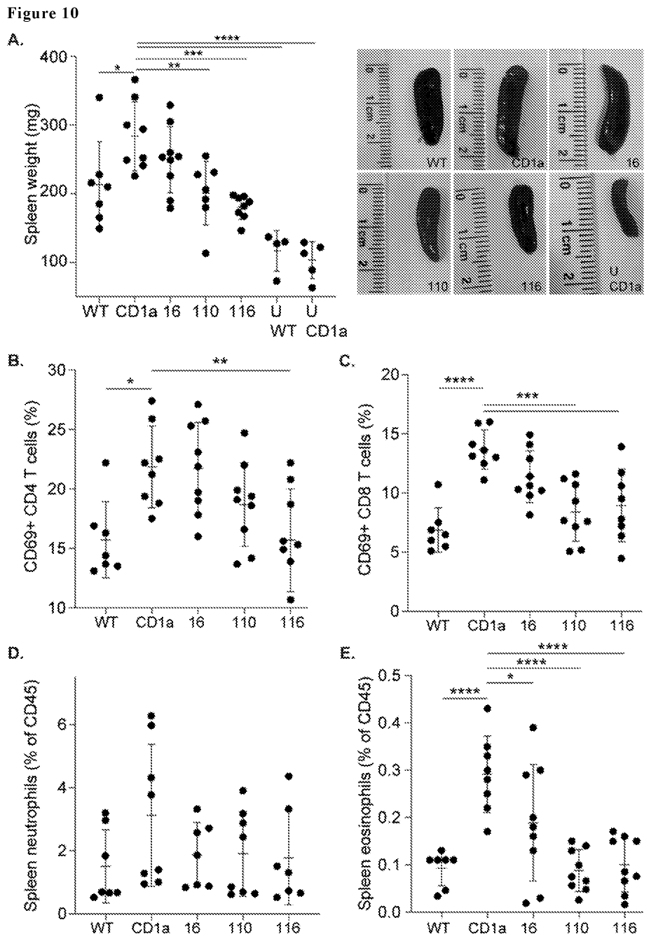

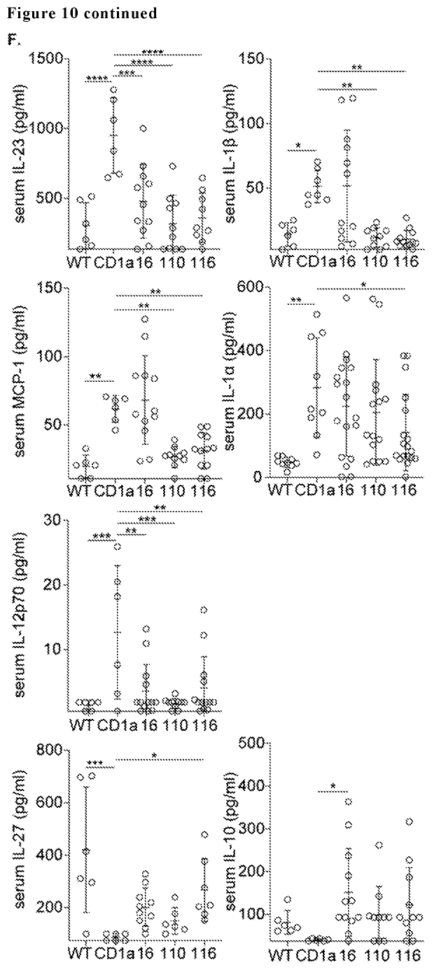

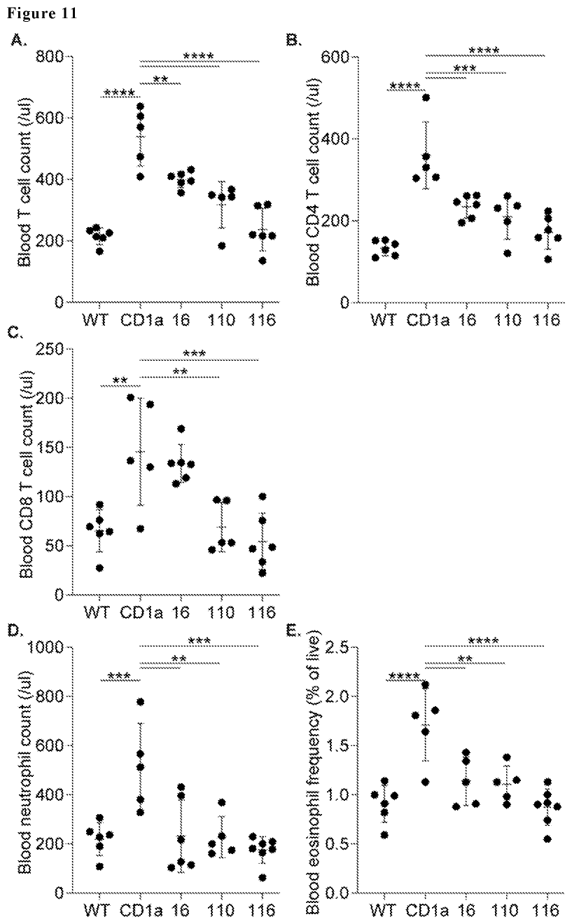

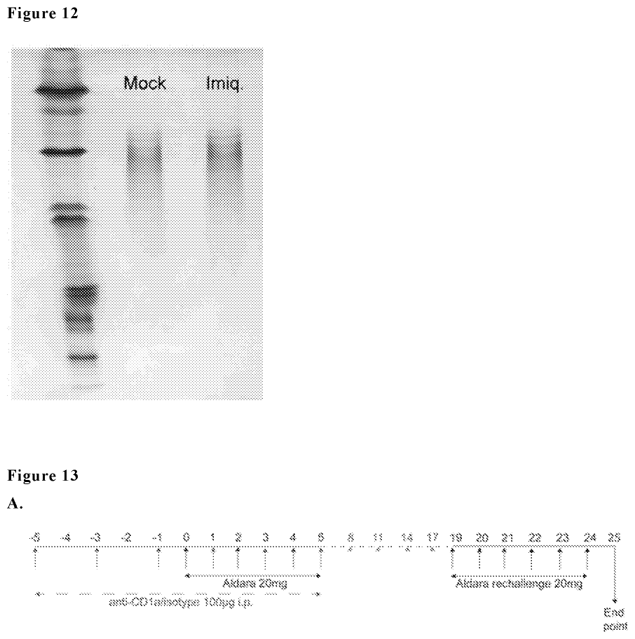

The human effects of imiquimod treatment can extend beyond the skin, and in the murine model have been shown to induce splenomegaly. The contribution of CD1a to this pathway was evaluated. Strikingly, spleen weight was increased in the imiquimod treated CD1a Tg mouse compared to wild-type and the antibodies reduced spleen size and weight, consistent with systemic effects beyond the skin (FIG. 10A). Furthermore, the antibodies reduced CD4 and CD8 T cells activation as determined by CD69expression (116 and 110, FIG. 10B-C), splenic neutrophil (non-significant trend) and eosinophil frequencies (16, 110, 116) (FIGS. 10D and 10E respectively). Plasma cytokine levels were assessed at day 8. Significant increases in IL-23, IL-12p70, IL-1β, IL-1α and MCP-1 were observed in the imiquimod treated CD1a transgenic mice, and were reduced in some or all of the 16, 110 and 116 treated groups (FIG. 10F). Plasma immunoregulatory cytokines IL-10 and IL-27 were increased in the presence of the antibodies 16 and 116 respectively (and trend with the others). The impact on circulating immune cells was then ascertained. Similar to the spleen, blood CD4 and CD8 T cell counts, neutrophilia and eosinophilia were increased in the imiquimod-treated CD1a transgenic group. This increase was significantly blocked following treatment with 16,110 or 116 (FIG. 11A-E). Lastly, the inventors investigated whether imiquimod itself might be a CD1a ligand and showed that this is not the case, implicating wider autoimmune and autoinflammatory effects of the CD1a pathway (FIG. 12). Therefore, it can be suggested that broad systemic inflammatory immune responses are primed or influenced by CD1a in the skin.

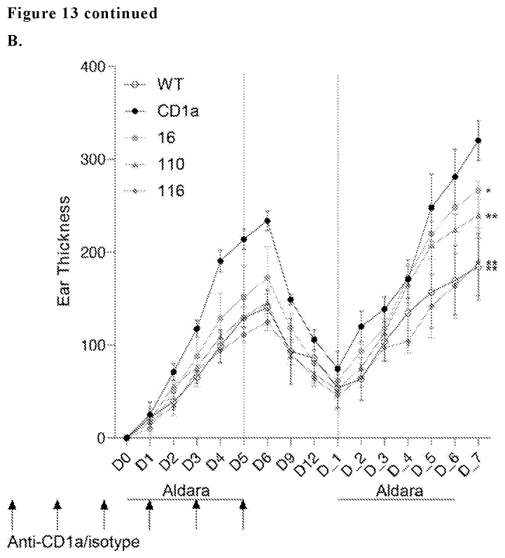

In order to investigate whether the anti-CD1a antibodies could produce a sustained reset of skin inflammation following imiquimod application, the model depicted in schematic FIG. 13A was undertaken where imiquimod re-challenge was used in the absence of re-administration of the anti-CD1a antibodies (FIG. 13B). Surprisingly, 16, 110 and 116 all produced sustained improvement in ear thickness in the absence of repeat antibody administration, consistent with a sustained immunological effect. The immunological response was also sustained with significant reductions in the frequency of skin T cells (110, 116), skin T cell activation (16, 110, 116), skin eosinophils (116) and skin neutrophils (16, 110, 116), lymph node T cell frequency (110, 116), lymph node T cell activation (16, 116), lymph node Langerhans cells (116), lymph node eosinophils (116) and lymph node neutrophils (116), blood T cell frequency (110, 116), blood T cell activation (116), blood eosinophils (110, 116), plasma IL-1α (116), IFNγ (16, 110, 116), IL-1B (16, 110, 116), IL-6 (16, 116), IL-17A (16, 110, 116).

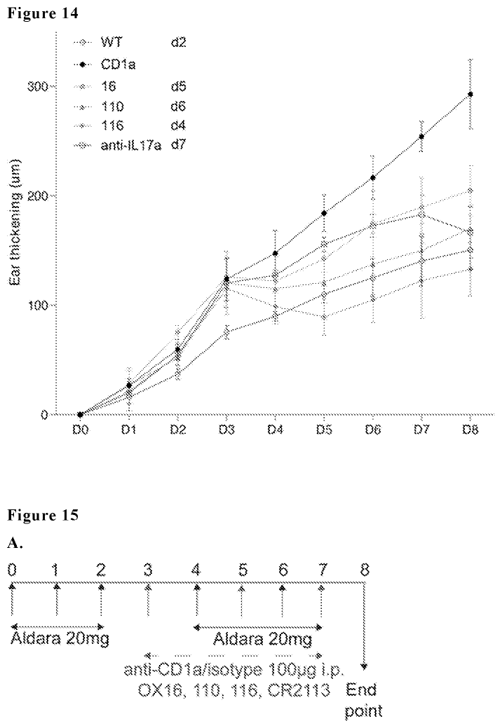

In order to compare performance of the antibodies with a current standard of care in the management of moderate-severe psoriasis, the imiquimod treatment model (FIG. 9A) was repeated alongside anti-IL-17A (IgG1 isotype) administered at the same time and dose (100 μg) as the anti-CD1a antibodies (FIG. 14). All anti-CD1a antibodies again showed significant improvement in ear thickness outcomes, with all producing significant improvements earlier than anti-IL-17A. It was noted that in contrast to the different anti-CD1a antibodies, the anti-IL-17A did not significantly reduce frequency of skin T cells, skin Langerhans cells, skin eosinophils, lymph node T cells, lymph node neutrophils, lymph node eosinophils, plasma IL-23, MCP-1, IL-6.

In order to directly compare skin and systemic inflammatory outcomes between the antibodies described herein and CR2113, the imiquimod skin treatment model was undertaken (FIG. 15A). All anti-CD1a antibodies had a beneficial effect on ear thickness, but antibody 116 was significantly improved over CR2113 (FIG. 15B-C). To extend the investigation of the improvement of the anti-CD1a antibodies 16,110 and 116 over CR2113, a comparison was made for an additional model of skin inflammation, namely MC903-induced inflammation (FIG. 15D) and a significant benefit was observed for antibodies 16, 110 and 116, but not CR2113, thus showing an improvement (FIG. 15E). It was noted that 16 and 116 showed a significant reduction in skin T cell percentage and skin eosinophil count, whereas CR2113 did not show significant reduction (FIG. 15F). Skin extract cytokines were significantly reduced where CR2113 did not show significant reduction for IL-5 (16, 110, 116), IL-6 (16, 110, 116), IL-9 (16), IL-23 (116), IL-17F (16, 110, 116).

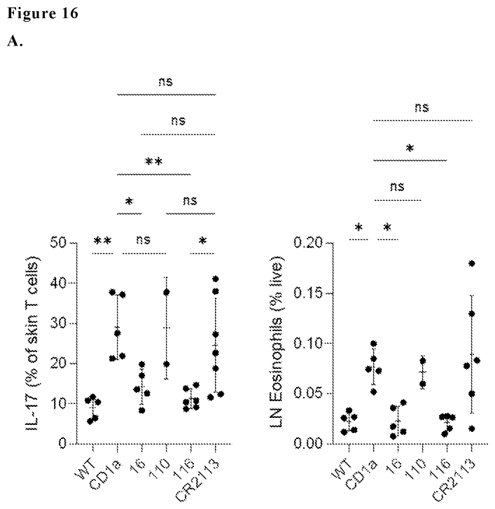

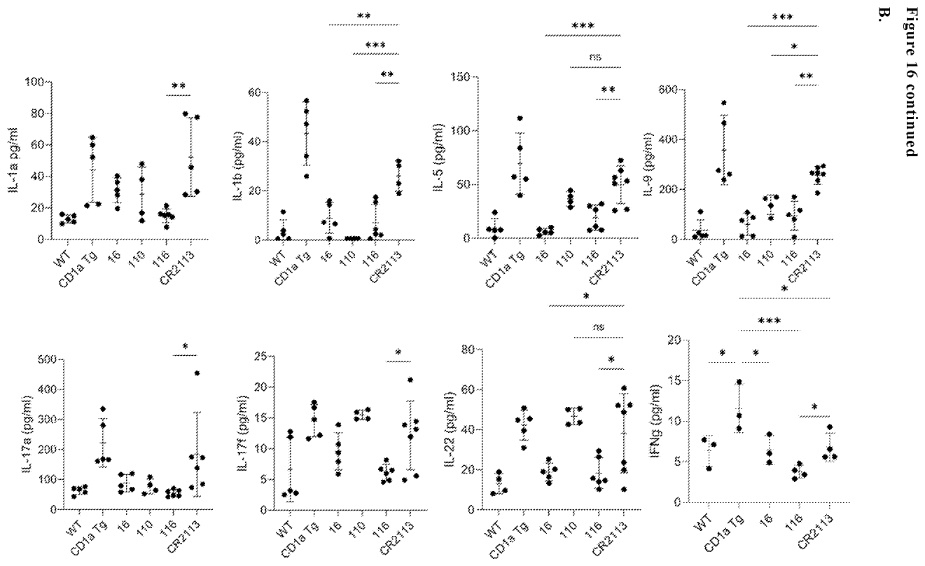

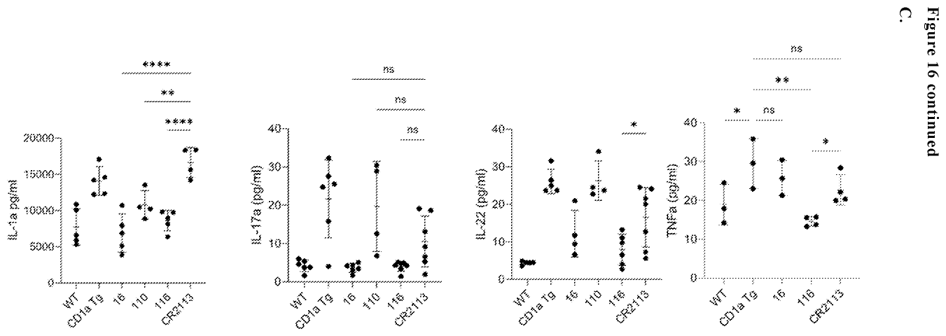

It was further observed that 116 showed consistent improvement over CR2113 in reducing skin, lymph node and plasma inflammatory responses to imiquimod (FIG. 16). For some outcomes, 16 was also significantly improved over CR2113 (FIG. 16). Specifically, antibody 116 was improved over CR2113 in reducing IL-17A expression by skin T cells, and in the frequency of draining lymph node eosinophils. 116 was also improved over CR2113 in reducing plasma IFNγ, IL-1α, IL-1B, IL-5, IL-9, IL-17A, IL-17F, IL-22 and skin digest IL-1α, IL-22 and TNFα. 16 was improved over CR2113 in reducing lymph node eosinophils, plasma IL-1β, IL-22, IL-9 and IL-5; and skin digest IL-1α and strong trends in IL-17A. Overall, the data confirm that the antibodies described herein are able to inhibit skin and systemic inflammatory responses to imiquimod and MC903.

### Discussion

Skin inflammation such as dermatitis, psoriasis and lupus are common disorders with significant associated physical and psychological morbidity. Cutaneous adverse reactions to drugs are also common, ranging at 1.8-7 per 1000 hospitalised patients. Severe cutaneous adverse reactions, with widespread and systemic effects such as SJS, TEN, AGEP and DRESS are less common; for example, SJS/TEN has an incidence of approximately 1-6 cases per million individuals per year (M. Mockenhaupt, Allergol Select 1, 96-108 (2017)). Gell and Coombs defined a classification of hypersensitivities in the 1960s in which delayed type IV hypersensitivity required a role for effector T cells (R. R. A. Coombs, Gell, P.G.H., Classification of allergic reactions responsible for drug hypersensitivity reactions. In Clinical Aspects of Immunology. (Davis, Philadelphia, ed. second, 1968)). Although there is increasing recognition that the classification cannot account for all aspects of drug hypersensitivity, there has still largely been a focus on altered recognition of covalent haptens or non-covalently modified peptide/MHC molecules. However, the current models do not explain the dominance of skin and mucosal involvement of drug hypersensitivity (M. Mockenhaupt, Allergol Select 1, 96-108 (2017).

Through generation of a CD1a transgenic mouse and autoreactive human CD1a restricted enriched T cell lines, and characterisation of functional anti-CD1a antibodies, the data presented here show induction of CD1a presentation of endogenous lipid ligands. This leads to an autoreactive T cell-mediated cutaneous and systemic inflammation. The anti-CD1a antibodies had clinical and immunological effects, whether they were blocking or blocking/modulating, suggesting that CD1a lipid presentation to T cells is of importance. TLR7 can recognize single stranded RNA, and so it is of interest that reactivity to viral infections can mimic the clinical phenotype of different severe forms of cutaneous inflammation including psoriasis, dermatitis, lupus and adverse inflammatory reactions to drugs, including SJS and TEN. Such shared final common clinical manifestations might indicate that a number of precipitants can promote CD1a-autoreactivity and auto-inflammation. The model might also help explain the increased risk of autoimmunity associated with certain drug reactions, including lupus erythematosus and DRESS syndrome. Furthermore, the findings would implicate CD1a-autoreactivity in the breaking of wider T cell tolerance.

In addition to effects on the T cell response to the imiquimod-containing drug Aldara, increased neutrophil and eosinophil responses in the skin, draining lymph node and spleen were observed in the CD1a transgenic mouse. These effects were inhibited by the administration of antibodies of the invention, in particular 16, 110 and 116. This implicates a CD1a-dependent immune cascade that is wider reaching that initially anticipated. Neutrophil depletion has been shown to ameliorate the severity of imiquimod-induced inflammation (H. Sumida et al., Interplay between CXCR2 and BLT1 facilitates neutrophil infiltration and resultant keratinocyte activation in a murine model of imiquimod-induced psoriasis. J Immunol 192, 4361-4369 (2014).

Aldara/imiquimod application recapitulates key aspects of different forms of skin inflammation and associated systemic diseases and disorders, including psoriasis, dermatitis, lupus and severe cutaneous hypersensitivity reactions including T cell and neutrophil infiltration as discussed above. The data demonstrated herein shows that imiquimod-dependent eosinophil infiltration of the skin, lymph nodes and spleen was enhanced in the CD1a-transgenic mouse and reduced by administration of antibodies of the invention, in particular 16, 110 and 116.

Furthermore it has been reported that LC numbers are increased in lesional skin compared to non-lesional skin of patients with different forms of inflammatory skin diseases or disorders including psoriasis, dermatitis, lupus; and a maculopapular drug eruption, and were decreased to non-lesional levels as the eruption resolved (D. I. Dascalu, Y. Kletter, M. Baratz, S. Brenner, Acta Derm Venereol 72, 175-177 (1992)). Interestingly, psoriasis is associated with altered LC migration, suggesting that although imiquimod application is a well-studied and effective murine model of psoriasis and lupus and dermatitis, it also has applicability to include adverse drug inflammatory drug reactions. Here, the inventors show that CD1a-antibody dependent modulation of LCs was associated with reduced skin inflammation upon administration of antibodies of the invention, in particular 110 and 116, which may be of therapeutic importance to the treatment of psoriasis, dermatitis, lupus, inflammatory drug reactions and other conditions. The epitope analysis highlights the potential therapeutic importance of epitope binding site; the anti-CD1a antibodies fell into two groups based on binding site and resultant effector function. The epitope site may facilitate the clustering and change in phenotype effect seen with antibodies 110 and 116, but not 77a, 111 and 16, which were primarily blocking antibodies. The clustering may indeed lead to cross-linking/agglutination-like cell morphology, which may also explain the reduction of CD1a-transfected K562 and monocyte derived LCs as both cell types express high levels of CD1a, higher than monocyte derived DCs. The different antibody binding sites of the two groups do not compete and so there is utility for combinations selected from each of the two groups, for example in therapeutics/monitoring or in combination therapies.

The role of CD1a in the pathogenesis of skin inflammation and associated systemic disease implicates its role in many diseases, including psoriasis, dermatitis and lupus erythematosus and drug hypersensitivity. Furthermore, characterization of CD1a blocking and modulating antibodies offers a new potential route to preventative and therapeutic development for skin inflammation and CD1a-expressing malignancies.

### Summary

In summary, the inventors have generated a refined panel of anti-CD1a antibodies with therapeutic potential in the prevention and/or treatment of inflammatory skin and mucosal disorders. The five antibodies 16, 77a, 110, 111 and 116 were shown to be potent inhibitors of in vitro human CD1a antigen presentation and showed efficacy in exemplar inflammatory skin disease prevention and treatment models which have features of psoriasis, dermatitis, lupus erythematosus and drug reactions which manifest as an inflammatory skin or mucosal disease or disorder, as well as those which are systemic (non-cutaneous), and in a xenograft tumour model. The success of the antibody discovery process in identifying improved antibodies may be attributed to combining: a) the screening of large numbers of hits (3500) with; b) the use of the novel chimeric immunogen, whereby the human CD1a lipid binding domain was fused to the host organism CD1d Ig domain, thus targeting antibody generation to the lipid binding domain where functional inhibition potential may lie with; c) a variety of polyclonal and enriched T cell analyses examining different functional outcomes.

In vitro human functional assays showed the antibodies to be more potent than commercially available antibodies, measured by IC50 assessment of inhibition of a primary polyclonal T cell response. Furthermore, using highly sensitive human CD1a-restricted T cell clonal assays, it was determined that anti-CD1a antibodies 16 and 116were capable of blocking IL-22 production, which is a key regulator of inflammatory skin and mucosal disease. Such an activity was an improvement and surprise as this was not shown in existing publications or patents of anti-CD1a CR2113 ((16, 17), U.S. Pat. No. 10,844,118B2 and CA 2924882 A1), where IL-17 or IFNγ production was induced and inhibited in the murine system. IL-22 inhibition is an important advantage of the antibodies as IL-22 is a key regulator of skin and mucosal disease.

The parallel analyses of human and in vivo murine models provide a powerful means to assess the therapeutic benefit of the newly generated antibodies. In vivo, imiquimod was utilised to induce a psoriasis-like, dermatitis-like, lupus-like, drug-reaction-like phenotype and provide a model skin inflammation system, and may be more widely applicable to a number of inflammatory diseases and disorders as well as for associated systemic diseases or disorders and inflammatory drug reactions which manifest systemically. Here it was shown that antibodies 110, 116 and 16 significantly reduce the CD1a-dependent inflammation induced by imiquimod, with improvements over standard of care (anti-IL-17A) and a comparator anti-CD1a antibody on the same murine IgG1 background (CR2113). Importantly, and unexpectedly, antibody 116 reduced the skin inflammation below that of the WT imiquimod-treated mice, and normalised many of the skin and systemic immunological markers to that of WT, suggestive of a mechanism by which anti-CD1a 116 has effects beyond the inhibition of CD1a-TCR signalling. The skin was immunophenotyped and reduction in T cell numbers and activation was observed, as was neutrophil infiltration to the WT level with administration of antibodies 110, 116 and 16. Observation of reduced neutrophilia to the WT level is an unexpected improvement upon published anti-CD1a CR2113,highlighting the potential of antibodies 110, 116 and 16.

Importantly when the LC population within the skin was analysed, significant reduction in the CD11c+Langerin+ LCs was observed following administration of the antibodies 110 and 116. This reduction was not explained by enhanced migration to the draining lymph node. It is however possible that the antibodies 110 and to a greater extent 116 are capable of directly reducing CD1a+ cells in vivo, explaining the reduction in skin LCs in vivo and evidenced by the striking reduction of human CD1a+ cells in vitro. This is a surprising result given the mouse IgG1 isotype of the antibodies-where a murine IgG2a isotype is more likely to lead to cytotoxicity via complement-mediated lysis or antibody-dependent cellular cytotoxicity, and further patented and published anti-CD1a CR2113 has been reported not capable of direct depletion (17), although here it was shown that apoptosis of CD1a-expressing cells could also be induced by CR2113 on a murine IgG1 background. The modulation ability of these antibodies could help explain the reduction of imiquimod induced inflammation below that of WT isotype treated mice. Antibody 116 not only blocks the interaction of CD1a with the TCR but also modifies LCs reducing/resetting the inflammatory potential of the skin and normalised many of the skin and systemic immunological markers to that of WT. This may explain the ameliorating effect over and above the CD1a-dependent response to improvement beyond wild-type, which anti-CD1a CR2113 does not.

Furthermore, the data suggest that the 16, 110 and/or 116 antibodies presented here have utility in the treatment of CD1a-expressing malignancies such as Langerhans cell histiocytosis or some forms of T cell lymphoma and thymomas. This may be by direct effects or wherein an anti-CD1a antibody is coupled or associated with one or more other therapeutic agent is selected from the group comprising cytotoxic agents, anti-inflammatory agents such as steroids, and CAR-T cells such as regulatory or cytolytic CAR-T cells, or other cells expressing or presenting the antibody or antigen binding fragment.

This investigation demonstrates antibody 16 as a highly effective blocking antibody ablating CD1a dependent inflammation in vivo without inducing direct apoptosis, 110 modifies LC phenotype and function, significantly reducing CD1a dependent inflammation in vivo, and 116 is a highly effective blocking and modifying antibody which reduces inflammation below the WT level and normalised many of the skin and systemic immunological markers to that of WT. This grouping of antibodies is consistent with the basic epitope analysis where directly modifying antibodies 110 and 116 cluster and blocking antibodies 77a, 111 and 16 cluster. The epitope analysis also revealed group 77a, 111 and 16 overlapped with the epitope recognised by non-depleting NA 1/34; this is important to note as NA1/34 has been shown to cross-block binding of anti-CD1a CR2113. Antibodies 110 and 116 did not cross-block NA1/34 and therefore likely represents a different epitope region. The antibodies maintain presence on LC in vivo in the skin and even after migration to the lymph nodes. This is an important enhancement as the clinical effects will be more long-lasting.

With these data the inventors demonstrate the potential of this refined panel of improved anti-CD1a antibodies in the prevention and treatment of inflammatory skin and mucosal conditions including, but not limited to, psoriasis, dermatitis, lupus as well as for use in treating and/or preventing one or more associated systemic diseases or disorders, or one or more inflammatory drug reactions which manifest systemically. The effects on a wide cascade of inflammation including LC, T cells and neutrophils, particularly of antibodies 110, 116 and 16, would have wide reaching effects in inflammatory skin and mucosal disorders including psoriasis, dermatitis, lupus and drug reactions which manifest as an inflammatory skin or mucosal disease or disorder, or CD1a-expressing malignancies.

In conclusion the inventors demonstrate improved anti-CD1a antibodies 16, 77a, 110, 111 and 116 as a method for preventing and treating inflammatory skin and mucosal diseases or disorders, or as associated systemic diseases or disorders, or inflammatory drug reactions which manifest systemically, or CD1a-expressing malignancies through blocking of CD1a and/or modifying the phenotype/function of CD1a+ cells.

### REFERENCES

1. G. F. Murphy, A. K. Bhan, S. Sato, M. C. Mihm, Jr., T. J. Harrist, A new immunologic marker for human Langerhans cells. N Engl J Med 304, 791-792 (1981).
2. Y. L. Chen et al., Re-evaluation of human BDCA-2+ DC during acute sterile skin inflammation. J Exp Med 217, (2020).
3. S. G. Turville et al., Diversity of receptors binding HIV on dendritic cell subsets. Nat Immunol 3, 975-983 (2002).
4. M. Alcantara-Hernandez et al., High-Dimensional Phenotypic Mapping of Human Dendritic Cells Reveals Interindividual Variation and Tissue Specialization. Immunity 47, 1037-1050 e1036 (2017).
5. C. S. Hardman et al., CD1a presentation of endogenous antigens by group 2 innate lymphoid cells. Sci Immunol 2, (2017).
6. E. L. Reinherz, P. C. Kung, G. Goldstein, R. H. Levey, S. F. Schlossman, Discrete stages of human intrathymic differentiation: analysis of normal thymocytes and leukemic lymphoblasts of T-cell lineage. Proc Natl Acad Sci U S A 77, 1588-1592 (1980).
7. D. B. Moody et al., Structural requirements for glycolipid antigen recognition by CD1b-restricted T cells. Science 278, 283-286 (1997).
8. L. Gapin, D. I. Godfrey, J. Rossjohn, Natural Killer T cell obsession with self-antigens. Curr Opin Immunol 25, 168-173 (2013).
9 T. Mallevaey et al., A molecular basis for NKT cell recognition of CD1d-self-antigen. Immunity 34, 315-326 (2011).
10. K. S. Wun et al., T cell autoreactivity directed toward CD1c itself rather than toward carried self lipids. Nat Immunol 19, 397-406 (2018).
11. A. de Jong et al., CD1a-autoreactive T cells recognize natural skin oils that function as headless antigens. Nat Immunol 15, 177-185 (2014).
12. E. Layre, A. de Jong, D. B. Moody, Human T cells use CD1 and MR1 to recognize lipids and small molecules. Curr Opin Chem Biol 23, 31-38 (2014).
13. H. He et al., Tape strips detect distinct immune and barrier profiles in atopic dermatitis and psoriasis. J Allergy Clin Immunol 147, 199-212 (2021).
14. E. G. Langeveld-Wildschut et al., Clinical and immunologic variables in skin of patients with atopic eczema and either positive or negative atopy patch test reactions. J Allergy Clin Immunol 105, 1008-1016 (2000).
15. A. Wollenberg, S. Kraft, D. Hanau, T. Bieber, Immunomorphological and ultrastructural characterization of Langerhans cells and a novel, inflammatory dendritic epidermal cell (IDEC) population in lesional skin of atopic eczema. J Invest Dermatol 106, 446-453 (1996).
16. J. H. Kim et al., CD1a on Langerhans cells controls inflammatory skin disease. Nat Immunol 17, 1159-1166 (2016).
17. G. I. Bechan et al., Phage display generation of a novel human anti-CD1A monoclonal antibody with potent cytolytic activity. Br J Haematol 159, 299-310 (2012).
18. Oteo M, Parra JF, Mirones I, Giménez LI, Setién F, Martinez-Naves E. Single strand conformational polymorphism analysis of human CD1 genes in different ethnic groups. Tissue Antigens 53, 545-50. (1999).
19. Fanti PA, Dika E, Vaccari S, Miscial C, Varotti C. Generalized psoriasis induced by topical treatment of actinic keratosis with imiquimod. Int J Dermatol 45, 1464-5 (2006).
20. Rajan N, Langtry JA. Generalized exacerbation of psoriasis associated with imiquimod cream treatment of superficial basal cell carcinomas. Clin Exp Dermatol 31,140-1 (2006).
21. Patel U, Mark NM, Machler BC, Levine VJ. Imiquimod 5% cream induced psoriasis: a case report, summary of the literature and mechanism. Br J Dermatol. 164, 670-2 (2011).
22. Tedman A, Malla U, Vasanthakumar L, Buzacott K, Banney L. Stevens-Johnson syndrome due to topical imiquimod 5%. Aust J Gen Pract. 49, 662-664. (2020).
23. Yanes DA, Kaffenberger JA, Carr DR. Erythema multiforme as a reaction to imiquimod 5% cream. Dermatol Online J. 23, 13030 (2017).
24. Tandon Y, Brodell RT. Local reactions to imiquimod in the treatment of basal cell carcinoma. Dermatol Online J. 18, 1 (2012).
25. Giraud S, Leducq S, Kervarrec T, Barbarot S, Laghmari O, Samimi M. Spectrum of imiquimod-induced lupus-like reactions: Report of two cases. Dermatol Ther. 33, e13148 (2020).
26. Maxfield L, Gaston D, Peck A, Hansen K. Topical Imiquimod and Subsequent Erythema Multiforme. J Am Osteopath Assoc. doi: 10.7556/jaoa.2020.010. (2019)
27. Hammerl V, Parlar B, Navarini A, Gantenbein L, Väth H, Mueller SM. Mucosal side effects in patients treated with topical imiquimod-A scoping review of the literature. Dermatol Ther. 34, e14355 (2021).
28. Furuoka K, Fukumoto T, Nagai H, Nishigori C. Topical imiquimod-induced lichenoid drug reaction successfully treated with tacrolimus ointment. Dermatol Ther. 33, e14480 (2020).
29. Maguiness SM, Farsani TT, Zedek DC, Berger TG. Imiquimod-induced subacute cutaneous lupus erythematosus-like changes. Cutis. 95, 349-51 (2015).
30. Yokogawa M, Takaishi M, Nakajima K, Kamijima R, Fujimoto C, Kataoka S, Terada Y, Sano S. Epicutaneous application of toll-like receptor 7 agonists leads to systemic autoimmunity in wild-type mice: a new model of systemic Lupus erythematosus. Arthritis Rheumatol. 66, 694-706 (2014).
31. Stockenhuber K, Hegazy AN, West NR, Ilott NE, Stockenhuber A, Bullers SJ, Thornton EE, Arnold IC, Tucci A, Waldmann H, Ogg GS, Powrie F. Foxp3+ T reg cells control psoriasiform inflammation by restraining an IFN-I-driven CD8+ T cell response. J Exp Med. 215, 1987-1998 (2018).

All references cited herein, including patents, patent applications, papers, textbooks and the like, and the references cited therein, to the extent that they are not already, are hereby incorporated herein by reference in their entirety.

TABLE 11
Sequence IDs

| SEQ | | |
|---|---|---|
| ID NO | Feature | Sequence |
| 1 | 16 CDR1 H | GFTFSNYAMS |
| 2 | 16 CDR2 H | AINSNGGSAY YPDTVKD |
| 3 | 16 CDR3 H | RFYYDYGWFA Y |
| 4 | 16 CDR1 L | RASENIDSYL A |
| 5 | 16 CDR2 L | AATLLAD |
| 6 | 16 CDR3 L | QHYYSSPWT |
| 7 | 16-Hv full | EVQLVESGGG LVKPGGSLKL SCAASGFTFS NYAMSWVRQ |
| | | T PEKRLEWVAA INSNGGSAYY PDTVKDRFTI SRDNAKN |
| | | TLY LQMSSLRSED TALYYCARRE YYDYGWFAYW |
| | | GQGTLVTVSS |
| 8 | 16-Lv full | DIVLTQSPAS LSASVGETVT ITCRASENID |
| | | SYLAWYQQKQ GKSPQLLVYA ATLLADGVPS |
| | | RFSGSGSGTQ YSLKINSLQS EDVARYYCQH |
| | | YYSSPWTFGG GTKLEIK |
| 9 | 77a CDR1 H | GFSLSSYAMS |
| 10 | 77a CDR2 H | IISSSGTTYY ASWAKG |
| 11 | 77a CDR3 H | VDYYSSGWGG L |
| 12 | 77a CDR1 L | QASEDIYSNL A |
| 13 | 77a CDR2 L | GASTLAS |
| 14 | 77a CDR3 L | QCTYDTSSYG NT |
| 15 | 77a-Hv full | QSVEESGGRL VTPGTPLTLT CTVSGFSLSS YAMSWVRQA |
| | | P GKGLEWIGII SSSGTTYYAS WAKGRFTISK TSTTVDL |
| | | KIT SPTTEDTATY FCARVDYYSS GWGGLWGPGT LVTVS |
| | | S |
| 16 | 77 -Lv full | AVEMTQTPAS MSAAVGGTVT IKCQASEDIY SNLAWYQQK |
| | | P GQPPKLLIYG ASTLASGVPS RFKGSGSGTE YTLTISG |
| | | VQC DDAATYYCQC TYDTSSYGNT FGGGTEMVVE |
| 17 | 110 CDR1 H | GFSLSSYAMI |
| 18 | 110 CDR2 H | IINSSDNTHY ATWAKG |
| 19 | 110 CDR3 H | DPYDYGYGWY FDL |
| 20 | 110 CDR1 L | QASQSVFNNK NLA |
| 21 | 110 CDR2 L | KASTLAS |
| 22 | 110 CDR3 L | QGEFSCSSTD CVT |
| 23 | 110-Hv full | QSVEESGGRL VTPGTPLTLT CTVSGFSLSS YAMIWVRQA |
| | | P GKGLEWIGII NSSDNTHYAT WAKGRFTISK TSTTVDL |
| | | KIT SPTTEDTATY FCARDPYDYG YGWYFDLWGP GTLVT |
| | | VSS |
| 24 | 110-Lv full | AQVLTQTPSP VSAAVGGTVT INCQASQSVF NNKNLAWYQ |
| | | Q KPGQPPKLLI YKASTLASGV SSRFKGSGSG TQFALTI |
| | | SGV QCDDAATYYC QGEFSCSSTD CVTFGGGTEV VVK |
| 25 | 111 CDR1 H | GFSLSTYAMS |
| 26 | 111 CDR2 H | IISSSGSTYY ASWAKG |
| 27 | 111 CDR3 H | ETWYWLDL |
| 28 | 111 CDR1 L | QASEDIYSNL A |
| 29 | 111 CDR2 L | GASTLAS |
| 30 | 111 CDR3 L | QCAYDSSSYG TP |
| 31 | 111-Hv full | QSVEESGGRL VTPGTPLTLT CTASGFSLST YAMSWVRQA |
| | | P GKGLEWIGII SSSGSTYYAS WAKGRFTISK TSTTVDL |
| | | KIT SPTTEDTATY FCARETWYWL DLWGQGTLVT VSS |
| 32 | 111-Lv full | AVEMTQTPAS VSAAVGGTVT INCQASEDIY SNLAWYQQK |
| | | P GQPPKLLIYG ASTLASGVPS RFKGSGSGTE YTLTISG |
| | | VQC DDAATYYCQC AYDSSSYGTP FGGGTEVVVK |
| 33 | 116-CDR1 H | GFSLSNYAMS |
| 34 | 116CDR2 H | IIYTTGFTYY ASWVKG |
| 35 | 116-CDR3 H | GLATYVSPPT RLDL |
| 36 | 116 CDR1 L | QASQSIYNSK NLA |
| 37 | 116 CDR2 L | SASTLAS |
| 38 | 116 CDR3 L | QGEFSCSSVD CAT |
| 39 | 116-Hv full | QSVEESGGRL VTPGTPLTLT CTVSGFSLSN YAMSWVRQA |
| | | P GKGLEWIGII YTTGFTYYAS WVKGRFTISK TSTTVDL |
| | | KIT SPTTEDTATY FCARGLATYV SPPTRLDLWG QGTLV |
| | | TVSS |
| 40 | 116-Lv full | AQVLTQTPSP VSAAVGGTVT INCQASQSIY |
| | | NSKNLAWYQQ KPGQPPKLLI YSASTLASGV |
| | | PSRFKGSGSG TQFTLTISDL ECDDAATYYC |
| | | QGEFSCSSVD CATFGGGTEV VVK |
| 41 | 16 H full | EVQLVESGGG LVKPGGSLKL SCAASGFTFS |
| | | NYAMSWVRQT PEKRLEWVAA INSNGGSAYY |
| | | PDTVKDRFTI SRDNAKNTLY LQMSSLRSED |
| | | TALYYCARRF YYDYGWFAYW GQGTLVTVSS |
| | | AKTTPPSVYP LAPGSAAQTN SMVTLGCLVK |
| | | GYFPEPVTVT WNSGSLSSGV HTFPAVLQSD |
| | | LYTLSSSVTV PSSTWPSETV TCNVAHPASS |
| | | TKVDKKIVPR DCGCKPCICT VPEVSSVFIF |
| | | PPKPKDVLTI TLTPKVTCVV VDISKDDPEV |
| | | QFSWFVDDVE VHTAQTQPRE EQFNSTFRSV |
| | | SELPIMHQDW LNGKEFKCRV NSAAFPAPIE |
| | | KTISKTKGRP KAPQVYTIPP PKEQMAKDKV |
| | | SLTCMITDFF PEDITVEWQW NGQPAENYKN |
| | | TQPIMDTDGS |
| | | YFVYSKLNVQ KSNWEAGNTF TCSVLHEGLH |
| | | NHHTEKSLSH SPGK |
| 42 | 16 L full | DIVLTQSPAS LSASVGETVT ITCRASENID |
| | | SYLAWYQQKQ GKSPOLLVYA ATLLADGVPS |
| | | RFSGSGSGTQ YSLKINSLOS EDVARYYCQH |
| | | YYSSPWTFGG GTKLEIKRTD AAPTVSIFPP |
| | | SSEQLTSGGA SVVCFLNNFY PKDINVKWKI |
| | | DGSERQNGVL NSWTDQDSKD STYSMSSTLT |
| | | LTKDEYERHN SYTCEATHKT STSPIVKSFN RNEC |
| 43 | 77a H full | QSVEESGGRL VTPGTPLTLT CTVSGFSLSS |
| | | YAMSWVRQAP GKGLEWIGII SSSGTTYYAS |
| | | WAKGRFTISK TSTTVDLKIT SPTTEDTATY |
| | | FCARVDYYSS GWGGLWGPGT LVTVSSAKTT |
| | | PPSVYPLAPG SAAQTNSMVT LGCLVKGYFP |
| | | EPVTVTWNSG SLSSGVHTFP AVLQSDLYTL |
| | | SSSVTVPSST WPSETVTCNV AHPASSTKVD |
| | | KKIVPRDCGC KPCICTVPEV SSVFIFPPKP |
| | | KDVLTITLTP KVTCVVVDIS KDDPEVQFSW |
| | | FVDDVEVHTA QTQPREEQEN STERSVSELP |
| | | IMHQDWLNGK EFKCRVNSAA FPAPIEKTIS |
| | | KTKGRPKAPQ VYTIPPPKEQ MAKDKVSLTC |
| | | MITDFFPEDI TVEWQWNGQP AENYKNTQPI |
| | | MDTDGSYFVY SKLNVQKSNW EAGNTFTCSV |
| | | LHEGLHNHHT EKSLSHSPGK |
| 44 | 77a L full | AVEMTQTPAS MSAAVGGTVT IKCQASEDIY |
| | | SNLAWYQQKP GQPPKLLIYG ASTLASGVPS |
| | | RFKGSGSGTE YTLTISGVQC DDAATYYCQC |
| | | TYDTSSYGNT FGGGTEMVVE RTDAAPTVSI |
| | | FPPSSEQLTS GGASVVCFLN NFYPKDINVK |
| | | WKIDGSERQN GVLNSWTDQD SKDCTYSMSS |
| | | TLTLTKDEYE RHNSYTCEAT HKTSTSPIVK SFNRNEC |
| 45 | 110 H full | QSVEESGGRL VTPGTPLTLT CTVSGFSLSS |
| | | YAMIWVRQAP GKGLEWIGII NSSDNTHYAT |
| | | WAKGRFTISK TSTTVDLKIT SPTTEDTATY |
| | | FCARDPYDYG YGWYFDLWGP GTLVTVSSAK |
| | | TTPPSVYPLA PGSAAQTNSM VTLGCLVKGY |
| | | FPEPVTVTWN SGSLSSGVHT FPAVLOSDLY |
| | | TLSSSVTVPS STWPSETVTC NVAHPASSTK |
| | | VDKKIVPRDC GCKPCICTVP EVSSVFIFPP |
| | | KPKDVLTITL TPKVTCVVVD ISKDDPEVQF |
| | | SWFVDDVEVH TAQTQPREEQ FNSTERSVSE |
| | | LPIMHQDWLN GKEFKCRVNS AAFPAPIEKT |
| | | ISKTKGRPKA PQVYTIPPPK EQMAKDKVSI |
| | | TCMITDFFPE DITVEWQWNG QPAENYKNTQ |
| | | PIMDTDGSYF VYSKLNVOKS NWEAGNTFTC |
| | | SVLHEGLHNH HTEKSLSHSP GK |
| 46 | 110 L full | AQVLTQTPSP VSAAVGGTVT INCQASQSVF |
| | | NNKNLAWYQQ KPGOPPKLLI YKASTLASGV |
| | | SSRFKGSGSG TQFALTISGV QCDDAATYYC |
| | | QGEFSCSSTD CVTFGGGTEV VVKRTDAAPT |
| | | VSIFPPSSEQ LTSGGASVVC FLNNFYPKDI |
| | | NVKWKIDGSE RQNGVLNSWT DODSKDCTYS |
| | | MSSTLTLTKD EYERHNSYTC EATHKTSTSP |
| | | IVKSFNRNEC |
| 47 | 111 H full | QSVEESGGRL VTPGTPLTLT CTASGFSLST |
| | | YAMSWVRQAP GKGLEWIGII SSSGSTYYAS |
| | | WAKGRFTISK TSTTVDLKIT SPTTEDTATY |
| | | FCARETWYWL DLWGQGTLVT VSSAKTTPPS |
| | | VYPLAPGSAA QTNSMVTLGC LVKGYFPEPV |
| | | TVTWNSGSLS SGVHTFPAVL QSDLYTLSSS |
| | | VTVPSSTWPS ETVTCNVAHP ASSTKVDKKI |
| | | VPRDCGCKPC ICTVPEVSSV FIFPPKPKDV |
| | | LTITLTPKVT CVVVDISKDD PEVQFSWFVD |
| | | DVEVHTAQTQ PREEQFNSTF RSVSELPIMH |
| | | QDWLNGKEFK CRVNSAAFPA PIEKTISKTK |
| | | GRPKAPQVYT IPPPKEQMAK DKVSLTCMIT |
| | | DFFPEDITVE WOWNGOPAEN YKNTQPIMDT |
| | | DGSYFVYSKL NVQKSNWEAG NTFTCSVLHE |
| | | GLHNHHTEKS LSHSPGK |
| 48 | 111 L full | AVEMTQTPAS VSAAVGGTVT INCQASEDIY |
| | | SNLAWYQQKP GOPPKLLIYG ASTLASGVPS |
| | | RFKGSGSGTE YTLTISGVQC DDAATYYCQC |
| | | AYDSSSYGTP FGGGTEVVVK RTDAAPTVSI |
| | | FPPSSEQLTS GGASVVCFLN NFYPKDINVK |
| | | WKIDGSERQN GVLNSWTDQD SKDCTYSMSS |
| | | TLTLTKDEYE RHNSYTCEAT HKTSTSPIVK SFNRNEC |
| 49 | 116 H full | QSVEESGGRL VTPGTPLTLT CTVSGFSLSN |
| | | YAMSWVRQAP GKGLEWIGII YTTGFTYYAS |
| | | WVKGRFTISK TSTTVDLKIT SPTTEDTATY |
| | | FCARGLATYV SPPTRLDLWG QGTLVTVSSA |
| | | KTTPPSVYPL APGSAAQTNS MVTLGCLVKG |
| | | YFPEPVTVTW NSGSLSSGVH TFPAVLQSDL |
| | | YTLSSSVTVP SSTWPSETVT CNVAHPASST |
| | | KVDKKIVPRD CGCKPCICTV PEVSSVFIFP |
| | | PKPKDVLTIT LTPKVTCVVV DISKDDPEVQ |
| | | FSWFVDDVEV HTAQTQPREE QFNSTERSVS |
| | | ELPIMHQDWL NGKEFKCRVN SAAFPAPIEK |
| | | TISKTKGRPK APQVYTIPPP KEQMAKDKVS |
| | | LTCMITDFFP EDITVEWQWN GQPAENYKNT |
| | | QPIMDTDGSY FVYSKLNVQK SNWEAGNTFT |
| | | CSVLHEGLHN HHTEKSLSHS PGK |
| 50 | 116 L full | AQVLTQTPSP VSAAVGGTVT INCQASQSIY |
| | | NSKNLAWYQQ KPGQPPKLLI YSASTLASGV |
| | | PSRFKGSGSG TQFTLTISDL ECDDAATYYC |
| | | QGEFSCSSVD CATFGGGTEV VVKRTDAAPT |
| | | VSIFPPSSEQ LTSGGASVVC FLNNFYPKDI |
| | | NVKWKIDGSE RQNGVLNSWT DODSKDCTYS |
| | | MSSTLTLTKD EYERHNSYTC EATHKTSTSP |
| | | IVKSFNRNEC |
| 51 | 16 H DNA | AAGCTTGCCA CCATGGAATG GAGCTGGGTC |
| | | TTTCTCTTCT TCCTGTCAGT AACTACAGGA |
| | | GTCCATTCTG AGGTGCAGCT GGTGGAGTCT |
| | | GGGGGAGGCT TAGTGAAGCC TGGAGGGTCC |
| | | CTGAAACTCT CCTGTGCAGC CTCTGGATTC |
| | | ACTTTCAGTA ACTATGCCAT GTCTTGGGTT |
| | | CGCCAGACTC CAGAGAAGAG GCTGGAGTGG |
| | | GTCGCAGCCA TTAATAGTAA TGGTGGTAGC |
| | | GCCTACTATC CAGACACTGT GAAGGACCGA |
| | | TTCACCATCT CCAGAGACAA TGCCAAGAAC |
| | | ACCCTGTACC TGCAAATGAG CAGTCTGAGG |
| | | TCTGAGGACA CAGCCTTGTA TTACTGTGCA |
| | | AGACGCTTCT ACTATGATTA CGGCTGGTTT |
| | | GCTTACTGGG GCCAAGGGAC TCTGGTCACA GTCTCGAGC |
| 52 | 16 L DNA | AAGCTTGCCA CCATGTCTGT CCCCACCCAA |
| | | GTCCTCGGAC TCCTGCTACT CTGGCTTACA |
| | | GATGCCAGAT GCGACATTGT GCTGACCCAA |
| | | TCTCCAGCTT CCCTGTCTGC ATCTGTGGGA |
| | | GAAACTGTCA CCATCACATG TCGAGCAAGT |
| | | GAGAATATTG ACAGTTATTT AGCATGGTAT |
| | | CAGCAGAAAC AGGGAAAATC TCCTCAGCTC |
| | | CTGGTCTATG CTGCAACACT CTTAGCAGAT |
| | | GGTGTGCCAT CAAGGTTCAG TGGCAGTGGA |
| | | TCAGGCACAC AGTATTCTCT CAAGATCAAC |
| | | AGCCTGCAGT CTGAAGATGT TGCGAGATAT |
| | | TACTGTCAAC ATTATTATAG TTCTCCGTGG |
| | | ACGTTCGGTG GAGGCACCAA GCTGGAAATA AAACGTACG |
| 53 | 77a H DNA | AAGCTTGCCA CCATGGAATG GAGCTGGGTC |
| | | TTTCTCTTCT TCCTGTCAGT AACTACAGGA |
| | | GTCCATTCTC AGTCGGTGGA GGAGTCCGGG |
| | | GGTCGCCTGG TCACGCCTGG GACACCCCTG |
| | | ACACTCACAT GCACAGTCTC TGGATTCTCC |
| | | CTCAGTAGCT ATGCGATGAG CTGGGTCCGC |
| | | CAGGCTCCAG GGAAGGGGCT GGAATGGATC |
| | | GGAATCATTA GTAGCAGTGG TACCACATAC |
| | | TACGCGAGCT GGGCGAAAGG CCGATTCACC |
| | | ATTTCCAAAA CCTCGACCAC GGTGGATCTG |
| | | AAAATCACCA GTCCGACAAC CGAGGACACG |
| | | GCCACCTATT TCTGTGCCAG AGTCGATTAC |
| | | TATAGTAGTG GCTGGGGTGG CTTGTGGGGC |
| | | CCAGGCACCC TGGTCACCGT CTCGAGC |
| 54 | 77a H DNA | AAGCTTCGAA GCCACCATGG ACACGAGGGC |
| | | CCCCACTCAG CTGCTGGGGC TCCTGCTGCT |
| | | CTGGCTCCCA GGTGCCACAT TTGCCGTTGA |
| | | AATGACCCAG ACTCCAGCCT CGATGTCTGC |
| | | CGCTGTGGGA GGCACAGTCA CCATCAAGTG |
| | | CCAGGCCAGT GAGGACATTT ATAGCAATTT |
| | | GGCCTGGTAT CAGCAGAAAC CAGGGCAGCC |
| | | TCCCAAGCTC CTGATCTATG GTGCATCCAC |
| | | TCTGGCTTCT GGGGTCCCAT CGCGGTTCAA |
| | | AGGCAGTGGA TCTGGGACAG AGTACACTCT |
| | | CACCATCAGC GGTGTGCAGT GTGACGATGC |
| | | TGCCACTTAC TATTGTCAAT GCACTTATGA |
| | | TACTAGTAGT TATGGTAATA CTTTCGGCGG |
| | | AGGGACCGAG ATGGTAGTCG AACGTACG |
| 55 | 110 H DNA | AAGCTTGCCA CCATGGAATG GAGCTGGGTC |
| | | TTTCTCTTCT TCCTGTCAGT AACTACAGGA |
| | | GTCCATTCTC AGTCGGTGGA GGAGTCCGGG |
| | | GGTCGCCTGG TCACGCCTGG GACACCCCTG |
| | | ACACTCACCT GCACAGTCTC TGGATTCTCC |
| | | CTCAGTAGCT ATGCAATGAT CTGGGTCCGC |
| | | CAGGCTCCAG GGAAGGGGCT GGAATGGATC |
| | | GGAATCATTA ATAGTAGTGA TAACACACAC |
| | | TACGCGACCT GGGCGAAAGG CCGATTCACC |
| | | ATCTCCAAAA CCTCGACCAC GGTGGATCTA |
| | | AAAATCACCA GTCCGACAAC CGAGGACACG |
| | | GCCACCTATT TCTGTGCCAG AGATCCCTAC |
| | | GACTATGGTT ATGGTTGGTA CTTTGACTTG |
| | | TGGGGCCCAG GCACCCTGGT CACCGTCTCG AGC |
| 56 | 110 L DNA | AAGCTTCGAA GCCACCATGG ACACGAGGGC |
| | | CCCCACTCAG CTGCTGGGGC TCCTGCTGCT |
| | | CTGGCTCCCA GGTGCCACAT TTGCCCAAGT |
| | | GCTGACCCAG ACTCCATCCC CTGTGTCTGC |
| | | AGCTGTGGGA GGCACAGTCA CCATCAACTG |
| | | CCAGGCCAGT CAGAGTGTTT TTAATAACAA |
| | | AAATTTAGCC TGGTATCAGC AGAAACCAGG |
| | | GCAGCCTCCC AAGCTCCTGA TCTACAAGGC |
| | | ATCCACTCTG GCATCTGGCG TCTCATCGCG |
| | | GTTCAAAGGC AGTGGATCTG GGACACAGTT |
| | | CGCTCTCACC ATCAGCGGCG TGCAGTGTGA |
| | | CGATGCTGCC ACTTACTACT GTCAAGGCGA |
| | | ATTTAGTTGT AGTAGTACTG ATTGCGTGAC |
| | | TTTCGGCGGA GGGACCGAGG TGGTGGTCAA ACGTACG |
| 57 | 111 H DNA | AAGCTTGCCA CCATGGAATG GAGCTGGGTC |
| | | TTTCTCTTCT TCCTGTCAGT AACTACAGGA |
| | | GTCCATTCTC AGTCGGTGGA GGAGTCCGGG |
| | | GGTCGCCTGG TCACGCCTGG GACACCCCTG |
| | | ACACTCACCT GCACAGCCTC TGGATTCTCC |
| | | CTCAGTACCT ATGCAATGAG TTGGGTCCGC |
| | | CAGGCTCCAG GGAAGGGGCT GGAATGGATC |
| | | GGAATCATTA GTAGTAGTGG TAGCACATAC |
| | | TACGCGAGCT GGGCGAAAGG CCGATTCACC |
| | | ATCTCCAAAA CCTCGACCAC GGTGGATCTG |
| | | AAAATCACCA GTCCGACAAC CGAGGACACG |
| | | GCCACCTATT TCTGTGCCAG AGAGACTTGG |
| | | TACTGGTTGG ATCTCTGGGG CCAGGGCACC |
| | | CTGGTCACCG TCTCGAGC |
| 58 | 111 L DNA | AAGCTTCGAA GCCACCATGG ACATGAGGGC |
| | | CCCCACTCAG CTGCTGGGGC TCCTGCTGCT |
| | | CTGGCTCCCA GGTGCCACAT TTGCCGTTGA |
| | | AATGACCCAG ACTCCAGCCT CGGTGTCTGC |
| | | CGCTGTGGGA GGCACAGTCA CCATCAATTG |
| | | CCAGGCCAGT GAGGACATTT ATAGCAATTT |
| | | GGCCTGGTAT CAGCAGAAAC CAGGGCAGCC |
| | | TCCCAAGCTC CTGATCTATG GTGCATCCAC |
| | | TCTGGCATCT GGGGTCCCAT CGCGGTTCAA |
| | | AGGCAGTGGA TCTGGGACAG AGTACACTCT |
| | | CACCATCAGC GGTGTGCAGT GTGACGATGC |
| | | TGCCACTTAC TACTGTCAAT GCGCTTATGA |
| | | TAGTAGTAGT TATGGTACCC CTTTCGGCGG |
| | | AGGGACCGAG GTGGTGGTCA AACGTACG |
| 59 | 116 H DNA | AAGCTTGCCA CCATGGAATG GAGCTGGGTC |
| | | TTTCTCTTCT TCCTGTCAGT AACTACAGGA |
| | | GTCCATTCTC AGTCGGTGGA GGAGTCCGGG |
| | | GGTCGCCTGG TCACGCCTGG GACACCCCTG |
| | | ACACTCACCT GCACAGTCTC TGGATTCTCC |
| | | CTCAGTAACT ATGCAATGAG CTGGGTCCGC |
| | | CAGGCTCCAG GGAAGGGGCT GGAATGGATC |
| | | GGAATCATTT ATACTACTGG TTTCACATAC |
| | | TACGCGAGCT GGGTGAAAGG CCGATTCACC |
| | | ATCTCCAAAA CCTCGACCAC GGTGGACCTG |
| | | AAAATCACCA GTCCGACAAC CGAGGACACG |
| | | GCCACCTATT TCTGTGCCAG AGGGCTGGCT |
| | | ACTTATGTTA GTCCCCCGAC TCGGTTGGAT |
| | | CTCTGGGGCC AGGGCACCCT GGTCACCGTC TCGAGC |
| 60 | 116 L DNA | AAGCTTCGAA GCCACCATGA ACATGAGGGC |
| | | CCCCACTCAG CTGCTGGGGC TCCTGCTGCT |
| | | CTGGCTCCCA GGTGCCACAT TTGCCCAAGT |
| | | GCTGACCCAG ACTCCATCCC CTGTGTCTGC |
| | | AGCTGTGGGA GGCACAGTCA CCATCAACTG |
| | | CCAGGCCAGT CAGAGTATTT ATAATAGCAA |
| | | AAATTTAGCC TGGTATCAGC AGAAACCAGG |
| | | GCAGCCTCCC AAGCTCCTGA TCTATTCTGC |
| | | ATCCACTCTG GCATCTGGGG TCCCATCGCG |
| | | GTTCAAAGGC AGTGGATCTG GGACACAGTT |
| | | CACTCTCACC ATCAGCGACC TGGAGTGTGA |
| | | CGATGCTGCC ACTTACTACT GTCAAGGCGA |
| | | ATTTAGTTGT AGTAGTGTTG ATTGCGCCAC |
| | | TTTCGGCGGA GGGACCGAGG TGGTGGTCAA ACGTACG |
| 61 | 16 CDR1 H | GGATTCACTT TCAGTAACTA TGCCATGTCT |
| 62 | 16 CDR2 H | GCCATTAATA GTAATGGTGG TAGCGCCTAC |
| | | TATCCAGACA CTGTGAAGGA C |
| 63 | 16 CDR3 H | CGCTTCTACT ATGATTACGG CTGGTTTGCT TAC |
| 64 | 16 CDR1 L | CGAGCAAGTG AGAATATTGA CAGTTATTTA GCA |
| 65 | 16 CDR2 L | GCTGCAACAC TCTTAGCAGA T |
| 66 | 16 CDR3 L | CAACATTATT ATAGTTCTCC GTGGACG |
| 67 | 77a CDR1 H | GGATTCTCCC TCAGTAGCTA TGCGATGAGC |
| 68 | 77a CDR2 H | ATCATTAGTA GCAGTGGTAC CACATACTAC |
| | | GCGAGCTGGG CGAAAGGC |
| 69 | 77a CDR3 H | GTCGATTACT ATAGTAGTGG CTGGGGTGGC TTG |
| 70 | 77a CDR1 L | CAGGCCAGTG AGGACATTTA TAGCAATTTG GCC |
| 71 | 77a CDR2 L | GGTGCATCCA CTCTGGCTTC T |
| 72 | 77a CDR3 L | CAATGCACTT ATGATACTAG TAGTTATGGT AATACT |
| 73 | 110 CDR1 H | GGATTCTCCC TCAGTAGCTA TGCAATGATC |
| 74 | 110 CDR2 H | ATCATTAATA GTAGTGATAA CACACACTAC |
| | | GCGACCTGGG CGAAAGGC |
| 75 | 110 CDR3 H | GATCCCTACG ACTATGGTTA TGGTTGGTAC TTTGACTTG |
| 76 | 110 CDR1 L | CAGGCCAGTC AGAGTGTTTT TAATAACAAA AATTTAGCC |
| 77 | 110 CDR2 L | AAGGCATCCA CTCTGGCATC T |
| 78 | 110 CDR3 L | CAAGGCGAAT TTAGTTGTAG TAGTACTGAT TGCGTGACT |
| 79 | 111 CDR1 H | GGATTCTCCC TCAGTACCTA TGCAATGAGT |
| 80 | 111 CDR2 H | ATCATTAGTA GTAGTGGTAG CACATACTAC |
| | | GCGAGCTGGG CGAAAGGC |
| 81 | 111 CDR3 H | GAGACTTGGT ACTGGTTGGA TCTC |
| 82 | 111 CDRI L | CAGGCCAGTG AGGACATTTA TAGCAATTTG GCC |
| 83 | 111 CDR2 L | GGTGCATCCA CTCTGGCATC T |
| 84 | 111 CDR3 L | CAATGCGCTT ATGATAGTAG TAGTTATGGT ACCCCT |
| 85 | 116 CDR1 H | GGATTCTCCC TCAGTAACTA TGCAATGAGC |
| 86 | 116 CDR2 H | ATCATTTATA CTACTGGTTT CACATACTAC |
| | | GCGAGCTGGG TGAAAGGC |
| 87 | 116 CDR3 H | GGGCTGGCTA CTTATGTTAG TCCCCCGACT |
| | | CGGTTGGATC TC |
| 88 | 116 CDR1 L | CAGGCCAGTC AGAGTATTTA TAATAGCAAA AATTTAGCC |
| 89 | 116 CDR2 L | TCTGCATCCA CTCTGGCATC T |
| 90 | 116 CDR3 L | CAAGGCGAAT TTAGTTGTAG TAGTGTTGAT TGCGCCACT |

Underlined portions of any DNA sequence above denote a signal sequence.

1. (canceled)

2. An antibody or antigen binding fragment thereof which binds to CD1a, comprising:
a) a heavy chain variable region comprising a CDR3 of SEQ ID NO: 35 or a sequence having at least 80% identity thereto, and/or a light chain variable region comprising a CDR3 of SEQ ID NO: 38 or a sequence having at least 80% identity thereto; or
b) a heavy chain variable region comprising a complementarity determining region CDR3 of SEQ ID NO: 3 or a sequence having at least 80% thereto, and/or a light chain variable region comprising a CDR3 of SEQ ID NO: 6 or a sequence having at least 80% identity thereto; or
c) a heavy chain variable region comprising a CDR3 of SEQ ID NO: 11 or a sequence having at least 80% identity thereto, and/or a light chain variable region comprising a CDR3 of SEQ ID NO: 14 or a sequence having at least 80% identity thereto; or
d) a heavy chain variable region comprising a CDR3 of SEQ ID NO: 19 or a sequence having at least 80% identity thereto, and/or a light chain variable region comprising a CDR3 of SEQ ID NO: 22 or a sequence having at least 80% identity thereto; or
e) a heavy chain variable region comprising a CDR3 of SEQ ID NO: 27 or a sequence having at least 80% identity thereto, and/or a light chain variable region comprising a CDR3 of SEQ ID NO: 30 or a sequence having at least 80% identity thereto.

3. An antibody or antigen binding fragment thereof which binds to CD1a, comprising:
a) a heavy chain variable region comprising:
a CDR1 of SEQ ID NO: 33,
a CDR2 of SEQ ID NO: 34, and
a CDR3 of SEQ ID NO: 35,
or sequences having at least 80% identity thereto, and/or
a light chain variable region comprising:
a CDR1 of SEQ ID NO: 36,
a CDR2 of SEQ ID NO: 37, and
a CDR3 of SEQ ID NO: 38,
or sequences having at least 80%, identity thereto; or
b) a heavy chain variable region comprising:
a CDR1 of SEQ ID NO: 1,
a CDR2 of SEQ ID NO: 2, and
a CDR3 of SEQ ID NO: 3,
or sequences having at least 80% identity thereto, and/or
a light chain variable region comprising:
a CDR1 of SEQ ID NO: 4,
a CDR2 of SEQ ID NO: 5, and
a CDR3 of SEQ ID NO: 6,
or sequences having at least 80%, identity thereto; or
c) a heavy chain variable region comprising:
a CDR1 of SEQ ID NO: 9,
a CDR2 of SEQ ID NO: 10, and
a CDR3 of SEQ ID NO: 11,
or sequences having at least 80% identity thereto, and/or
a light chain variable region comprising:
a CDR1 of SEQ ID NO: 12,
a CDR2 of SEQ ID NO: 13, and
a CDR3 of SEQ ID NO: 14,
or sequences having at least 80%, identity thereto; or
d) a heavy chain variable region comprising:
a CDR1 of SEQ ID NO: 17,
a CDR2 of SEQ ID NO: 18, and
a CDR3 of SEQ ID NO: 19,
or sequences having at least 80% identity thereto, and/or
a light chain variable region comprising:
a CDR1 of SEQ ID NO: 20,
a CDR2 of SEQ ID NO: 21, and
a CDR3 of SEQ ID NO: 22,
or sequences having at least 80%, identity thereto; or
e) a heavy chain variable region comprising:
a CDR1 of SEQ ID NO: 25,
a CDR2 of SEQ ID NO: 26, and
a CDR3 of SEQ ID NO: 27,
or sequences having at least 80% identity thereto, and/or
a light chain variable region comprising:
a CDR1 of SEQ ID NO: 28,
a CDR2 of SEQ ID NO: 29, and
a CDR3 of SEQ ID NO: 30,
or sequences having at least 80%, identity thereto.

4. An antibody or antigen binding fragment thereof which binds to CD1a, comprising:
a) a heavy chain variable region comprising or consisting of SEQ ID NO: 39; and/or a light chain variable region comprising or consisting of SEQ ID NO: 40
or sequences having at least 80% identity thereto; or
b) a heavy chain variable region comprising or consisting of SEQ ID NO: 7; and/or a light chain variable region comprising or consisting of SEQ ID NO: 8
or sequences having at least 80% identity thereto; or
c) a heavy chain variable region comprising or consisting of SEQ ID NO: 15; and/or a light chain variable region comprising or consisting of SEQ ID NO: 16
or sequences having at least 80% identity thereto; or
d) a heavy chain variable region comprising or consisting of SEQ ID NO: 23; and/or a light chain variable region comprising or consisting of SEQ ID NO: 24
or sequences having at least 80% identity thereto; or
e) a heavy chain variable region comprising or consisting of SEQ ID NO: 31; and/or a light chain variable region comprising or consisting of SEQ ID NO: 32
or sequences having at least 80% identity thereto.

5. An antibody or antigen binding fragment thereof which binds to CD1a, comprising:
a) a heavy chain comprising or consisting of SEQ ID NO: 49; and/or a light chain comprising or consisting of SEQ ID NO: 50
or sequences having at least 80% identity thereto; or
b) a heavy chain comprising or consisting of SEQ ID NO: 41; and/or a light chain comprising or consisting of SEQ ID NO: 42
or sequences having at least 80% identity thereto; or
c) a heavy chain comprising or consisting of SEQ ID NO: 43; and/or a light chain comprising or consisting of SEQ ID NO: 44
or sequences having at least 80% identity thereto; or
d) a heavy chain comprising or consisting of SEQ ID NO: 45; and/or a light chain comprising or consisting of SEQ ID NO: 46
or sequences having at least 80% identity thereto; or
e) a heavy chain comprising or consisting of SEQ ID NO: 47; and/or a light chain comprising or consisting of SEQ ID NO: 48
or sequences having at least 80% identity thereto.

6. The antibody or antigen binding fragment thereof of claim 2, wherein the antibody or antigen binding fragment thereof includes an ScFv or other modified format.

7. The antibody or antigen binding fragment thereof of claim 2, wherein the antibody or antigen binding fragment thereof is modified to stabilise and/or extend the half-life; optionally wherein the modification is PEGylation.

8. The antibody or antigen binding fragment thereof of claim 2, wherein the antibody or antigen binding fragment thereof is humanized.

9. The antibody or antigen binding fragment thereof of claim 2, wherein the antibody or antigen binding fragment thereof is a human IgG1 isotype or a human IgG4 isotype or other natural or modified isotype.

10. The antibody or antigen binding fragment thereof of claim 2, wherein the antibody or antigen binding fragment thereof is bispecific or multispecific.

11. A nucleic acid encoding the antibody or antigen binding fragment thereof of claim 2.

12. A vector comprising the nucleic acid of claim 11, optionally wherein the vector is an expression vector, a plasmid, or a viral vector.

13. (canceled)

14. A host cell comprising the antibody or antigen binding fragment thereof of claim 2, optionally wherein the host cell is a bacterial cell or a mammalian cell.

15. (canceled)

16. A pharmaceutical composition comprising one or more antibody or antigen binding fragment thereof of claim 2.

17-25. (canceled)

26. A method of treating one or more inflammatory skin or mucosal disease or disorder, or one or more associated systemic disease or disorder, or one or more inflammatory drug reaction which manifests systemically, or one or more CD1a-expressing malignancy, in a subject in need thereof, comprising administering to the subject an effective amount of one or more antibody or antigen binding fragment thereof of claim 2.

27. The method of claim 26, wherein the one or more antibody or antigen binding fragment thereof comprise or consist of two antibodies or antigen binding fragments thereof which bind to CD1a, each comprising or consisting of:
a) a first antibody or antigen binding fragment thereof having a heavy chain variable region comprising:
a CDR1 of SEQ ID NO: 33, a CDR2 of SEQ ID NO: 34, and a CDR3 of SEQ ID NO: 35, or sequences having at least 80% identity thereto, and
a light chain variable region comprising:
a CDR1 of SEQ ID NO: 36, a CDR2 of SEQ ID NO: 37, and a CDR3 of SEQ ID NO: 38, or sequences having at least 80%, identity thereto; and
a second antibody or antigen binding fragment thereof having a heavy chain variable region comprising:
a CDR1 of SEQ ID NO: 1, a CDR2 of SEQ ID NO: 2, and a CDR3 of SEQ ID NO: 3, or sequences having at least 80% identity thereto, and
a light chain variable region comprising:
a CDR1 of SEQ ID NO: 4, a CDR2 of SEQ ID NO: 5, and a CDR3 of SEQ ID NO: 6, or sequences having at least 80%, identity thereto; or
b) a first antibody or antigen binding fragment thereof having a heavy chain variable region comprising or consisting of SEQ ID NO: 39; and a light chain variable region comprising or consisting of SEQ ID NO: 40, or sequences having at least 80% identity thereto; and
a second antibody or antigen binding fragment thereof having a heavy chain variable region comprising or consisting of SEQ ID NO: 7; and a light chain variable region comprising or consisting of SEQ ID NO: 8, or sequences having at least 80% identity thereto; or
c) a first antibody or antigen binding fragment thereof having a heavy chain comprising or consisting of SEQ ID NO: 49; and a light chain comprising or consisting of SEQ ID NO: 50, or sequences having at least 80% identity thereto; and
a second antibody or antigen binding fragment thereof having a heavy chain comprising or consisting of SEQ ID NO: 41; and a light chain comprising or consisting of SEQ ID NO: 42, or sequences having at least 80% identity thereto.

28. A method of monitoring treatment efficacy or disease status in a subject diagnosed with a CD1a-expressing malignancy, comprising:
i. providing a biological sample obtained from the subject;
ii. determining the level of binding of one or more antibodies or antigen binding fragments of claim 2 to CD1a-expressing cells in the sample obtained from the subject before treatment, or at intervals between treatments, or at time intervals in the absence of treatment;
iii. determining that the treatment is effective, or that the disease status is improving, if the tumour volume, or level of binding of the one or more antibodies or antigen binding fragment thereof of claim 2 to CD1a-expressing cells, is reduced after treatment or between treatment intervals or at time intervals in the absence of treatment, optionally wherein the reduction in tumour volume or level of binding of the one or more antibodies or antigen binding fragment thereof to CD1a-expressing cells is by 25% or more.

29. The method of claim 26, wherein
a) the one or more inflammatory skin or mucosal disease or disorder is one or more of:
i) a predominantly neutrophilic skin disease such as acne, generalized pustular psoriasis, plaque psoriasis, guttate psoriasis, palmoplantar pustulosis, SAPHO syndrome, acute febrile neutrophilic dermatosis (Sweet syndrome), histiocytoid neutrophilic dermatitis, neutrophilic dermatosis of the dorsal hands, pyoderma gangrenosum, neutrophilic eccrine hidradenitis, hidradenitis suppurativa, erythema elevatum diutinum, Behcet disease, bowel-associated dermatitis-arthritis syndrome, other infection-associated inflammation, neutrophilic urticarial dermatosis, palisading neutrophilic granulomatous dermatitis, erythema gyratum repens, neutrophilic annular erythema, acute generalised exanthematous pustulosis (AGEP), vasculitis and others;
ii) an autoimmune disorder such as connective tissue disease (eg lupus, dermatomyositis, scleroderma/systemic sclerosis, Churg Strauss syndrome), panniculitis, vasculitides, autoimmune blistering conditions (eg bullous pemphigoid, pemphigus, linear IgA disease), dermatitis herpetiformis, coeliac disease, some auto-inflammatory disease, vitiligo, alopecia areata, alopecia universalis, alopecia totalis, panniculitis, lichen planus, erythema multiforme, lichen sclerosis, other lichenoid and erythema multiforme-like diseases, psoriatic arthritis, inflammatory bowel disease, rheumatoid arthritis, multiple sclerosis, Guillain-Barre syndrome, transverse myelitis, thyroiditis, neurodegeneration and others;
iii) mast cell disorders and eosinophilic disorders, such as Muckle Wells syndrome, eosinophilia and systemic symptoms syndrome, urticaria, angioedema, keratoconjunctivitis, food allergy, other allergy or atopy including atopic dermatitis, rhinitis, conjunctivitis, asthma, eosinophilic oesophagitis and other eosinophilic mucosal diseases, contact dermatitis and others;
iv) Graft vs host disease; and
v) Other drug reactions which manifest as an inflammatory skin or mucosal disease or disorder including Stevens Johnsons syndrome, toxic epidermal necrolysis, drug reaction with eosinophilia and systemic symptoms syndrome (DRESS) and acute generalised exanthematous pustulosis (AGEP), erythema multiforme, bullous, fixed drug, and other drug reactions which manifest as an inflammatory skin or mucosal disease or disorder; or
(b) the one or more associated systemic disease or disorder, or one or more inflammatory drug reaction which manifests systemically, is an inflammatory reaction to Aldara (imiquimod); or
(c) the CD1a-expressing malignancy is one or more of Langerhans cell histiocytosis, a T cell lymphoma or a thymoma.

30. The method of claim 26, wherein the one or more inflammatory skin or mucosal disease or disorder is one or more of psoriasis, dermatitis, lupus erythematosus, or drug reactions which manifest as an inflammatory skin or mucosal disease or disorder.

31. The method of claim 26, wherein the one or more antibody or antigen binding fragment thereof is administered in combination with one or more other therapeutic agent, optionally wherein the one or more other therapeutic agent is selected from the group consisting of cytotoxic agents, anti-inflammatory agents such as steroids, CAR-T cells such as regulatory or cytolytic CAR-T cells, and other cells expressing or presenting one or more antibody or antigen binding fragment which bind CD1a.
